(19) United States
(12) Patent Application Publication
(10) Pub. No.: US 2024/0352510 A1
(43) Pub. Date: Oct. 24, 2024

(54) CAPTURING AND AMPLIFYING POLYNUCLEOTIDES

(71) Applicant: Illumina, Inc., San Diego, CA (US)

(72) Inventors: Mathieu Lessard-Viger, San Diego, CA (US); Rebecca Turk-MacLeod, San Diego, CA (US); Vanessa Montaño-Machado, San Diego, CA (US); Jeffrey Fisher, San Diego, CA (US); Rohit Subramanian, San Diego, CA (US); Krishnarjun Sarkar, San Diego, CA (US); Sahngki Hong, San Diego, CA (US); Weixian Xi, San Diego, CA (US); Brandon Wenning, San Diego, CA (US); Lewis Kraft, Acworth, GA (US); Wayne George, Haverhill (GB); Brian Mather, San Diego, CA (US); Allison Meade, San Diego, CA (US); John Daly, San Diego, CA (US)

(73) Assignee: Illumina, Inc., San Diego, CA (US)

(21) Appl. No.: 18/633,966

(22) Filed: Apr. 12, 2024

Related U.S. Application Data
(60) Provisional application No. 63/496,351, filed on Apr. 14, 2023.

(51) Int. Cl. C12Q 1/6848; C12Q 1/6855

(52) U.S. Cl. CPC C12Q 1/6848; C12Q 1/6855

(57) ABSTRACT

In some examples, a structure is contacted with polynucleotides having a variety of lengths and each including first and second adapters. The structure includes a substrate including first and second regions spaced apart from one another by a gap of at least 100 nm, a first set of capture primers coupled to the first region of the substrate, and a second set of capture primers coupled to the second region of the substrate. The first adapter of the polynucleotide is hybridized to a capture primer of the first set of capture primers. Based on that polynucleotide being sufficiently long to bridge the gap, it is amplified using the first and second sets of capture primers. Based upon that polynucleotide being insufficiently long to bridge the gap, it is not amplified. Optionally, a wall may be disposed in the gap.

### CROSS-REFERENCE TO RELATED APPLICATIONS

This application claims the benefit of U.S. Provisional Patent Application No. 63/496,351, filed on Apr. 14, 2023 and entitled “Capturing and Amplifying Polynucleotides,” the entire contents of which are incorporated by reference herein.

### INCORPORATION BY REFERENCE OF SEQUENCE LISTING

The material in the accompanying sequence listing is hereby incorporated by reference into the application. The accompanying sequence listing XML file, named “IP-2504-US.xml”, was created on Apr. 11, 2024 and is 20 KB in size.

### FIELD

This application generally relates to capturing and amplifying polynucleotides.

### BACKGROUND

Cluster amplification is an approach to amplifying polynucleotides, for example for use in genetic sequencing. Target polynucleotides are captured by primers (e.g., P5 and P7 primers) coupled to a substrate surface in a flowcell, and form “seeds” at random locations on the surface. Cycles of amplification are performed to form clusters on the surface around each seed. The clusters include copies, and complementary copies, of the seed polynucleotides. In some circumstances, the substrate is patterned so as to define regions that bound different clusters, such as wells that may be filled with respective clusters.

Different library preparation methods for fragmenting or tagmenting DNA or RNA for sequencing may generate a variety of lengths of polynucleotides, ranging from relatively short (e.g., less than about 150 base pairs (bp)) to relatively long (e.g., about 500 bp or greater). Different commercially known sequencers are known to amplify relatively short polynucleotides significantly more efficiently than relatively long polynucleotides; for further details, see Gohl et al., “Measuring sequencer size bias using Recount: a novel method for highly accurate Illumina sequencing-based quantification,” Genome Biology 20: article number 85 (2019), the entire contents of which are incorporated by reference herein. Such a bias towards amplification of relatively short polynucleotides may, in some cases, reduce the value of certain information obtained from sequencing those polynucleotides.

### SUMMARY

Examples provided herein are related to capturing and amplifying polynucleotides. Structures, compositions, and methods for performing such capture and amplification are disclosed.

Some examples herein provide a method of amplifying a polynucleotide. The method may include contacting a structure with a fluid comprising polynucleotides having a variety of lengths. Each of the polynucleotides may include first and second adapters. The structure may include a substrate including a first region and a second region spaced apart from one another by a gap of at least 100 nm; a first set of capture primers coupled to the first region of the substrate; and a second set of capture primers coupled to the second region of the substrate. The method may include hybridizing the first adapter of a polynucleotide from the fluid to a capture primer of the first set of capture primers. The method may include, based upon that polynucleotide being sufficiently long to bridge the gap, amplifying that polynucleotide using the first set of capture primers and the second set of capture primers. The method may include, based upon that polynucleotide being insufficiently long to bridge the gap, not amplifying that polynucleotide.

In some examples, the gap has length A, and based upon the polynucleotide being about A/0.34 nm bases long or greater, the polynucleotide is amplified, and based upon the polynucleotide being less than about A/0.34 nm bases long, the polynucleotide is not amplified.

In some examples, the structure further includes a wall disposed within the gap between the first region and the second region. In some examples, the first region is located within a first recess of the substrate, the second region is located within a second recess of the substrate, and the wall divides the first recess from the second recess.

In some examples, the structure further includes a vertical sidewall including the first region.

In some examples, the structure includes a raised surface including the first region.

In some examples, the first region surrounds the second region.

In some examples, the second region surrounds the first region.

In some examples, the first region and the second region are at least partially coplanar with one another.

In some examples, the first region and the second region are at least partially vertically separated from one another.

In some examples, the second set of capture primers includes a mixture of first and second types of capture primers. In some examples, the first set of capture primers consists essentially of a third type of capture primer. In some examples, the third type of capture primer includes a seeding primer.

In some examples, the first set of capture primers includes amplification primers of a first type. In some examples, the second set of capture primers includes amplification primers of a second type that is different from the first type.

In some examples, the first set of capture primers includes seeding primers of a first type. In some examples, the second set of capture primers includes seeding primers of a second type that is different from the first type of the first seeding primers. In some examples, the first adapter includes a first seeding adapter which is shorter than the seeding primers of the first type, and the first polynucleotide includes a second seeding adapter which is shorter than the seeding primers of the second type. In some examples, the first seeding adapter is single-stranded, and the second seeding adapter is single-stranded. In some examples, based upon that polynucleotide being sufficiently long to bridge the gap: the first seeding adapter and one of the seeding primers of the first type of the seeding primers form a first duplex, and the second seeding adapter and one of the seeding primers of the second type of the seeding primers form a second duplex. The first and second duplex together may sufficiently hold the polynucleotide to the structure to amplify the polynucleotide. In some examples, based upon that polynucleotide being insufficiently long to bridge the gap: the first seeding adapter and one of the seeding primers of the first type of the seeding primers form a first duplex, or the second seeding adapter and one of the seeding primers of the second type of the seeding primers form a second duplex. The first duplex alone may insufficiently hold the polynucleotide to the structure to amplify the polynucleotide, and the second duplex alone may insufficiently hold the polynucleotide to the structure to amplify the polynucleotide. In some examples, the first duplex has a melting temperature of about 35-50° C.; and the second duplex has a melting temperature of about 35-50° C. In some examples, the structure further includes amplification primers.

In some examples, the first adapter is single-stranded.

In some examples, the polynucleotide further includes a second adapter, which is double-stranded. In some examples, a capture primer of the second set of capture primers binds to the second adapter using strand invasion.

Some examples herein provide a structure. The structure may include a substrate including a first region and a second region spaced apart from one another by a gap of at least 100 nm. The structure may include a first set of capture primers coupled to the first region of the substrate. The structure may include a second set of capture primers coupled to the second region of the substrate. The structure may include a polynucleotide including a first adapter hybridized to a capture primer of the first set of capture primers, and a second adapter hybridized to a capture primer of the second set of capture primers.

Some examples herein provide a structure. The structure may include a material having a first recess and a second recess defined therein, the first recess being separated from the second recess by a wall. The structure may include a first hydrogel disposed within the first recess. The structure may include a second hydrogel disposed within the second recess.

In some examples, the wall separates the first hydrogel from the second hydrogel.

In some examples, the first hydrogel is coupled to a first set of capture primers, and the second hydrogel is coupled to a second set of capture primers. In some examples, capture primers of the first set are of a different type than capture primers of the second set.

In some examples, the first set of capture primers includes seeding primers. In some examples, the second set of capture primers includes amplification primers.

In some examples, the first set of capture primers includes a first set of amplification primers. In some examples, the second set of capture primers includes a second set of amplification primers.

In some examples, the first hydrogel is disposed on sidewalls of the first region of the recess, the sidewalls being formed at least by the first layer and the wall.

In some examples, the second hydrogel is disposed on sidewalls within the first portion of the second region of the recess, the sidewalls being formed at least by the first layer and the wall.

In some examples, the first hydrogel is on or in a first particle disposed within the first recess. In some examples, the first and second recesses are different sizes. In some examples, the second hydrogel is on or in a second particle disposed within the second recess. In some examples, the first and second particles are different sizes than one another.

Some examples herein provide a method. The method may include disposing a first particle within a first recess of a substrate. The substrate may include a second recess and a wall separating the first recess from the second recess. A first hydrogel may be disposed within the second recess of the substrate.

In some examples, the first particle includes a second hydrogel. In some examples, the method further includes disposing the first hydrogel within the second recess of the substrate. In some examples, disposing the first hydrogel within the second recess includes, while the first particle is disposed within the first recess, disposing a second particle within the second recess. In some examples, the first particle is larger than the second particle. In some examples, the first particle is larger than the second recess and thus is size excluded from becoming disposed within the second recess when disposing the first particle within the first recess. In some examples, the first particle being disposed within the first recess inhibits the second particle from becoming disposed within the first recess.

In some examples, the first recess is larger than the second recess.

In some examples, the first particle is coupled to a first set of capture primers, and the first hydrogel is coupled to a second set of capture primers. In some examples, capture primers of the first set are of a different type than capture primers of the second set.

In some examples, the first set of capture primers includes seeding primers. In some examples, the second set of capture primers includes amplification primers.

In some examples, the first set of capture primers includes a first set of amplification primers. In some examples, the second set of capture primers includes a second set of amplification primers. In some examples, the first particle is coupled to the first set of capture primers after the first particle is disposed within the first recess. In some examples, the first hydrogel is coupled to the second set of capture primers after the second hydrogel is disposed within the second recess.

Some examples herein provide a structure. The structure may include a substrate having first and second recesses therein, the first recess being separated from the second recess by a wall. The structure may include a first particle within the first recess. The structure may include a second particle within the second recess.

Some examples herein provide a method of capturing a double-stranded polynucleotide. The method may include contacting a structure with a double-stranded polynucleotide including first and second double-stranded amplification adapters and first and second single-stranded seeding adapters. The structure may include a substrate, first and second amplification primers coupled to the substrate, and first and second seeding primers coupled to the substrate. The method may include hybridizing the first single-stranded seeding adapter to the first seeding primer to form a first duplex. The method may include, while the first single-stranded seeding adapter is hybridized to the first seeding primer, hybridizing the second single-stranded seeding adapter to the second seeding primer to form a second duplex. The first and second duplexes together may retain the double-stranded polynucleotide at the substrate for sufficient time to amplify the double-stranded polynucleotide using the first and second double-stranded amplification adapters and the first and second amplification primers. The first duplex alone, or the second duplex alone, may not retain the double-stranded polynucleotide at the substrate for sufficient time to amplify the double-stranded polynucleotide.

In some examples, the first and second seeding primers are shorter than the first and second amplification primers.

In some examples, the first and second duplexes have a reduced number of G-C pairs than does a duplex between the first amplification adapter and the first amplification primer, and have a reduced number of G-C pairs than does a duplex between the second amplification adapter and the second amplification primer. In some examples, the first and second duplexes have a melting temperature of about 35-50° C. In some examples, a duplex between the first amplification adapter and the first amplification primer, and a duplex between the second amplification adapter and the second amplification primer, have a melting temperature of about 55-65° C.

In some examples, the first seeding primer is coupled to a different region of the substrate than is the second seeding primer.

In some examples, the first seeding primer is coupled to a different region of the substrate than is the first amplification primer.

In some examples, the first seeding primer is coupled to a different region of the substrate than is the second amplification primer.

In some examples, the first seeding primer is located in a first region of the substrate in which substantially only first seeding primers are located, and the second seeding primer is located in a second region of the substrate in which substantially only second seeding primers are located. In some examples, the first and second amplification primers are located in a third region of the substrate in which a mixture of first and second amplification primers is located.

In some examples, the first and second regions are coplanar with one another. In some examples, the third region is coplanar with the first and second regions.

In some examples, the first and second regions are in different planes than one another. In some examples, the third region is in a different plane than at least one of the first and second regions. In some examples, the third region is in a different plane than both of the first and second regions.

In some examples, the first and second seeding primers and the first and second amplification primers are located in a region of the substrate in which a mixture of first and second seeding primers and first and second amplification primers is located.

In some examples, the first and second seeding adapters have the same sequence as one another.

In some examples, the first and second seeding primers have the same sequence as one another, and a different sequence than the first and second amplification primers.

In some examples, the first and second seeding adapters have different sequences than one another, and different sequences than the first and second amplification primers. In some examples, the first seeding primers and a first mixture of the first and second amplification primers are located in a first region of the substrate, and the second seeding primers and a second mixture of the first and second amplification primers are located in a second region of the substrate. In some examples, the first amplification primers in the first region of the substrate include a first excision moiety, and the second amplification primers in the second region of the substrate include a second excision moiety that is orthogonal to the first excision moiety. In some examples, the first and second duplexes retain the double-stranded polynucleotide at approximately an interface between the first and second regions of the substrate.

Some examples herein provide a composition. The composition may include a structure including a substrate, first and second amplification primers coupled to the substrate, and first and second seeding primers coupled to the substrate. The composition may include a double-stranded polynucleotide including first and second double-stranded amplification adapters and first and second single-stranded seeding adapters. The first single-stranded seeding adapter may be hybridized to the first seeding primer to form a first duplex. The second single-stranded seeding adapter may be hybridized to the second seeding primer to form a second duplex. The first and second duplexes together may retain the double-stranded polynucleotide at the substrate for sufficient time to amplify the double-stranded polynucleotide using the first and second double-stranded amplification adapters and the first and second amplification primers. The first duplex alone, or the second duplex alone, may not retain the double-stranded polynucleotide at the substrate for sufficient time to amplify the double-stranded polynucleotide.

In some examples, the first and second seeding primers are shorter than the first and second amplification primers.

In some examples, the first and second duplexes have a reduced number of G-C pairs than does a duplex between the first amplification adapter and the first amplification primer, and have a reduced number of G-C pairs than does a duplex between the second amplification adapter and the second amplification primer.

In some examples, the first and second duplexes have a melting temperature of about 35-50° C.

In some examples, a duplex between the first amplification adapter and the first amplification primer, and a duplex between the second amplification adapter and the second amplification primer, have a melting temperature of about 55-65° C.

In some examples, the first seeding primer is coupled to a different region of the substrate than is the second seeding primer.

In some examples, the first seeding primer is coupled to a different region of the substrate than is the first amplification primer.

In some examples, the first seeding primer is coupled to a different region of the substrate than is the second amplification primer.

In some examples, the first seeding primer is located in a first region of the substrate in which substantially only first seeding primers are located, and the second seeding primer is located in a second region of the substrate in which substantially only second seeding primers are located. In some examples, the first and second amplification primers are located in a third region of the substrate in which a mixture of first and second amplification primers is located.

In some examples, the first and second regions are coplanar with one another. In some examples, the third region is coplanar with the first and second regions.

In some examples, the first and second regions are in different planes than one another. In some examples, the third region is in a different plane than at least one of the first and second regions. In some examples, the third region is in a different plane than both of the first and second regions.

In some examples, the first and second seeding primers and the first and second amplification primers are located in a region of the substrate in which a mixture of first and second seeding primers and first and second amplification primers is located.

In some examples, the first and second seeding adapters have the same sequence as one another.

In some examples, the first and second seeding primers have the same sequence as one another, and a different sequence than the first and second amplification primers.

In some examples, the first and second seeding primers have different sequences than one another, and different sequences than the first and second amplification primers.

In some examples, the first seeding primers and a first mixture of the first and second amplification primers are located in a first region of the substrate, and the second seeding primers and a second mixture of the first and second amplification primers are located in a second region of the substrate. In some examples, the first amplification primers in the first region of the substrate include a first excision moiety, and the second amplification primers in the second region of the substrate include a second excision moiety that is orthogonal to the first excision moiety.

In some examples, the first and second duplexes retain the double-stranded polynucleotide at approximately an interface between the first and second regions of the substrate.

Some examples herein provide a method of capturing a double-stranded polynucleotide. The method may include contacting a structure with a scaffold. The structure may include a substrate, first and second amplification primers coupled to the substrate, and first and second seeding primers coupled to the substrate. The scaffold may include first and second single-stranded seeding adapters and a third seeding primer. The method may include hybridizing the first single-stranded seeding adapter to the first seeding primer to form a first duplex. The method may include, while the first single-stranded seeding adapter is hybridized to the first seeding primer, hybridizing the second single-stranded seeding adapter to the second seeding primer to form a second duplex. The method may include contacting the scaffold with a double-stranded polynucleotide including first and second double-stranded amplification adapters and a third single-stranded seeding adapter. The method may include hybridizing the third single-stranded seeding adapter to the third seeding primer to form a third duplex. The first and second duplexes together may retain the scaffold at the substrate for sufficient time to amplify the double-stranded polynucleotide using the first and second double-stranded amplification adapters and the first and second amplification primers. The first duplex alone, or the second duplex alone, may not retain the scaffold at the substrate for sufficient time to amplify the double-stranded polynucleotide.

In some examples, the third single-stranded seeding adapter is hybridized to the third seeding primer while the first single-stranded seeding adapter is hybridized to the first seeding primer and while the second single-stranded seeding adapter is hybridized to the second seeding primer. In other examples, the third single-stranded seeding adapter is hybridized to the third seeding primer before the first single-stranded seeding adapter is hybridized to the first seeding primer and before the second single-stranded seeding adapter is hybridized to the second seeding primer.

In some examples, the scaffold includes at least one of a nanoparticle, a DNA dendrimer, a polymer dendrimer, a peptide dendrimer, a polymer with bottlebrush structure, a single strand DNA with bottlebrush structure, a single strand DNA produced by rolling circle amplification, or a polypeptide scaffold.

In some examples, the first and second seeding primers are shorter than the first and second amplification primers.

In some examples, the first and second duplexes have a reduced number of G-C pairs than does a duplex between the first amplification adapter and the first amplification primer, and have a reduced number of G-C pairs than does a duplex between the second amplification adapter and the second amplification primer.

In some examples, the first and second duplexes have a melting temperature of about 35-50° C.

In some examples, the third duplex has a melting temperature of about 55-65° C.

In some examples, a duplex between the first amplification adapter and the first amplification primer, and a duplex between the second amplification adapter and the second amplification primer, have a melting temperature of about 55-65° C.

In some examples, the first and second seeding primers and the first and second amplification primers are located in a region of the substrate in which a mixture of first and second seeding primers and first and second amplification primers is located.

In some examples, the first and second seeding adapters have the same sequence as one another.

In some examples, the first and second seeding primers have the same sequence as one another, and a different sequence than the first and second amplification primers.

In some examples, the third seeding primer has a different sequence than the first and second seeding primers.

In some examples, the first and second amplification adapters have different sequences than one another, and the first and second amplification primers have different sequences than one another.

In some examples, the first and second seeding adapters have different sequences than one another.

In some examples, the first and second seeding primers and the first and second amplification primers have different sequences than one another. In some examples, the first seeding primers and a first mixture of the first and second amplification primers are located in a first region of the substrate, and the second seeding primers and a second mixture of the first and second amplification primers are located in a second region of the substrate. In some examples, the first amplification primers in the first region of the substrate include a first excision moiety, and the second amplification primers in the second region of the substrate include a second excision moiety that is orthogonal to the first excision moiety. In some examples, the first and second duplexes retain the scaffold at approximately an interface between the first and second regions of the substrate.

Some examples herein provide a composition. The composition may include a structure including a substrate, first and second amplification primers coupled to the substrate, and first and second seeding primers coupled to the substrate. The composition may include a scaffold including first and second single-stranded seeding adapters and a third seeding primer. The first single-stranded seeding adapter may be hybridized to the first seeding primer to form a first duplex. The second single-stranded seeding adapter may be hybridized to the second seeding primer to form a second duplex. The composition may include double-stranded polynucleotide including first and second double-stranded amplification adapters and a third single-stranded seeding adapter. The third single-stranded seeding adapter may be hybridized to the third seeding primer to form a third duplex. The first and second duplexes together may retain the scaffold at the substrate for sufficient time to amplify the double-stranded polynucleotide using the first and second double-stranded amplification adapters and the first and second amplification primers. The first duplex alone, or the second duplex alone, may not retain the scaffold at the substrate for sufficient time to amplify the double-stranded polynucleotide.

In some examples, the scaffold includes at least one of a nanoparticle, a DNA dendrimer, a polymer dendrimer, a peptide dendrimer, a polymer with bottlebrush structure, a single strand DNA with bottlebrush structure, a single strand DNA produced by rolling circle amplification, or a polypeptide scaffold.

In some examples, the first and second seeding primers are shorter than the first and second amplification primers.

In some examples, the first and second duplexes have a reduced number of G-C pairs than does a duplex between the first amplification adapter and the first amplification primer, and have a reduced number of G-C pairs than does a duplex between the second amplification adapter and the second amplification primer.

In some examples, the first and second duplexes have a melting temperature of about 35-50° C.

In some examples, the third duplex has a melting temperature of about 55-65° C.

In some examples, a duplex between the first amplification adapter and the first amplification primer, and a duplex between the second amplification adapter and the second amplification primer, have a melting temperature of about 55-65° C.

In some examples, the first and second seeding primers and the first and second amplification primers are located in a region of the substrate in which a mixture of first and second seeding primers and first and second amplification primers is located.

In some examples, the first and second seeding adapters have the same sequence as one another.

In some examples, the first and second seeding primers have the same sequence as one another, and a different sequence than the first and second amplification primers.

In some examples, the third seeding primer has a different sequence than the first and second seeding primers.

In some examples, the first and second amplification adapters have different sequences than one another, and the first and second amplification primers have different sequences than one another.

In some examples, the first and second seeding adapters have different sequences than one another.

In some examples, the first and second seeding primers and the first and second amplification primers have different sequences than one another. In some examples, the first seeding primers and a first mixture of the first and second amplification primers are located in a first region of the substrate, and the second seeding primers and a second mixture of the first and second amplification primers are located in a second region of the substrate. In some examples, the first amplification primers in the first region of the substrate include a first excision moiety, and the second amplification primers in the second region of the substrate include a second excision moiety that is orthogonal to the first excision moiety. In some examples, the first and second duplexes retain the scaffold at approximately an interface between the first and second regions of the substrate.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1I schematically illustrate example structures and operations for capturing and amplifying polynucleotides using first and second sets of capture primers that are spaced apart from one another by a gap.

FIGS. 2, 3, 4, 5A-5B, 6, 7, and 8A-8B schematically illustrate additional example structures and operations for capturing and amplifying polynucleotides in a manner such as described with reference to FIGS. 1A-1I.

FIGS. 9A-9F schematically illustrate additional example structures and operations for capturing and amplifying polynucleotides using first and second sets of capture primers that are spaced apart from one another by a gap.

FIGS. 10A-10E schematically illustrate example structures and operations for capturing and amplifying polynucleotides using seeding adapters and first and second sets of seeding primers that are spaced apart from one another by a gap.

FIGS. 11, 12A-12B, 13, 14, 15, 16 schematically illustrate additional example structures and operations for capturing and amplifying polynucleotides in a manner such as described with reference to FIGS. 1A-11, 9A-9F, 10A-10E, 22A-22C, 23A-23F, 25A-25G, and 26A-26E.

FIGS. 17, 18, and 19A-19B schematically illustrate example structures and operations for disposing different hydrogels in respective recesses in a substrate.

FIGS. 20A-20G schematically illustrate example operations for forming recesses in a substrate.

FIGS. 21A-21C schematically illustrate additional example structures and operations for disposing different hydrogels in respective recesses in a substrate.

FIGS. 22A-22C schematically illustrate additional example structures and operations for capturing and amplifying polynucleotides using seeding adapters and seeding primers.

FIGS. 23A-23F schematically illustrate additional example structures and operations for capturing and amplifying polynucleotides using seeding adapters and seeding primers.

FIGS. 24A-24C schematically illustrate alternative duplexes that may be formed between seeding adapters and seeding primers in examples such as described with reference to FIGS. 10A-10E, 12A-12B, 22A-22C, and 23A-23F.

FIGS. 25A-25G schematically illustrate additional example structures and operations for capturing and amplifying polynucleotides using seeding adapters and seeding primers.

FIGS. 26A-26E schematically illustrate additional example structures and operations for capturing and amplifying polynucleotides using seeding adapters and seeding primers.

FIG. 27A illustrates a non-limiting example of a polynucleotide bottlebrush structure that may be used in examples such as described with reference to FIGS. 23A-23F.

FIG. 27B illustrates a non-limiting example of a polynucleotide bottlebrush structure that may be used in examples such as described with reference to FIGS. 26A-26E.

FIG. 28 illustrates fluorescence intensity from clusters respectively formed using double-stranded nucleotides or using scaffolds, for different lengths of seeding primers.

### DETAILED DESCRIPTION

Examples provided herein are related to capturing and amplifying polynucleotides. Structures and methods for performing such capture and amplification are disclosed.

Some examples herein relate to selectively capturing and amplifying relatively long polynucleotides. Such examples may, for example, reduce or eliminate the bias toward relatively short polynucleotides such as described in Gohl, while remaining compatible both with standard library preparation techniques that generate a variety of polynucleotide lengths, and with standard reagents, equipment, and processes for performing sequencing-by-synthesis (SBS). In a manner such as described in greater detail herein, relatively long polynucleotides may be selectively amplified using different types of capture primers that are spatially separated from one another. Each such type of capture primer may be needed for polynucleotide seeding and/or amplification. Because the different types of capture primers are spatially separated from one another, polynucleotides that are sufficiently long to reach both types of capture primers are amplified, while polynucleotides that cannot reach both types of capture primers may not be amplified. A variety of different structures for spatially separating capture primers from one another, and methods of making such structures are provided herein. Additionally, or alternatively, some examples herein may be used to capture and amplify polynucleotides with improved monoclonality.

First, some terms used herein will be briefly explained. Then, some example structures and example methods for capturing and amplifying polynucleotides will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to +10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes have polynucleotide strands that disassociate from one another. Polynucleotides that are “partially” hybridized to one another means that they have sequences that are complementary to one another, but such sequences are hybridized with one another along only a portion of their lengths to form a partial duplex. Polynucleotides with an “inability” to hybridize include those which are physically separated from one another such that an insufficient number of their bases may contact one another in a manner so as to hybridize with one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosinc diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, isocytosine, isoguaninc, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosinc, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” is defined as a polynucleotide to which nucleotides may be added via a free 3′ OH group. A primer may include a 3′ block preventing polymerization until the block is removed. A primer may include a modification at the 5′ terminus to allow a coupling reaction or to couple the primer to another moiety. A primer may include one or more moieties which may be cleaved under suitable conditions, such as UV light, chemistry, enzyme, or the like. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer.

A “capture primer” is intended to mean a primer that is coupled to the substrate and may hybridize to an adapter of the target polynucleotide. In some cases, a capture primer that is coupled to the substrate and may hybridize to another adapter of that target polynucleotide may be referred to as an “orthogonal capture primer.” The adapters may have respective sequences that are complementary to those of capture primers to which they may hybridize. A capture primer and an orthogonal capture primer may have different and independent sequences than one another. A capture primer that may be used to hybridize to an adapter of a target polynucleotide in order to couple that polynucleotide to the substrate, but that may not be used to grow a complementary strand during an amplification process, may in some cases be referred to as a “seeding primer.” A capture primer that may be used to grow a complementary strand during an amplification process may in some cases be referred to as an “amplification primer.”

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kchagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible. In some examples, a substrate is a bead or a flow cell.

In some examples, a substrate includes a patterned surface. A “patterned surface” refers to an arrangement of different regions in or on an exposed layer of a substrate. For example, one or more of the regions may be features where one or more capture primers are present. The features can be separated by interstitial regions where capture primers are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of features (e.g., microwells or nanowells) on glass, silicon, plastic or other suitable material(s) with a patterned, covalently-linked hydrogel such as poly(N-(5-azidoacetamidylpentyl) acrylamide-co-acrylamide) (PAZAM). The process creates hydrogel regions used for sequencing that may be stable over sequencing runs with a large number of cycles. The covalent linking of the hydrogel to the wells may be helpful for maintaining the hydrogel in the structured features throughout the lifetime of the structured substrate during a variety of uses. However in many examples, the hydrogel need not be fully or even partially covalently linked to the wells. For example, in some conditions silane free acrylamide (SFA) may be used as the hydrogel material.

In particular examples, a structured substrate may be made by patterning a suitable material with wells (e.g. microwells or nanowells), coating the patterned material with a hydrogel material (e.g., PAZAM, SFA or chemically modified variant thereof, such as the azidolyzed version of SFA (azido-SFA)) and polishing the surface of the hydrogel coated material, for example via chemical or mechanical polishing, thereby retaining hydrogel in the wells but removing or inactivating substantially all of the hydrogel from the interstitial regions on the surface of the structured substrate between the wells. Primers may be attached to hydrogel material. A solution including a plurality of target polynucleotides (e.g., a fragmented human genome or portion thereof) may then be contacted with the polished substrate such that individual target polynucleotides will seed individual wells via interactions with primers attached to the hydrogel material; however, the target polynucleotides will not occupy the interstitial regions due to absence or inactivity of the hydrogel material. Amplification of the target polynucleotides may be confined to the wells because absence or inactivity of hydrogel in the interstitial regions may inhibit outward migration of the growing cluster. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An exemplary patterned substrate having these characteristics is the etched substrate used in connection with BEAD ARRAY technology (Illumina, Inc., San Diego, Calif.).

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that may be used in methods and compositions set forth herein include, but are not limited to, those commercially available from Illumina, Inc. (San Diego, CA).

As used herein, a “hydrogel” refers to a three-dimensional polymer network structure that includes polymer chains and is at least partially hydrophilic and contains water within spaces between the polymer chains. A hydrogel may include any suitable combination of hydrophilic, hydrophobic, and/or amphiphilic polymer(s), so long as the overall polymer network is hydrophilic and contains water within spaces between the polymer chains. Hydrogels include chemical hydrogels in which both the bonding to form the polymer chains, and any cross-linking between the polymer chains, is covalent; such cross-linking during hydrogel formation may be irreversible, as distinguished from the present reversible cross-linking which is performed after the hydrogel is formed. In some cases, the chemical hydrogel may include, or may consist essentially of, brush-like structures of polymer chains attached to a surface, substantially without physical or covalent crosslinks between polymer chains, or alternatively polymer chains with multiple attachment points to a surface, resulting in loops, but also lacking interchain crosslinks. Hydrogels also include physical hydrogels in which the bonding to form the polymer chains, and any cross-linking within the polymer chains, is not covalent. Nonlimiting examples of physical hydrogels include agarose and alginate.

As used herein, the “polymer chain” of a hydrogel is intended to mean those portions of the hydrogel that are polymerized with one another during the polymerization process. Polymer chains may be cross-linked to form the hydrogel. For example, cross-linkers may be added during or after the polymerization process that forms the polymer chains. Additionally, or alternatively, in some examples the polymer chains may be deposited on a substrate surface that includes functional groups to which functional groups of the polymer chains become coupled. The polymer chains may be coupled to the surface, e.g., via reactions between the functional groups of the polymer chains and the functional groups at the surface, and such coupling may cross-link the polymer chains to form the hydrogel. Such cross-linking may cause the polymer chains to covalently or non-covalently attach to one another, or may occur as a result of chain entanglement during polymerization and/or attachment to a surface.

As used herein, the term “directly” when used in reference to a layer covering the surface of a substrate is intended to mean that the layer covers the substrate's surface without a significant intermediate layer, such as, e.g., an adhesive layer or a polymer layer. Layers directly covering a surface may be attached to this surface through any chemical or physical interaction, including covalent bonds or non-covalent adhesion.

As used herein, the term “immobilized” when used in reference to a polynucleotide is intended to mean direct or indirect attachment to a substrate via covalent or non-covalent bond(s). In certain examples, covalent attachment may be used, or any other suitable attachment in which the polynucleotides remain stationary or attached to a substrate under conditions in which it is intended to use the substrate, for example, in polynucleotide amplification or sequencing. Polynucleotides to be used as capture primers or as target polynucleotides may be immobilized such that a 3′-end is available for enzymatic extension and at least a portion of the sequence is capable of hybridizing to a complementary sequence. Immobilization may occur via hybridization to a surface attached oligonucleotide, in which case the immobilized oligonucleotide or polynucleotide may be in the 3′-5′ orientation. Alternatively, immobilization may occur by means other than base-pairing hybridization, such as covalent attachment.

As used herein, the term “array” refers to a population of substrate regions that may be differentiated from each other according to relative location. Different molecules (such as polynucleotides) that are at different regions of an array may be differentiated from each other according to the locations of the regions in the array. An individual region of an array may include one or more molecules of a particular type. For example, a substrate region may include a single target polynucleotide having a particular sequence, or a substrate region may include several polynucleotides having the same sequence (or complementary sequences thereof). The regions of an array respectively may include different features than one another on the same substrate. Exemplary features include without limitation, wells in a substrate, beads (or other particles) in or on a substrate, projections from a substrate, ridges on a substrate or channels in a substrate. The regions of an array respectively may include different regions on different substrates than each other. Different molecules attached to separate substrates may be identified according to the locations of the substrates on a surface to which the substrates are associated or according to the locations of the substrates in a liquid or hydrogel. Exemplary arrays in which separate substrates are located on a surface include, without limitation, those having beads in wells.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above exemplary ranges. Exemplary polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded,” when used in reference to a polynucleotide, is intended to mean that all or substantially all of the nucleotides in the polynucleotide are hydrogen bonded to respective nucleotides in a complementary polynucleotide. A “partially” double stranded polynucleotide may have at least about 10%, at least about 25%, at least about 50%, at least about 60%, at least about 70%, at least about 80%, at least about 90% or at least about 95% of its nucleotides, but fewer than all of its nucleotides, hydrogen bonded to nucleotides in a complementary polynucleotide.

As used herein, the term “single-stranded,” when used in reference to a polynucleotide, means that essentially none of the nucleotides in the polynucleotide are hydrogen bonded to a respective nucleotide in a complementary polynucleotide. A polynucleotide that has an “inability” to hybridize to another polynucleotide may be single-stranded.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed. A target polynucleotide hybridized to a capture primer may include nucleotides that extend beyond the 5′ or 3′ end of the capture oligonucleotide in such a way that not all of the target polynucleotide is amenable to extension. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “amplicon,” when used in reference to a polynucleotide, is intended to means a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

A substrate region that includes substantially only amplicons of a given polynucleotide may be referred to as “monoclonal,” while a substrate region that includes amplicons of polynucleotides having different sequences than one another may be referred to as “polyclonal.” A substrate region that includes a sufficient number of amplicons of a given polynucleotide to be used to sequence that polynucleotide maybe referred to as “functionally monoclonal.” Illustratively a substrate region in which about 60% or greater of the amplicons are of a given polynucleotide may be considered to be “functionally monoclonal.” Additionally, or alternatively, a substrate region from which about 60% or more of a signal is from amplicons of a given polynucleotide may be considered to be “functionally monoclonal.” A polyclonal region of a substrate may include different subregions therein that respectively are monoclonal. Each such monoclonal region, whether within a larger polyclonal region or on its own, may correspond to a “cluster” generated from a “seed.” The “seed” may refer to a single target polynucleotide, while the “cluster” may refer to a collection of amplicons of that target

### Methods for Capturing and Amplifying Polynucleotides

Some examples provided herein relate to generating clusters that are biased towards relatively long target polynucleotides and that are substantially monoclonal, by providing different types of capture primers in different regions of the substrate such that polynucleotides which are sufficiently long to reach each type of capture primer may be amplified, and polynucleotides which are not sufficiently long to reach each type of capture primer substantially may not be amplified. Some examples herein are particularly well suited to generating clusters for use in simultaneous paired-end reads in which an amplified polynucleotide's sequence is read using SBS in a first substrate region, and that polynucleotide's complementary sequence is read using SBS in a second substrate region, in parallel with one another, but it should be understood that the examples are generally applicable to any type of cluster.

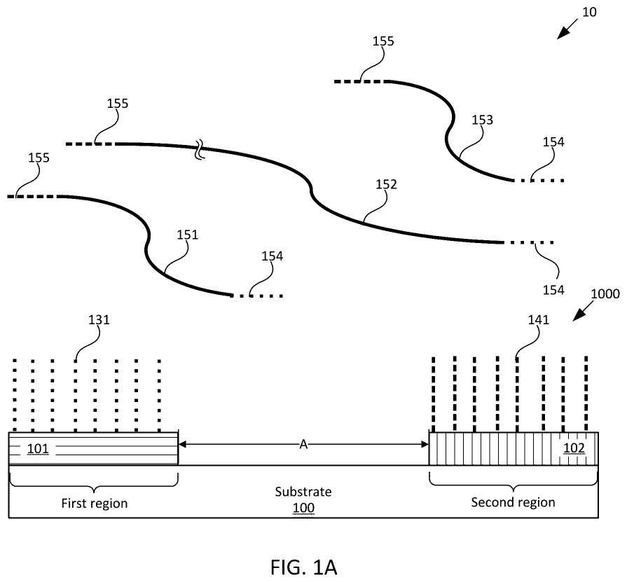

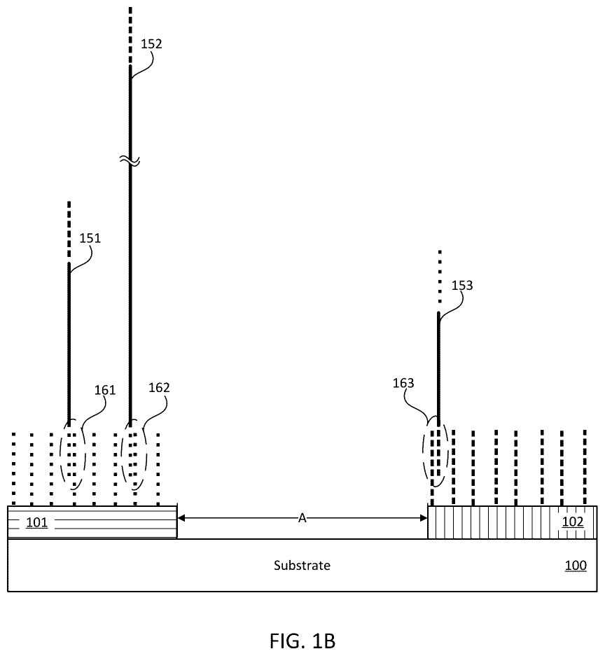

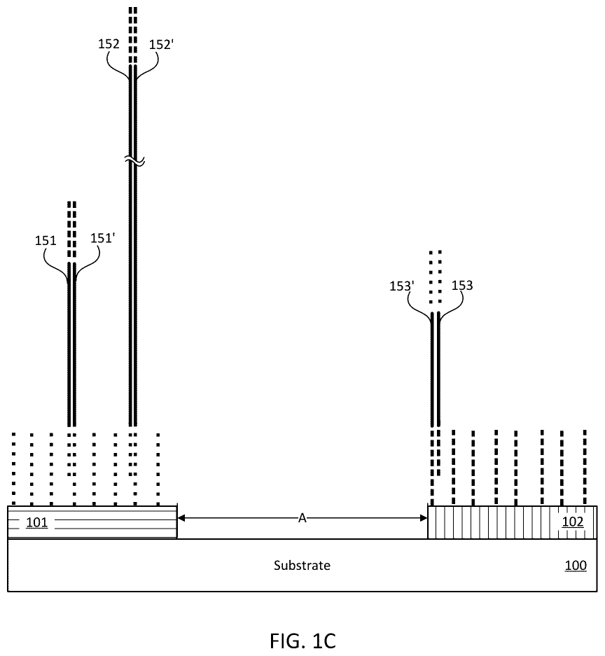

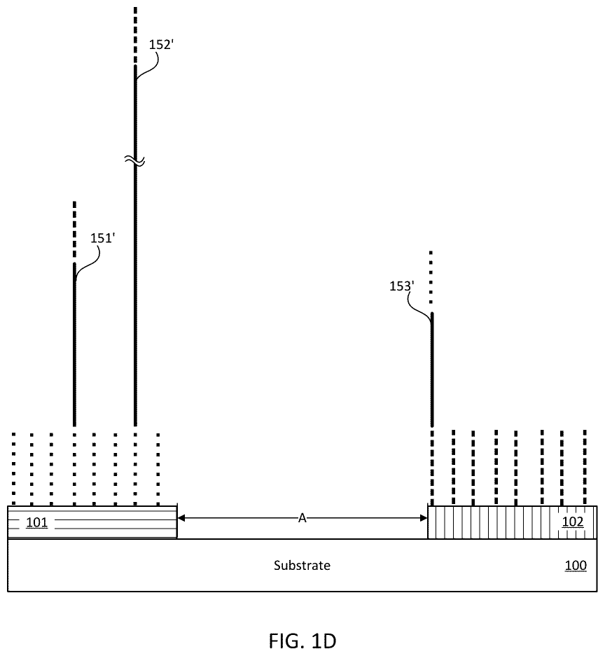

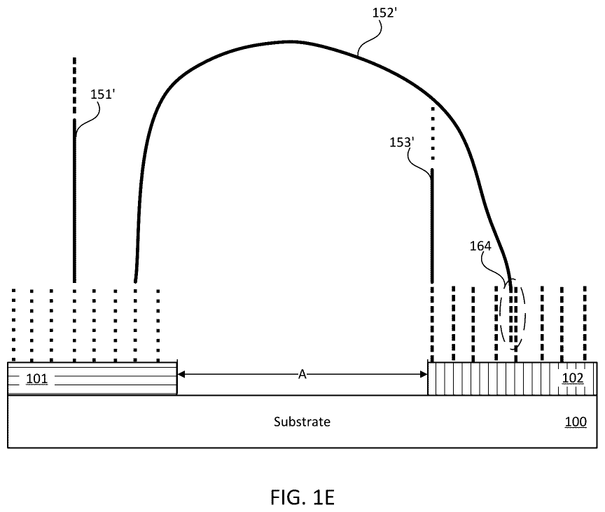

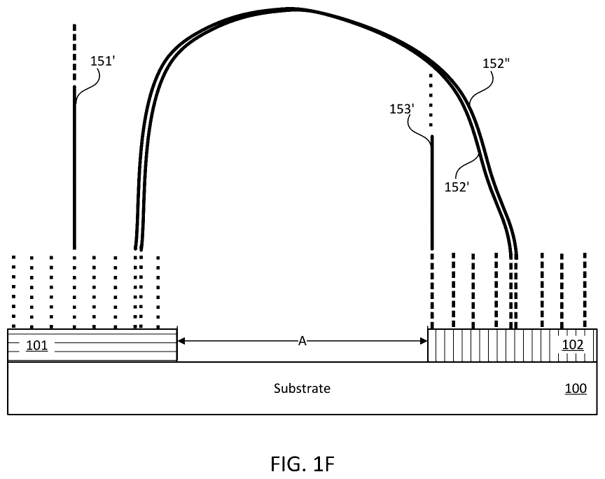

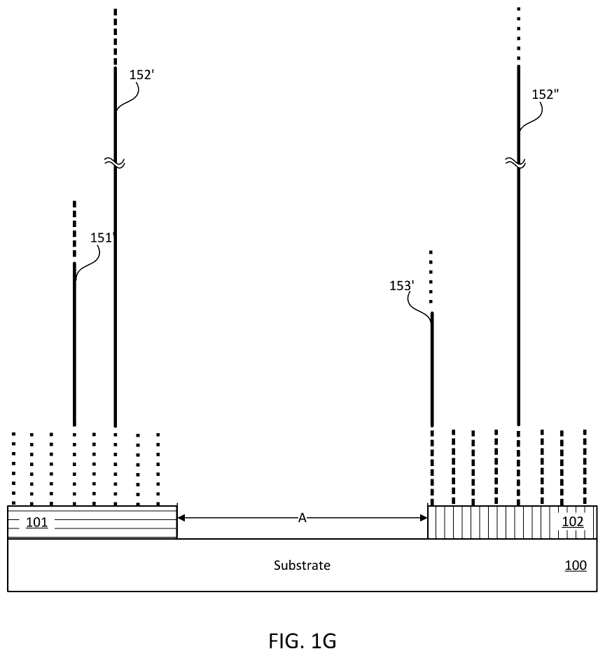

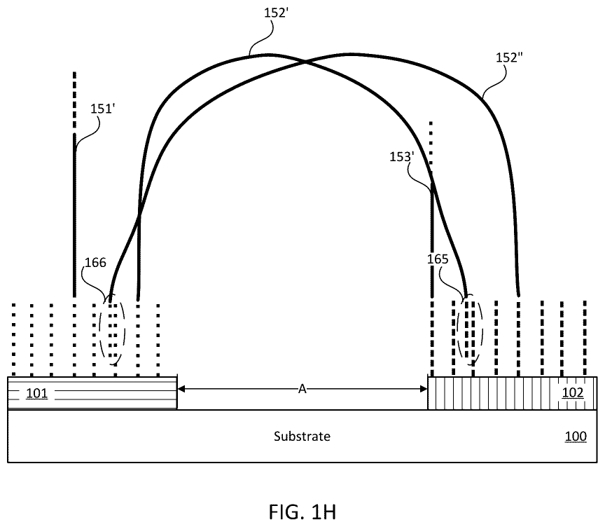

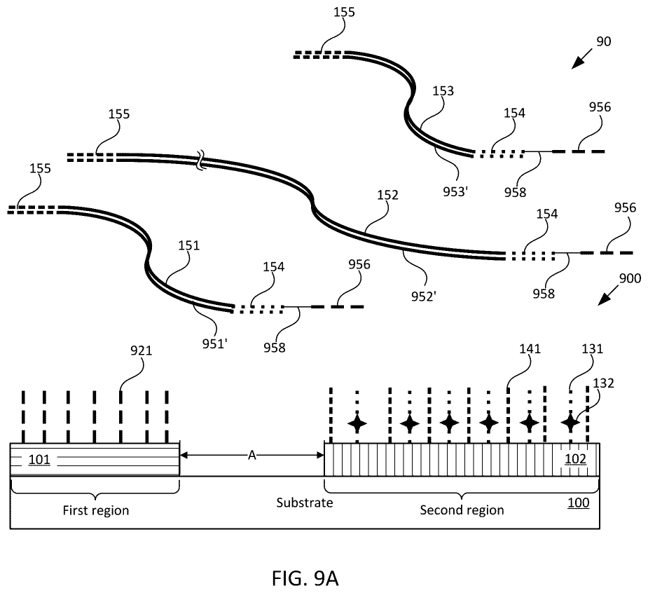

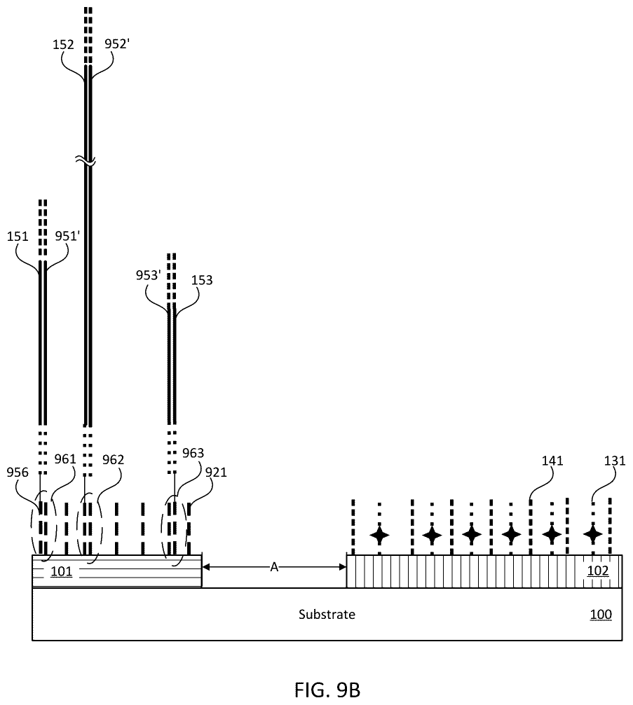

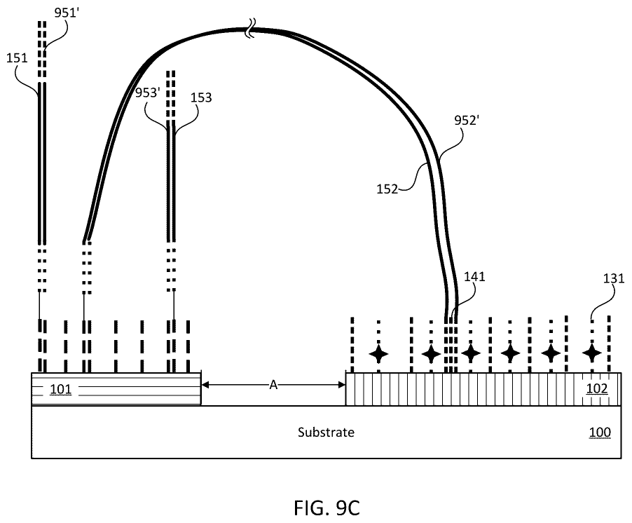

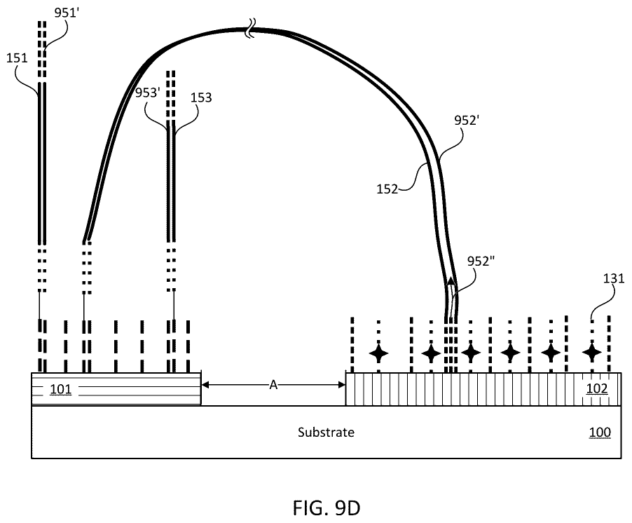

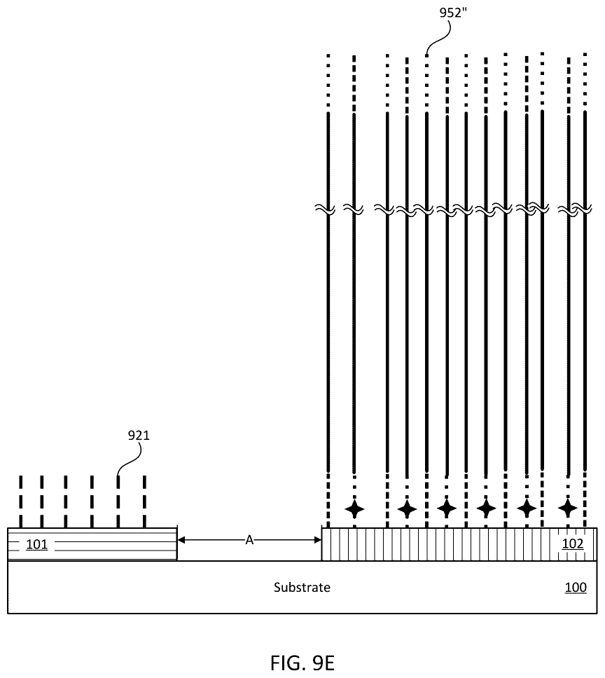

For example, FIGS. 1A-1I schematically illustrate example structures and operations for capturing and amplifying target polynucleotides using first and second sets of capture primers that are spaced apart from one another by a gap. Referring first to FIG. 1A, structure 1000 may be contacted with fluid 10. Fluid 10 may include target polynucleotides having a variety of lengths, e.g., polynucleotide fragments that are generated using commercially available fragmentation or tagmentation techniques, using DNA or RNA that it is desired to sequence. Illustratively, fluid 10 may include polynucleotide 151 which is of intermediate length, polynucleotide 152 which is relatively long, and polynucleotide 153 which is relatively short. Although only three polynucleotides are illustrated in FIG. 1A, it will be appreciated that fluid 10 may include thousands, or even millions, of fragments of different lengths than one another. Each of the polynucleotides 151, 152, 153 may include first and second adapters, e.g., first adapter 154 and second adapter 155. In a manner such as will be described further below with reference to FIGS. 9A-9F, 10A-10E, 11, 12A-12B, 22A-22C, 23A-23F, 24A-24C, 25A-25G, and 26A-26E, polynucleotides 151, 152, 153 optionally may further include one or more additional adapters, e.g., seeding adapters. While the polynucleotides (and all of their adapters) are illustrated as being single-stranded in FIGS. 1A-1I, in other examples herein the polynucleotides may be double-stranded, and their adapters may be single stranded or double-stranded, or a combination thereof, depending on the example.

In the nonlimiting example illustrated in FIG. 1A, structure 1000 may include substrate 100 comprising a first region and a second region spaced apart from one another by a gap (A) of at least 100 nm. A set of capture primers 131 may be coupled to the first region of the substrate 100; and a set of capture primers 141 may be coupled to the second region of the substrate. Illustratively, the set of capture primers 131 may be coupled to a first hydrogel 101 that is disposed on the first region of the substrate 100, and the set of capture primers 141 may be coupled to a second hydrogel that is disposed on the second region of the substrate 100. Capture primers 131 and 141 may have any suitable sequences, e.g., that are orthogonal to one another. That is, capture primer 141 may be referred to as an orthogonal capture primer, while capture primer 131 may be referred to as a capture primer; or capture primer 131 may be referred to as an orthogonal capture primer, while capture primer 141 may be referred to as a capture primer. In one nonlimiting example, capture primers 131 may include P5, and capture primers 141 may include P7. In another nonlimiting example, capture primers 141 may include P5, and capture primers 131 may include P7. Although FIG. 1A illustrates an example in which the first set of capture primers consists essentially of amplification primers 131 of a first type, and in which the second set of capture primers comprises amplification primers of a second type that is different from the first type, in other examples (such as described further below) a mixture of different types of capture primers may be located in the first region and/or in the second region.

Although FIG. 1A illustrates an example in which the sets of capture primers 131, 141 are disposed on the same surface of substrate 100 as one another (that is, are coplanar) and separated from one another by a gap which lies along that same surface, it will be appreciated that any suitable substrate geometry may be used to separate the sets of capture primers from one another. Nonlimiting examples of alternative geometries will be described with reference to FIGS. 3, 4, 5A-5B, 6, 7, 8A-8B, 9A-9F, 10A-10E, 11, 12A-12B, 13-18, 19A-19B, 25A-25G, and 26A-26E. Nonlimiting examples of forming such alternative geometries are described elsewhere herein.

The first adapter of a polynucleotide from the fluid may be hybridized to a capture primer of the first set of capture primers. For example, referring now to FIG. 1B, first adapters 154 of polynucleotides 151 and 152 may hybridize to respective capture primers 131, while second adapter 155 of polynucleotide 153 may hybridize to a capture primer 141. In this regard, note that whether capture primers are referred to herein as being “first” or “second” is generally arbitrary. For example, for polynucleotides 151 and 152, primers 131 may be considered to be the “first” set of capture primers because that is where the adapters of those polynucleotides 151 and 152 initially hybridize, whereas for polynucleotide 153, primers 141 may be considered to be the “first” set of capture primers because that is where the adapter of that polynucleotide 153 initially hybridizes. First adapters 154 may be sufficiently complementary to capture primers 131 as to hybridize thereto at the temperature at which the operation illustrated in FIG. 1B is conducted. For example, first adapter 154 of polynucleotide 151 may form a relatively stable duplex 161 with a capture primer 131, and first adapter 154 of polynucleotide 152 may form a relatively stable duplex 162 with a different capture primer 131. Additionally, second adapters 155 may be sufficiently complementary to capture primers 141 as to hybridize thereto at the temperature at which the operation illustrated in FIG. 1B is conducted. For example, second adapter 155 of polynucleotide 153 may form a relatively stable duplex 163 with a capture primer 141. Capture primers 131 and 141 may have any suitable sequences, e.g., that are orthogonal to one another. That is, capture primer 141 may be referred to as an orthogonal capture primer, while capture primer 131 may be referred to as a capture primer; or capture primer 131 may be referred to as an orthogonal capture primer, while capture primer 141 may be referred to as a capture primer. In one nonlimiting example, adapters 154 may include cP5, and adapters 155 may include cP7.

Based upon the hybridized polynucleotide being sufficiently long to bridge the gap, that polynucleotide may be amplified using the first set of capture primers and the second set of capture primers; and based upon the hybridized polynucleotide being insufficiently long to bridge the gap, that polynucleotide may not be amplified. For example, referring now to FIG. 1C, after the initial hybridizations described with reference to FIG. 1B, each of first, second, and third target polynucleotides 151, 152, 153 may be amplified, e.g., forming first amplicon 151′, second amplicon 152′, and third amplicon 153′ respectively. Following such amplification, the first, second, and third target polynucleotides 151, 152, 153 may be dehybridized in a manner such as illustrated in FIG. 1D, while first, second, and third amplicons 151′, 152′, 153′ remain covalently bound to substrate 100. Note that such dehybridization need not necessarily be performed. For example, instead of dehybridizing the first, second, and third target polynucleotides 151, 152, 153, such polynucleotides may remain hybridized to the substrate and may be further amplified using a strand invasion process such as known in the art and may be referred to as ExAmp. In such examples, however, note that polynucleotides that are sufficiently long to bridge gap A, and thus to access both primers 131 and primers 141, may be amplified in a manner similar to that as will now be described.

As illustrated in FIG. 1E, after the initial amplifications described with reference to FIGS. 1C-1D, the resulting amplicons may bend so as potentially to hybridize to a different type of capture primer on substrate 100. Amplicons that are sufficiently long to bridge gap A may be able to access the other type of capture primer and thus be amplified. For example, amplicon 151′ is too short to bridge gap A, and as such that amplicon's adapter may not be able to reach any of primers 141 so as to hybridize thereto. Similarly, amplicon 153′ is too short to bridge gap A, and as such that amplicon's adapter may not be able to reach any of primers 131 so as to hybridize thereto. Such inability to hybridize may inhibit amplification of amplicons 151′ and 153′, e.g., may inhibit a polymerase (not specifically illustrated) from being able to form an amplicon of using a capture primer 141 or 131, respectively, as a primer for such amplification.

For example, based upon amplicon 152′ being sufficiently long to bridge gap A (i.e., based upon polynucleotide 152 being sufficiently long to bridge gap A), amplicon 152′ may bridge the gap such that adapter 155 of amplicon 152′ hybridizes with one of primers 141 to form duplex 164. The hybridization of the second adapter 155 of amplicon 152′ to capture primer 141 may promote amplification of second amplicon 152′, and thus promote further amplification of target polynucleotide 152. For example, duplex 164 resulting from hybridization between adapter 155 of amplicon 152′ and capture primer 141 may have a melting temperature (Tm) of greater than about 40° C., e.g., of greater than about 45° C., or of greater than about 50° C., or of greater than about 55° C., or of greater than about 60° C., or of greater than about 65° C., or of greater than about 70° C. As such, at the reaction temperature, duplex 166 (as well as duplex 162 described with reference to FIG. 1B) is likely to be formed for a sufficient amount of time for a polymerase (not specifically illustrated) to begin to form an amplicon of amplicon 152′ using capture primer 141 as a primer for such amplification.

As such, a target polynucleotide (or amplicon thereof) that may be considered to be “sufficiently long” is one that may hybridize to a capture primer on one side of gap A, and may hybridize to another capture primer (e.g., an orthogonal capture primer) on the other side of gap A. It will be appreciated that the longer the target polynucleotide (or amplicon), the more easily it may be able to hybridize to a capture primer on one side of gap A and to bridge across gap A to hybridize to another capture primer (e.g., orthogonal capture primer) on the other side of gap A. Conversely, the larger gap A is, the longer the polynucleotide will need to be in order to bridge the gap and be amplified. As such, the gap may be sized such that a polynucleotide having at least a threshold length is amplified, while a polynucleotide having a length less than the threshold may not be amplified.

In some examples, the approximate relationship between the length A of the gap and the number of bases (B) in the polynucleotide that can bridge that gap may be expressed as BP=A/0.34 nm. Accordingly, based upon the polynucleotide being about A/0.34 nm bases long or greater, the polynucleotide is amplified, and based upon the polynucleotide being less than about A/0.34 nm bases long, the polynucleotide is not amplified. Illustratively, for a gap A that is about 100 nm across, a polynucleotide would need to have about 294 base pairs or more to sufficiently bridge the gap and thus to be amplified, while a polynucleotide having less than about 294 base pairs is not amplified. Alternatively, for a gap A that is about 150 nm across, a polynucleotide would need to have about 441 base pairs or more to sufficiently bridge the gap and thus to be amplified, while a polynucleotide having less than about 441 base pairs is not amplified. Alternatively, for a gap A that is about 200 nm across, a polynucleotide would need to about 588 base pairs or more to sufficiently bridge the gap and thus to be amplified, while a polynucleotide having less than about 588 base pairs is not amplified. Alternatively, for a gap A that is about 250 nm across, a polynucleotide would need to have about 735 base pairs or more to sufficiently bridge the gap and thus to be amplified, while a polynucleotide having less than about 735 base pairs is not amplified.

Put another way, the size of the gap may be selected so as to exclude amplification of polynucleotides with lengths that are less than the size of the gap. As such, to select for polynucleotides with lengths of at least 400 base pairs, a gap length of about 136 nm may be used; to select for polynucleotides with lengths of at least 500 base pairs, a gap length of about 170 nm may be used; to select for polynucleotides with lengths of at least 600 base pairs, a gap length of about 204 nm may be used; to select for polynucleotides with lengths of at least 700 base pairs, a gap length of about 238 nm may be used; to select for polynucleotides with lengths of at least 800 base pairs, a gap length of about 272 nm may be used; to select for polynucleotides with lengths of at least 900 base pairs, a gap length of about 306 nm may be used, and so on. As such, in some examples a gap length in the range of about 100 nm to about 1000 nm may be used, illustratively in the range of about 200 nm to about 1000 nm, e.g., about 200 nm to about 600 nm, or about 100 nm to about 400 nm.

An amplified cluster may be formed for polynucleotide(s) that are sufficiently long to bridge the gap A. For example, FIG. 1F illustrates the composition of FIG. 1E following another amplification operation. It may be seen that the composition includes an additional amplicon 152″ of amplicon 152′. Such additional amplicon may be hybridized to amplicon 152′. In comparison, the composition may not include any further amplicons of amplicons 151′, 153′, e.g., because those amplicons are too short to bridge across gap A to reach the other type of capture primer needed for amplification. The amplification operation may be repeated any suitable number of times so as to generate further amplicons of amplicons 152′, 152″ illustrated in FIG. 1G, which can bridge across gap A to reach the other type of capture primer needed for amplification. For example, as shown in FIG. 1H, adapter 155 of amplicon 152′ may hybridize to capture primer 141, and adapter 154 of amplicon 152″ may hybridize with a capture primer 131. As shown in FIG. 1I, following similar additional amplification operations, additional amplicons 152″ then may be formed. If amplification operations are repeated until the first and second substrate regions are full, both adapters of the resulting amplicons may not necessarily be hybridized to corresponding capture primers or orthogonal capture primers, and as such the amplicons may extend linearly away from the substrate as illustrated in FIG. 1I.

Amplification operations may be formed any suitable number of times so as to substantially fill both of the first and second substrate regions with at least functionally monoclonal clusters, and in some examples substantially monoclonal clusters, e.g., with amplicons of target polynucleotide 152. For example, amplicons within each of the first and second substrate regions each may include at least about 60% amplicons of one selected target polynucleotide, or at least about 70% amplicons of one selected target polynucleotide, or at least about 80% amplicons of one selected target polynucleotide, or at least about 90% amplicons of one selected target polynucleotide, or at least about 95% amplicons of one selected target polynucleotide, or at least about 98% amplicons of one selected target polynucleotide, or at least about 99% amplicons of one selected target polynucleotide, or about 100% amplicons of one selected target polynucleotide.

In some examples, certain capture primers and orthogonal capture primers may include non-nucleotide moieties. Such non-nucleotide moieties may include, but are not limited to, excision moieties via which a portion of the capture primers selectively may be removed. For example, capture primers 131 optionally may include excision moieties and/or capture primers 141 optionally may include excision moieties (excision moieties not specifically shown in FIGS. 1A-1I). The excision moieties of capture primers 131 may be of the same type as, or a different type than, the excision moieties of capture primers 141. The excision moieties may be located at any suitable position along the length of any suitable primer(s) and may be, but need not necessarily be, the same type of excision moiety as one another. Following a desired number of amplification operations such as described with reference to FIGS. 1A-1I, portions of capture primers 131 may be removed by reacting a suitable enzyme or reagent with the respective excision moieties, and/or portions of capture primers 141 may be removed by reacting a suitable enzyme or reagent with the respective excision moieties. The enzyme or reagent used with the excision moieties of capture primers 131 may be the same as, or different than, the enzyme or reagent used with excision moieties of capture primers 141. Illustrative examples in which one or primers 131 and/or primers 141 include excision moieties are described elsewhere herein.

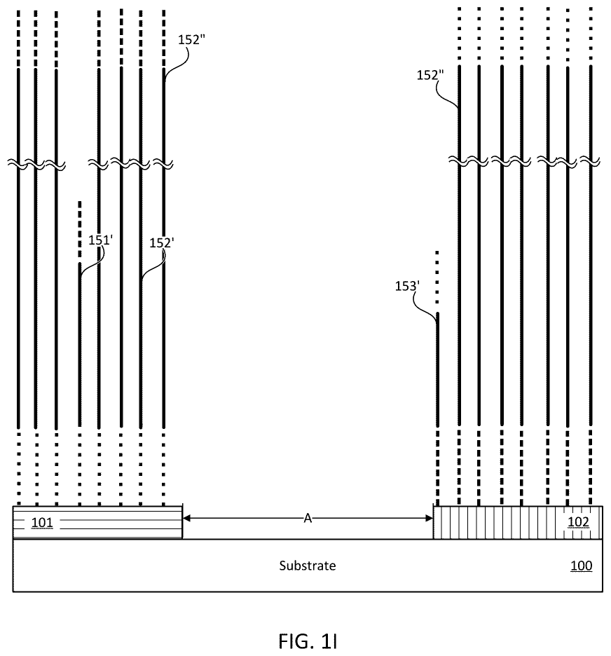

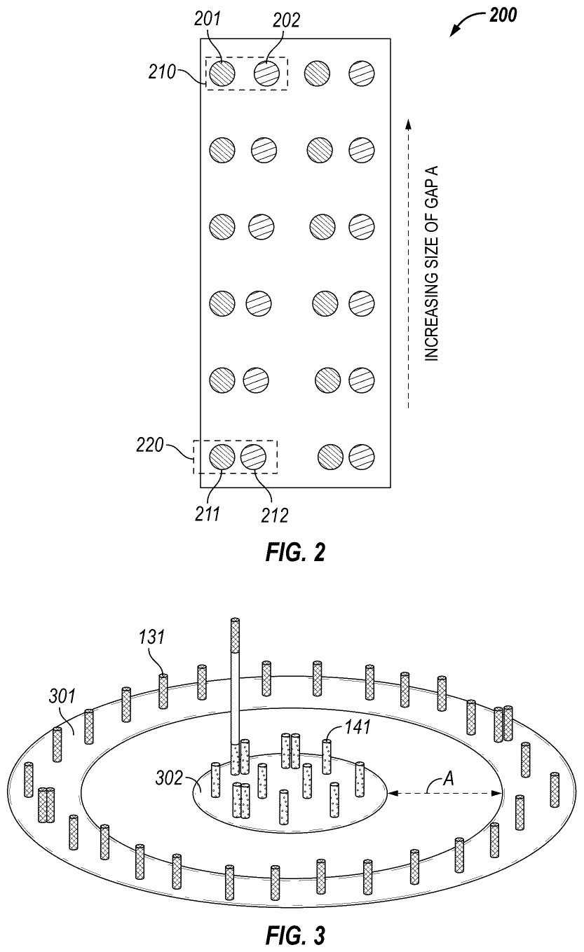

As noted further above, a wide variety of substrate geometries are compatible with operations such as described with reference to FIGS. 1A-1I. For example, FIGS. 2, 3, 4, 5A-5B, 6, 7, 8A-8B, 9A-9F, and 10A-10E schematically illustrate additional example structures and operations for capturing and amplifying polynucleotides in a manner such as described with reference to FIGS. 1A-1I.

Turning now to FIG. 2, an array of structures on a substrate 200 is illustrated in which the size of gap A is different for different structures, so as to selectively amplify polynucleotides of different lengths than one another. For example, structure 210 includes first region 201 and second region 202 separated from one another by a gap A of a first size, while structure 220 includes first region 211 and second region 212 separated from one another by a gap A of a second size which is smaller than the size of gap A for pair 220. Any suitable number of pairs, with any suitable size of respective gaps, may be included in the array. Each such pair may be used in a manner such as described with reference to FIGS. 1A-1I.

Although FIGS. 1A-11 and 2 illustrate structures in which first and second regions of the substrate are disposed adjacent to one another, with a gap therebetween, other configurations may be used. For example, FIG. 3 illustrates a configuration in which a first region 301 of the substrate includes an aperture, and the second region 302 of the substrate is disposed within the aperture. As such, first region 301 may surround second region 302. The distance between the inner surface of first region 301 and the outer surface of second region 302 provides a gap A which may be used in a similar manner as described with reference to FIGS. 1A-1I. Although FIG. 3 suggests that the first and second regions 301, 302 are circular, other geometrical shape(s) suitably may be used. Additionally, note that the designation of a region as being “first” or “second” is arbitrary, and as such a second region equivalently may be considered to surround a first region.

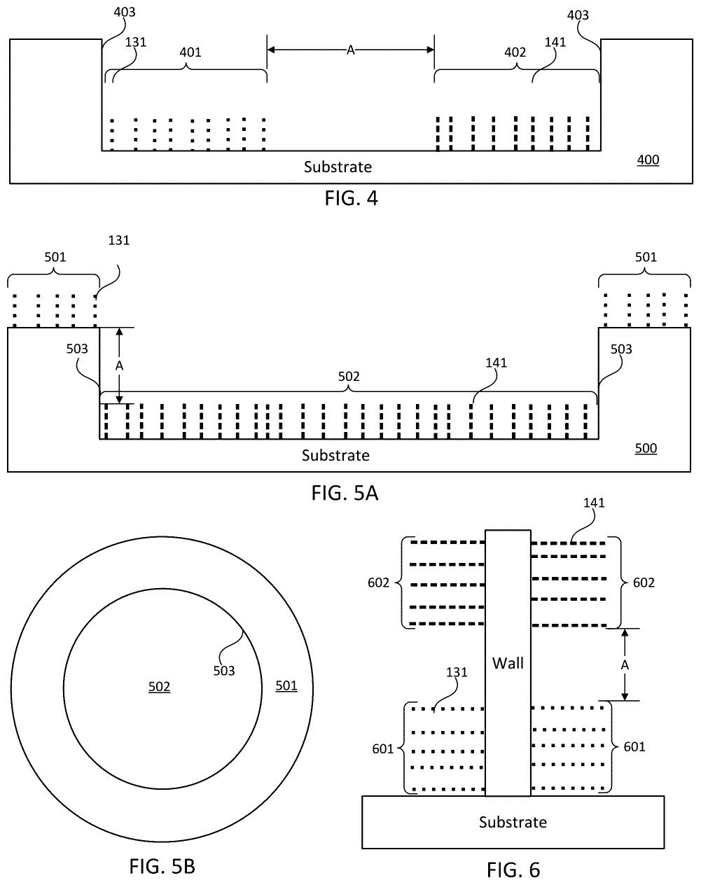

Although FIGS. 1A-11, 2, and 3 may suggest that the first and second regions may be coplanar and both may be located on the upper surface of a substrate, such regions may be located at any suitable locations on or within the substrate, and need not be coplanar. In some examples, the first region and the second region are vertically separated from one another. For example, FIG. 4 illustrates a configuration in which first region 401 and second region 402 are both located within a recess of substrate 400. Vertical sidewall(s) 403 may form outer boundar(ies) of first region 401 and/or second region 402. Gap A may be defined similarly as described with reference to FIGS. 1A-11 or FIG. 2. Or, for example, FIGS. 5A-5B illustrate a configuration in which first region 501 is disposed on a raised surface of the substrate 500, and second region 502 is located within a recess of substrate 500, in which FIG. 5A provides a cross-sectional view and FIG. 5B is a simplified plan view. At least partially vertical sidewall(s) 503 may form an outer boundary of second region 402. In this example, the gap between first region 501 and second region 502 may be in the vertical dimension, rather than in the horizontal dimension as illustrated in FIGS. 1A-11, 2, and 3. Or, for example, the first region and the second region may be vertically separated from one another. FIG. 6 illustrates a configuration in which first region 601 is disposed at a first location on a vertical wall of a substrate, and second region 602 is disposed at a second location on the vertical wall that is spaced apart from the first region 601 by gap A. In the examples illustrated in FIGS. 3-6, capture primers 131 and 141 may be used in a manner similar to that described with reference to FIGS. 1A-1I.

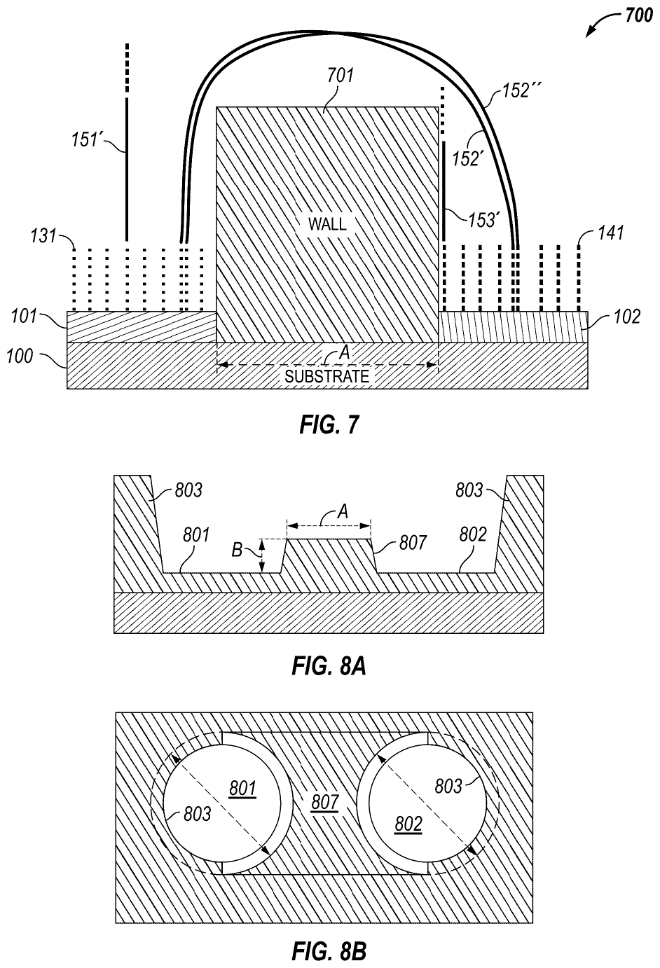

In these examples or in other examples, walls may be disposed between the first and second regions of the substrate so as to further enhance or control selectivity for amplifying relatively long polynucleotides. Illustratively, structure 700 shown in FIG. 7 includes substrate 100, first and second hydrogels 101, 102, capture primers 131 coupled to a first region of the substrate, and capture primers 141 coupled to a second region of the substrate, wherein the first region is spaced apart from the second region by gap A in a similar manner as described with reference to FIGS. 1A-1E. As illustrated in FIG. 7, structure 700 also includes wall 701 disposed within the gap A between the first region and the second region. Polynucleotides that are sufficiently long to bridge the gap A between the first region (containing capture primers 131) and the second region (containing capture primers 141), and also sufficiently long to pass over wall 701, may be amplified, while polynucleotides that are not sufficiently long to bridge gap A and pass over wall 701 substantially may not be amplified. Accordingly, by controlling the size of gap A and/or the size of wall 701, amplification of polynucleotides over a threshold length may be permitted while amplification of polynucleotides less than the threshold length may be inhibited. In the nonlimiting example illustrated in FIG. 7, amplicons 151′ and 153′ are too short to pass over wall 701 and thus may not be amplified, while amplicon 152′ is sufficiently long to pass over wall 701 and thus may be amplified in a similar manner as described with reference to FIGS. 1A-11. In this example, gap A has the same size in the illustrated dimension as does wall 701, but it should be appreciated that gap A may be larger in the illustrated dimension than wall 701.

The use of a wall, such as described with reference to FIG. 7, may be compatible with a wide variety of configurations in a similar manner as described with reference to FIGS. 3-6. For example, For example, FIGS. 8A-8B, in which FIG. 8A provides a cross-sectional view and FIG. 8B is a simplified plan view, illustrates a configuration in which the first region (where primers 131 may be coupled) is located within a first recess 801 of the substrate, wherein the second region where primers 141 may be coupled) is located within a second recess 802 of the substrate, and wherein wall 807 divides the first recess from the second recess. At least partially vertical sidewall(s) 803 may form outer boundar(ies) of first region 801 and/or second region 802, while wall 807 may form inner boundar(ies) of first region 801 and/or second region 802. Gap A may be defined similarly as described with reference to FIG. 7, e.g., gap A may have the same size in the illustrated dimension as does wall 801. The height B of wall 801 may be selected in a manner such as described with reference to FIG. 7.

Although FIGS. 1A-1I illustrate an example in which two different types of capture primers are used, any suitable number of capture primers may be used. FIGS. 9A-9F schematically illustrate additional example structures and operations for capturing and amplifying polynucleotides using first and second sets of capture primers that are spaced apart from one another by a gap. In a manner similar to FIGS. 1A-1I, the first set of capture primers comprises a first type of capture primer, and the second set of capture primers comprises a second type of capture primer. However, the first and second sets of capture primers may be configured differently than in the example of FIG. 1A.

Referring now to FIG. 9A, structure 900 may be contacted with fluid 90. Fluid 90 may include target polynucleotides having a variety of lengths, e.g., polynucleotide fragments that are generated using commercially available fragmentation or tagmentation techniques, using DNA or RNA that it is desired to sequence. Illustratively, and in a manner similar to that described with reference to FIG. 1A, fluid 90 may include polynucleotide 151 which is of intermediate length, polynucleotide 152 which is relatively long, and polynucleotide 153 which is relatively short. However, while the polynucleotides within fluid 10 of FIG. 1A may be single-stranded, the polynucleotides within fluid 90 are substantially double-stranded. For example, polynucleotide 151 may be hybridized to substantially complementary strand 951′ to form double-stranded polynucleotide 151, 951′; polynucleotide 152 may be hybridized to substantially complementary strand 952′ to form double-stranded polynucleotide 152, 952′; and polynucleotide 153 may be hybridized to substantially complementary strand 953′ to form double-stranded polynucleotide 153, 953′. Although only three double-stranded polynucleotides are illustrated in FIG. 9A, it will be appreciated that fluid 90 may include thousands, or even millions, of double-stranded fragments, many of which may be of different lengths than one another.

Each of the polynucleotides 151, 152, 153 (and their respective substantially complementary strands 951′, 952′, 953′) may include first and second adapters, e.g., first adapter 154 and second adapter 155 which may be configured used in a manner such as described with reference to FIG. 1A, except that the adapters of the complementary strands also include adapter sequences that are complementary to 154 and 155, respectively; as such, these polynucleotides and their adapters may be referred to herein as being “double-stranded” in examples such as illustrated in FIG. 9A and other similar examples. Additionally, in the example illustrated in FIG. 9A, polynucleotides 151, 152, 153 optionally may further include one or more additional adapters, e.g., seeding adapters 956 which may be coupled to adapter 154 and/or adapter 155, for example via a respective linker 958. However, note that while seeding adapters 965 respectively may be coupled to polynucleotides 151, 152, and 153, the complementary strands 951′, 952′, 953′ may not include the complements of such seeding adapters. Accordingly, the seeding adapters 956 may be single-stranded, and thus available to hybridize with a substantially complementary seeding adapter in a manner such as now will be described. In comparison, adapters 154 and 155 are double-stranded and thus unavailable to hybridize with capture primers on the substrate until after certain processing steps are performed, as will be explained below.

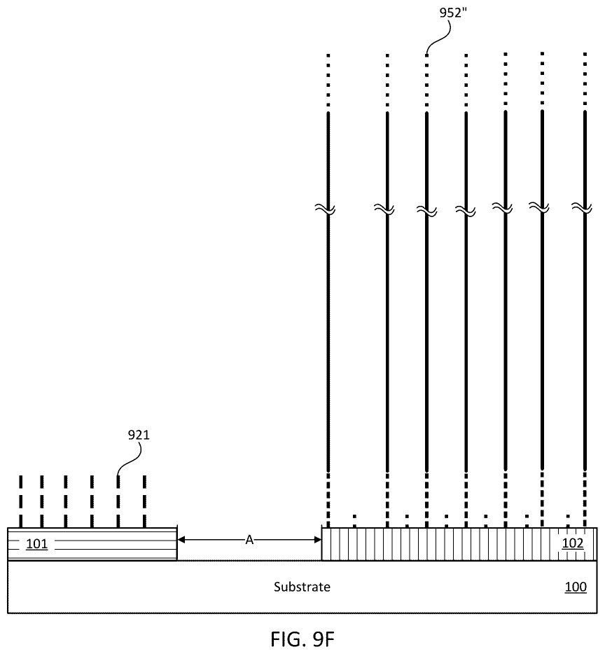

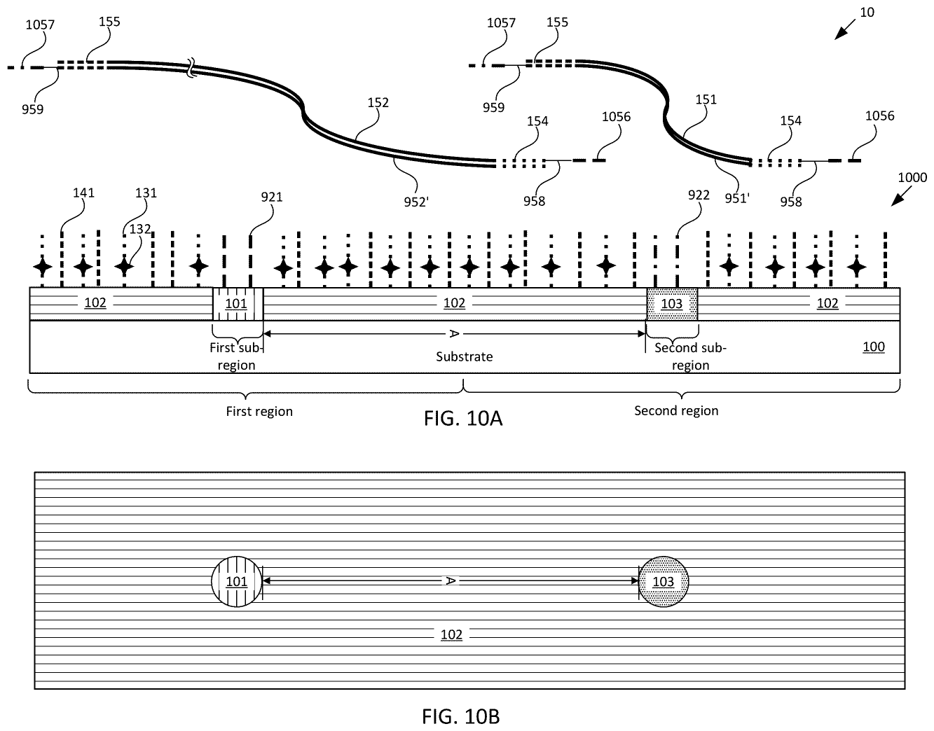

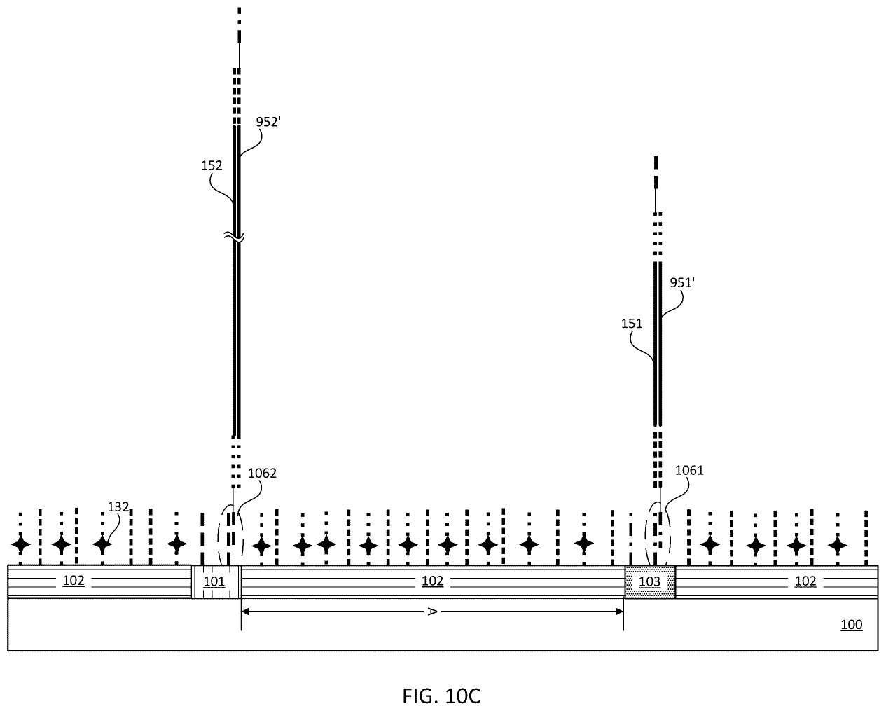

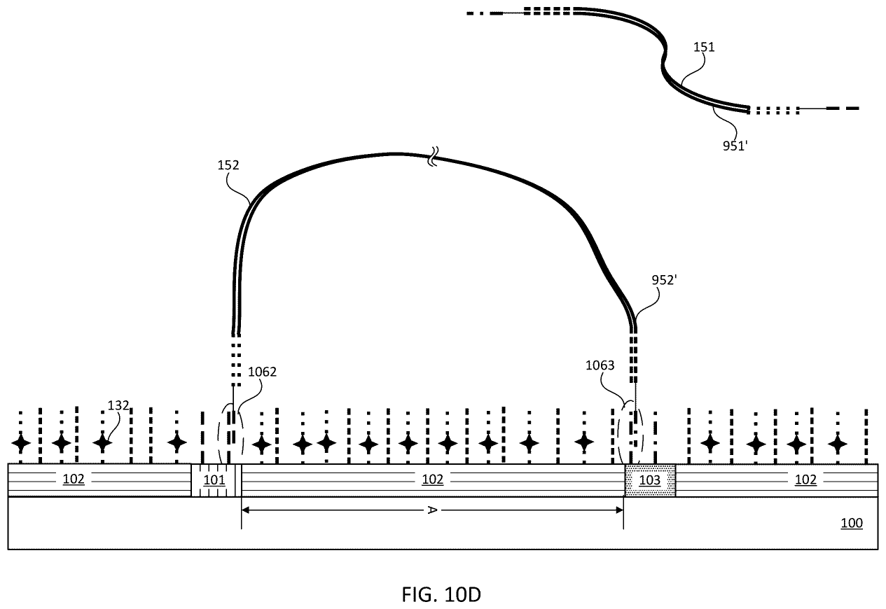

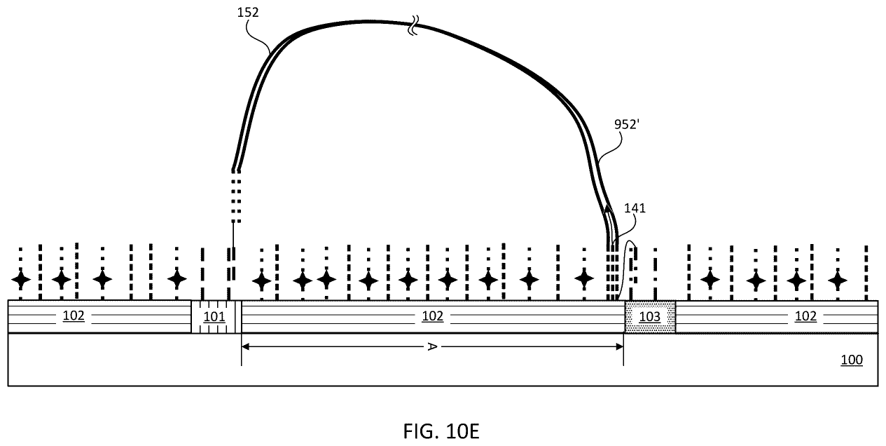

As illustrated in FIG. 9A, and in a manner similar to that described with reference to FIG. 1A, structure 900 may include substrate 100 including a first region and a second region spaced apart from one another by a gap (A) of at least 100 nm. Nonlimiting examples of gap sizes, and other geometrical configurations, are provided elsewhere herein. For examples in which a wall is provided between the first and second regions of the substrate, example wall sizes and gap sizes are provided elsewhere herein. A set of seeding primers 921 may be coupled to the first region of the substrate, and a mixture of capture primers 131 and capture primers 141 may be coupled to the second region of the substrate. Illustratively, the seeding primers 921 may be coupled to a first hydrogel 101 that is disposed on the first region of the substrate 100, and the sets of capture primers 131, 141 may be coupled to a second hydrogel that is disposed on the second region of the substrate 100. Capture primers 131 and 141 may have any suitable sequences, e.g., that are orthogonal to one another, as described above, and in some examples respectively may include P5 and P7 primers. Seeding primer 921 may have a sequence that is orthogonal to the sequences of capture primers 131 and 141. Optionally, seeding primer 921 may have approximately the same length as capture primers 131 and 141. Alternatively, in a manner such as described in greater detail below with reference to FIGS. 10A-10E, 22A-22C, 23A-23F, 24A-24C, 25A-25G, and 26A-26E, seeding primer 921 and/or seeding adapter 956 may be modified such that the resulting duplex may have a sufficiently low melting temperature to reduce the likelihood of multiple seeding events occurring in a given region of the substrate. Optionally, seeding primer 921 may have the sequence PX, and seeding adapter 956 may have the sequence cPX.

Although FIG. 9A illustrates an example in which the set of seeding primers 921 and mixture of capture primers 131, 141 are disposed on the same surface of substrate 100 as one another (that is, are coplanar) and separated from one another by a gap which lies along that same surface, it will be appreciated that any suitable substrate geometry may be used to separate the various sets of primers from one another. Nonlimiting examples of alternative geometries are described with reference to FIGS. 3, 4, 5A-5B, 6, 7, 8A-8B, 10A-10E, 11, 12A-12B, 13-18, 19A-19B, 25A-25G, and 26A-26E. Nonlimiting examples of forming such alternative geometries are described elsewhere herein.

Referring now to FIG. 9B, seeding adapters 956 of polynucleotides 151, 152, and 153 may hybridize to respective seeding adapters 921 to form respective duplexes 961, 962, and 963. Because adapters 154 and 155 are double-stranded, e.g., already hybridized to their counterparts for complementary strands 951′, 952′, 953′, adapters 154 and 155 are not available to hybridize to adapters 131 and 141 respectively. Accordingly, the majority of seeding, and in some examples substantially all seeding, of the polynucleotides may be expected to be via hybridization between adapters 956 and primers 921 in the first region of the substrate.

Based upon the hybridized polynucleotide being sufficiently long to bridge the gap, that polynucleotide may be amplified using the first set of capture primers and the second set of capture primers; and based upon the hybridized polynucleotide being insufficiently long to bridge the gap, that polynucleotide may not be amplified. For example, after the initial hybridizations described with reference to FIG. 9B, polynucleotide 152 may be amplified, e.g., forming amplicon 152′. More specifically, polynucleotides that are sufficiently long to bridge gap A, and thus to access both primers 131 and primers 141 from the first region of the substrate, may be amplified. As illustrated in FIG. 9C, after the initial hybridizations described with reference to FIG. 9B, the double-stranded polynucleotides may bend so as potentially to hybridize to a different type of capture primer on substrate 100. Double-stranded polynucleotides that are sufficiently long to bridge gap A may be able to access capture primers 131 and 141 and thus be amplified. For example, double-stranded polynucleotide 151, 951′ is too short to bridge gap A, and as such that polynucleotide's free adapter may not be able to reach any of primers 131 or 141 so as to hybridize thereto. Similarly, double-stranded polynucleotide 153, 953′ is too short to bridge gap A, and as such that polynucleotide's free adapter may not be able to reach any of primers 131 or 141 so as to hybridize thereto. Such inability to hybridize may inhibit amplification of polynucleotides 151 and 153, e.g., may inhibit a polymerase (not specifically illustrated) from being able to form an amplicon of using a capture primer 131 or 141, respectively, as a primer for such amplification.

In comparison, based upon double-stranded polynucleotide 152, 952′ being sufficiently long to bridge gap A, the polynucleotide may bridge the gap such that adapter 155 double-stranded polynucleotide 152, 952′ hybridizes with one of primers 141 to form duplex 664 using a process that may be referred to as “strand invasion” and may be promoted using a recombinase (not specifically illustrated in FIG. 9C). An amplified cluster may be formed for polynucleotide(s) that are sufficiently long to bridge the gap A. For example, FIG. 9D illustrates the composition of FIG. 9C during recombinase-promoted extension of the primer 141 to which double-stranded polynucleotide 152, 952′ hybridizes to form amplicon 952″ which is covalently coupled to hydrogel 102 in the second region of the substrate. Amplicon 952″ repeatedly may be further amplified using strand invasion. For example, it may be seen that the composition of FIG. 9E includes amplicon 952″ and a plurality of additional amplicons of amplicon 952″ that are formed using a mixture of capture primers 131 and 141, which are located in the second region of the substrate, for the amplification. If amplification operations are repeated until the second substrate region is substantially full, both adapters of the resulting amplicons may not necessarily be hybridized to corresponding capture primers or orthogonal capture primers, and as such the amplicons may extend linearly away from the substrate as illustrated in FIG. 9E. In the example illustrated in FIG. 9E, capture primers 131 may include excision moieties 132 which may be used to remove amplicons that are oriented in a selected direction, so that the remaining amplicons are oriented substantially in the same direction as one another in a manner such as illustrated in FIG. 9F.

For further details regarding seeding and amplification operations using strand invasion and seeding adapters and seeding primers that are orthogonal to capture adapters and capture primers, see International Patent Application No. PCT/US2022/053002, filed on Dec. 15, 2022 and entitled “Orthogonal Hybridization,” the entire contents of which are incorporated by reference herein.

As such, in a manner similar to that described with reference to FIGS. 1A-1I, a target polynucleotide that may be considered to be “sufficiently long” is one may hybridize to a capture primer (e.g., seeding primer 921) on one side of gap A, and may hybridize to another capture primer (e.g., a different type of capture primer) on the other side of gap A. In the examples described with reference to FIGS. 9A-9F, a plurality of different polynucleotide sizes may be seeded (hybridized to seeding primers 921). Those polynucleotides which are sufficiently long to cross gap A may subsequently be amplified. Because a mixture of amplification primers 131, 141 is in the second region of the substrate, on the other side of the gap, once the polynucleotide has crossed the gap neither it nor its amplicons need to cross the gap again in order to be amplified.

As noted above with reference to FIG. 9A, linker 958 may be provided between capture adapter 154 and seeding adapter 956. In some examples, linker 958 may be or include one or more of: a carbon-containing chain with a formula (CH₂)ₙ wherein “n” is from 1 to about 1500, for example less than about 1000, preferably less than 100, e.g. from 2-50, particularly 5-25; polyethylene glycol (PEG); or iSp9 (Spacer 9) which is a triethylene glycol spacer that can be incorporated at the 5′-end or 3′-end of an oligo or internally. Linkers formed primarily from chains of carbon atoms or from PEG may be modified so as to contain functional groups which interrupt the chains. Examples of such groups include ketones, esters, amines, amides, ethers, thioethers, sulfoxides, sulfones. Separately or in combination with the presence of such functional groups, groups such as alkene, alkyne, aromatic or heteroaromatic moieties, or cyclic aliphatic moieties (e.g., cyclohexyl) may be included. Cyclohexyl or phenyl rings may, for example, be connected to a PEG or (CH₂)ₙ chain through their 1- and 4-positions. Other linkers are envisaged which are based on nucleic acids or monosaccharide units (e.g., dextrose). It is also within the scope of this disclosure to utilise peptides as linkers. A variety of other linkers may be employed. The linker preferably is stable under conditions under which the polynucleotides are intended to be used subsequently, e.g., conditions used in DNA amplification. The linker should also be such that it is not by-passed by DNA polymerases, terminating DNA polymerization before copying the capture moiety sequence (if it is nucleotide based such as a PX′ sequence). This allows for PX′ to remain single-stranded and available for hybridization at all times.

In some examples, the double-stranded polynucleotides may include an additional single-stranded seeding adapter which may be used to provide further size selectivity during the seeding process. For example, FIGS. 10A-10E schematically illustrate example structures and operations for capturing and amplifying polynucleotides using first and second sets of seeding primers that are spaced apart from one another by a gap. Turning now to FIGS. 10A, the polynucleotides (of which only double-stranded polynucleotide 151, 951′ and double-stranded polynucleotide 152, 952′ are illustrated for simplicity) may include double-stranded first adapter 154 and double-stranded second adapter 155 such as described with reference to FIGS. 1A-11; a third adapter 1056 (first single-stranded seeding adapter) which may be shorter than amplification primers 131 and/or 141 and may be shorter than seeding adapter 956 described with reference to FIGS. 9A-9F; and a fourth adapter 1057 (second single-stranded seeding adapter which may be shorter than amplification primers 131 and/or 141, may be shorter than seeding adapter 956 and has an orthogonal sequence to that of third adapter 1056). In the example illustrated in FIG. 10A, single-stranded adapter 1056 is coupled to adapter 154 of polynucleotide 151 or 152 via linker 958, and single-stranded adapter 1057 is coupled to adapter 155 of complementary polynucleotide 951′ or 952′ via linker 959, in a manner similar to that described with reference to FIGS. 9A-9F. Structure 1000 illustrated in FIG. 10A may be contacted with fluid 10 containing a mixture of different lengths of such polynucleotides.

Structure 1000 may include a first set of capture primers (e.g., amplification primers 131 and 141) in the first region, and a second set of primers (e.g., additional amplification primers 131 and 141) in the second region. Structure 1000 further may include third and fourth sets of capture primers. For example, the first region also may include seeding primers 921 (third set) which are at least partially complementary to seeding adapters 1056, and the second region may include seeding primers 922 (fourth set) which are at least partially complementary to seeding adapters 1057. Seeding primers 921 may be located within a first sub-region which is located within the first region, and seeding primers 922 may be located within a second sub-region which is located within the second region. In some examples, seeding primers 921 are coupled to hydrogel 101 located in the first sub-region, seeding primers 922 are coupled to hydrogel 103 located in the second sub-region, a subset of amplification primers 131 and 141 are coupled to hydrogel 102 located in the first region, and a different subset of amplification primers 131 and 141 are coupled to hydrogel 102 in the second region. FIG. 10B illustrates a plan view of an example arrangement of hydrogels 101, 102, and 103, in which hydrogels 101 and 102 are surrounded by hydrogel 103. In a manner such as illustrated in FIGS. 10A-10B, the region containing seeding primers 921 (coupled to hydrogel 101) and the region containing seeding primers 922 (coupled to hydrogel 103) are spaced apart from one another by a gap A.

Note that use of the terms “first,” “second,” “third,” or “fourth” here and elsewhere herein in are arbitrary. As such, the set of seeding primers 921 may be considered to be a first set of capture primers, and the set of seeding primers 922 may be considered to be a second set of capture primers. Additionally, or alternatively, the sub-region to which the set of seeding primers 921 is coupled may be considered to be a first region of the substrate, and the sub-region to which the set of seeding primers 922 is coupled may be considered to be a second region of the substrate. Additionally, or alternatively, the first and second regions of the substrate within which the mixtures of amplification primers 131, 141 are disposed may be contiguous with each other, and indeed may be substantially indistinguishable from one another other than for their respective locations on the substrate.

Polynucleotides that are sufficiently long to bridge the gap A may be amplified using seeding primers 921 and seeding primers 922, even though the seeding primers themselves may not necessarily provide a primer that may be extended in an amplification operation. In comparison, polynucleotides that are insufficiently long to bridge the gap A may not be amplified. For example, as illustrated in FIG. 10C, seeding adapters 1057 of certain double-stranded polynucleotides (here, of double-stranded polynucleotide 151, 951′) may hybridize to respective seeding primers 922, and seeding adapters 1056 of certain other double-stranded polynucleotides (here, of double-stranded polynucleotide 152, 952′) may hybridize to respective seeding primers 921. As noted above, in the nonlimiting example illustrated in FIG. 10A, seeding adapters 1056 and 1057 each may be shorter than seeding adapter 956. As such, the melting temperature (Tm) of the duplex 1062 between seeding adapter 1056 and seeding primer 921 is lower than that of the duplex 961, 962, or 963 between seeding adapter 956 and seeding primer 921. Similarly, the melting temperature (Tm) of the duplex 1061 between seeding adapter 1057 and seeding primer 922 is lower than that of the duplex 961, 962, or 963 between seeding adapter 956 and seeding primer 921. Therefore, duplexes 1061 and 1062 are less stable than duplexes 961, 962, and 963. This lower stability may be used to enhance monoclonality and/or to enhance size selectivity for double-stranded polynucleotides that are long enough to bridge gap A as compared to double-stranded polynucleotides that are not long enough to bridge gap A.

More specifically, the melting temperature of the duplex 1061 may be selected, e.g., by selecting a suitable length of seeding adapter 1056, such that duplex 1061 alone is insufficiently stable to retain the double-stranded polynucleotide at the substrate for sufficient time to amplify the double-stranded polynucleotide using amplification adapters 154, 155. Similarly, the melting temperature of duplex 1062 may be selected, e.g., by selecting a suitable length of seeding adapter 1057, such that duplex 1062 alone is insufficiently stable to retain the double-stranded polynucleotide at the substrate for sufficient time to amplify the double-stranded polynucleotide using amplification adapters 154, 155. For example, duplex 1061 and/or 1062 may have a melting temperature (Tm) which is at least about 1° C. lower than a melting temperature of a duplex between amplification adapter 154 and amplification primer 131 and/or of a duplex between amplification adapter 155 and amplification primer 141, e.g., at least about 2° C. lower, or at least about 3° C. lower, or at least about 4° C. lower, or at least about 5° C. lower, or at least about 10° C. lower, or at least about 15° C. lower, or at least about 20° C., or at least about 30° C. lower, than a melting temperature of a duplex between amplification adapter 154 and amplification primer 131 and/or of a duplex between amplification adapter 155 and amplification primer 141. Additionally, or alternatively, duplex 1061 and/or 1062 may have a melting temperature (Tm) which is at least about 1° C. lower than the temperature at which duplex 1061 is formed, e.g., at least about 2° C. lower, or at least about 3° C. lower, or at least about 4° C. lower, or at least about 5° C. lower, or at least about 10° C. lower, or at least about 15° C. lower, or at least about 20° C. lower, than the temperature at which duplex 1061 is formed.

In comparison, a duplex between amplification adapter 154 and amplification primer 131, and/or a duplex between amplification adapter 155 and amplification primer 141, may have a melting temperature (Tm) which is at least about 1° C. higher than the temperature at which such duplex is formed, e.g., at least about 2° C. higher, or at least about 3° C. higher, or at least about 4° C. higher, or at least about 5° C. higher, or at least about 10° C. higher, or at least about 15° C. higher, or at least about 20° C. higher, than the temperature at which such duplex is formed. In one nonlimiting example in which the duplexes are formed at a temperature of around 50° C., duplexes 1061 and 1062 may have a melting temperature of about 35-50° C. (illustratively about 40-50° C.) and duplexes between amplification adapter 154 and amplification primer 131, and between amplification adapter 155 and amplification primer 141, may have a melting temperature of about 55-65° C. (illustratively about 57-62° C.).

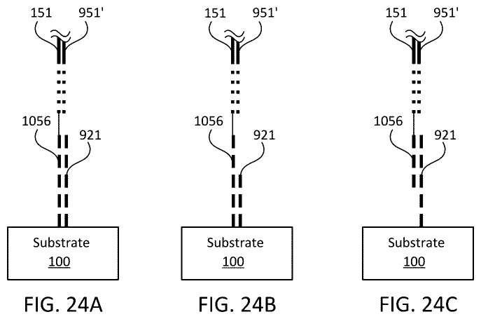

It will be appreciated that the melting temperatures of duplexes 1061 and/or 1062 also, or alternatively, may be selected in a manner such as described further below with reference to FIGS. 24A-24C. For example, the melting temperatures of duplexes 1061 and/or 1062 may be selected via suitable co-selection of base pairs, for example reducing the number of G-C pairs in the duplexes as compared to a duplex between amplification adapter 154 and amplification primer 131, and/or as compared to a duplex between amplification adapter 155 and amplification primer 141. Additionally, or alternatively, the melting temperatures of duplexes 1061 and/or 1062 also, or alternatively, may be selected by reducing the lengths of seeding primers 921 and/or 922. However, it should be understood that the melting temperatures of duplexes 1061 and/or 1062 may be selected using any suitable combination of parameters, including but not limited to the lengths of either or both of the elements which hybridize to one another to form such duplexes, or the compositions of either or both of the elements which hybridize to one another to form such duplexes.

As illustrated in FIG. 10D, double-stranded polynucleotide 152, 952′ is sufficiently long to bridge gap A, such that single-stranded seeding adapter 1056 of polynucleotide 152 hybridizes to a primer 921 to form duplex 1062, and single-stranded seeding adapter 1057 of complementary polynucleotide 952′ hybridizes to a primer 922 to form duplex 1063. Note that duplex 1061 alone would be insufficient to retain double-stranded polynucleotide 152, 952′ at the substrate for enough time to amplify the polynucleotide. Similarly, duplex 1063 alone would be insufficient to retain double-stranded polynucleotide 152, 952′ at the substrate for enough time to amplify the polynucleotide. In comparison, duplexes 1061 and 1063 together are sufficient to retain double-stranded polynucleotide 152, 952′ at the substrate for enough time to amplify the polynucleotide using the amplification adapters 154, 155 and amplification primers 131, 141. In comparison, because duplex 1062 alone is insufficient to retain double-stranded polynucleotide 151, 951′ at the substrate for enough time to amplify it, and because double-stranded polynucleotide 151, 951′ is too short to reach the first sub-region and thus to form a second duplex using a seeding primer 921, double-stranded polynucleotide 151, 951′ may be released from the substrate and return to solution.

As illustrated in FIG. 10E, double-stranded polynucleotide 152, 952′ may be amplified at one end or at both ends using a strand invasion process such as described with reference to FIGS. 9A-9F (amplification at one end, using primer 141, being illustrated in FIG. 10E). In this regard, note that various elements illustrated in FIGS. 10A-10E may be flexible, and so can move to permit certain interactions between various elements. For example, although adapter 1057 may be hybridized to primer 922, the linker 959 between that adapter 1057 and adapter 155 may be flexible, and/or the hydrogel to which primer 141 is coupled may be flexible, so as to permit primer 141 to strand invade adapter 155 for amplification.

Accordingly, examples such as described with reference to FIGS. 10A-10E may provide enhanced size selectivity because those polynucleotides that are sufficiently long may be amplified, and may be hybridized to the substrate significantly longer than shorter polynucleotides, thus further increasing the relative likelihood of the longer polynucleotides being amplified. Such amplification may result in a structure similar to that illustrated in FIG. 9E, except that the amplicons may be distributed throughout the illustrated regions of the substrate in which hydrogel 102 is located. Excision moiety 132 may be used to selective remove amplicons which are oriented in a first direction, in a manner similar to that illustrated in FIG. 9F.

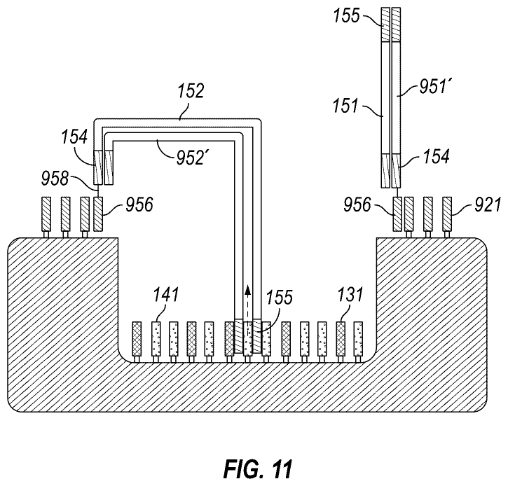

In a manner similar to that described with reference to FIGS. 1A-1I, the operations described with reference to FIGS. 9A-9F and 10A-10E are compatible with a variety of different geometries. FIGS. 11, 12A-12B, 13, 14, 15, 16 schematically illustrate additional example structures and operations for capturing and amplifying polynucleotides in a manner such as described with reference to FIGS. 1A-11, 9A-9F, 10A-10E, 22A-22C, 23A-23F, 25A-25G, and 26A-26E. Referring now to FIG. 11, an example structure is illustrated in which the seeding primers 921 described with reference to FIGS. 9A-9F are coupled to a raised surface of the substrate which is configured analogously as region 501 described with reference to FIGS. 5A-5B, and a mixture of capture primers 131, 141 is disposed within a recess of the substrate which is configured analogously as region 502 described with reference to FIGS. 5A-5B. As illustrated in FIG. 11, double-stranded polynucleotides 151, 951′ and 152, 952′ respectively include single-stranded seeding adapters 956 that can hybridize to seeding primers 921, adapters 154 that can hybridize to primers 131, adapters 155 that can hybridize to primers 141, and linkers 958 coupling seeding adapters 956 to amplification adapters 154 in a manner such as described with reference to FIGS. 9A-9F. When adapter 956 of double-stranded polynucleotide 151, 951′ is hybridized to a seeding primer 921 in the first region, that polynucleotide is too short for its adapters 155 to reach (and thus be strand invaded using) primers 141 in the second region and thus may not be amplified. In comparison, when adapter 956 of double-stranded polynucleotide 152, 952′ is hybridized to a seeding primer 921 in the first region, that polynucleotide is sufficiently long for its adapters 155 to reach a primer 141 in the second region and thus may be amplified using strand invasion in a manner such as described with reference to FIGS. 9A-9F.

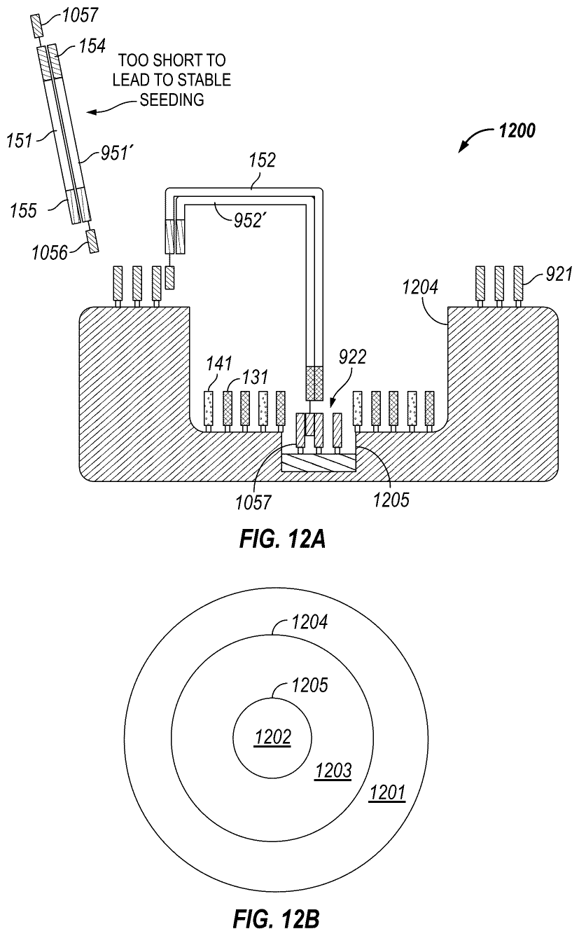

Although FIGS. 10A-10E illustrate an example in which the different regions of the substrate are coplanar that contain the different types of seeding and amplification primers, other geometries suitably may be used. For example, FIGS. 12A-12B illustrate cross-sectional and simplified plan views of an example structure 1200 in which seeding primers 921 are coupled to a raised surface 1201 of the substrate which is configured analogously as region 501 described with reference to FIGS. 5A-5B; a mixture of capture primers 131, 141 is disposed within a first recess 1203 of the substrate which is configured analogously as region 502 described with reference to FIGS. 5A-5B; and seeding primers 922 are disposed within a second recess 1202 of the substrate. Second recess 1202 may be surrounded by, and may be deeper than, recess 1203. Recess 1203 may be defined by a first sidewall 1204, and recess 1202 may be defined by second sidewall 1205. As illustrated in FIGS. 12A-12B, double-stranded polynucleotides 151, 951′ and 152, 952′ respectively include single-stranded seeding adapters 1056 that can hybridize to seeding primers 921, single-stranded seeding adapters 1057 that can hybridize to seeding primers 922, double-stranded adapters 154 that can hybridize to primers 131, and double-stranded adapters 155 that can hybridize to primers 141. When adapter 1056 of polynucleotide 151 is hybridized to a seeding primer 921 in the first region 1201, that polynucleotide is too short for its adapter 1057 to reach (and thus hybridize to) primers 922 in the second region and thus may not be amplified, for example because hybridization between adapter 1056 and seeding primer 921 may be relatively unstable. In comparison, when adapter 1056 of polynucleotide 152 is hybridized to a seeding primer 921 in the first region, that polynucleotide is sufficiently long for the adapter 1057 of complementary polynucleotide 952′ reach a primer 922 in region 1202. The formation of the two duplexes sufficiently binds polynucleotide 152, 952′ to the substrate that the polynucleotide may be amplified in a manner such as described with reference to FIGS. 10A-10E.

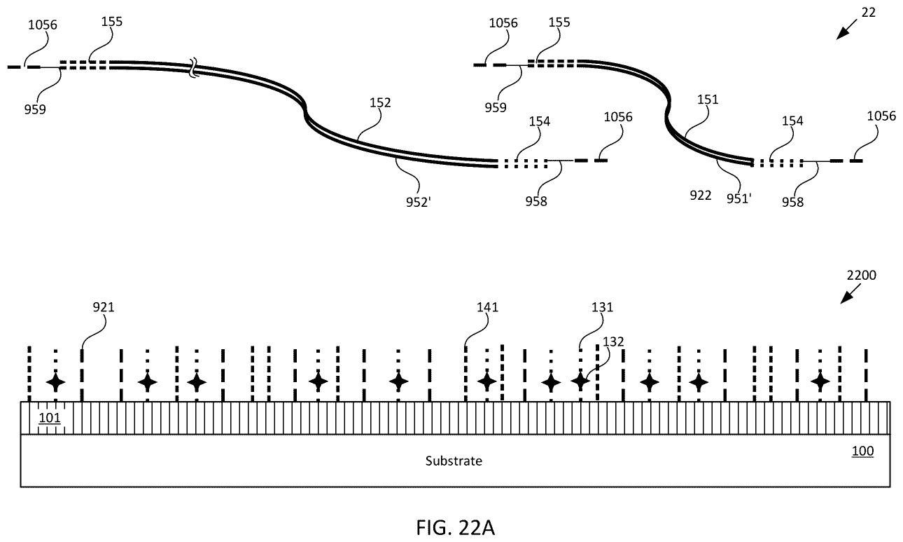

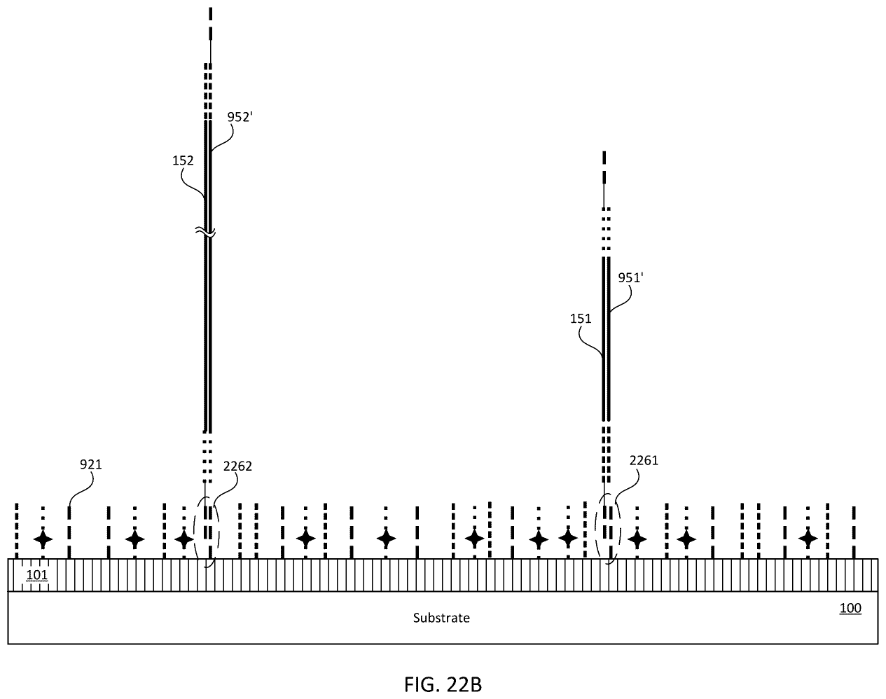

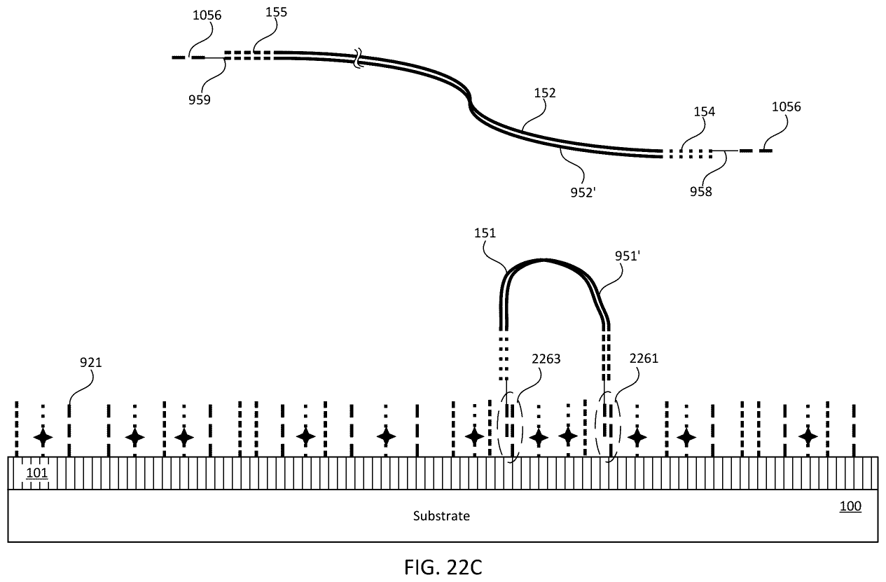

Although some examples provided herein may be used to selectively capture and amplify polynucleotides of certain lengths, it should be appreciated that such size selectivity is optional. As such, the examples suitably may be modified for use in capturing and amplifying polynucleotides of any length, and may result in substantially monoclonal clusters. For example, FIGS. 22A-22C schematically illustrate additional example structures and operations for capturing and amplifying polynucleotides using seeding adapters and seeding primers. As illustrated in FIG. 22A, structure 2200 may be contacted with double-stranded polynucleotides, illustrative a first double-stranded polynucleotide 151, 951′ and with a double-stranded polynucleotide 152, 952′. Similarly as described with reference to FIG. 10A, structure 2200 may include substrate 100, first and second amplification primers 131, 141 coupled to the substrate, and first and second seeding primers coupled to the substrate. However, while the first and second seeding primers 921, 922 in FIG. 10A may have different sequences than one another and are located in different regions of the substrate than one another and in different regions than primers 131, 141, in the example shown in FIG. 22A the first and second seeding primers 921, 921 may have the same sequences as one another and may be in a location of the substrate that includes a mixture of seeding primers 921 and amplification primers 131, 141. Seeding primers 921 illustrated in FIGS. 22A-22C may be configured similarly as described with reference to FIGS. 9A-9F.

Additionally, similarly as described with reference to FIG. 10A, polynucleotides 151, 951′ and 152, 952′ each may include first and second double-stranded amplification adapters 154, 155 and first and second single-stranded seeding adapters. However, while the seeding adapters 1056, 1057 have different sequences than one another in the example of FIG. 10A, the polynucleotides in the example of FIG. 22A may have first and second seeding adapters 1056 with the same sequence as one another, e.g., may include two copies of seeding adapter 1056, each coupled to a respective strand of the double-stranded polynucleotide. Seeding adapters 1056 illustrated in FIGS. 22A-22C may be configured similarly as described with reference to FIGS. 10A-10E.

As illustrated in FIG. 22B, the first single-stranded seeding adapter 1056 may be hybridized to the first seeding primer 921 to form a first duplex. The duplexes formed between seeding primers 921 and seeding adapters 1056 may be configured similarly as described with reference to FIGS. 10A-10E. For example, a single-stranded seeding adapter 1056 of polynucleotide 151, 951′ may hybridize to seeding primer 921 to form duplex 2261, and a single-stranded seeding adapter 1056 of polynucleotide 152, 952′ may hybridize to a different seeding primer 921 to form duplex 2262. As illustrated in FIG. 22C, while the first single-stranded seeding adapter is hybridized to the first seeding primer, the second single-stranded seeding adapter of that polynucleotide may hybridize to the second seeding primer to form a second duplex. For example, the other single-stranded seeding adapter 1056 of polynucleotide 151, 951′ may hybridize to another seeding primer 921 to form duplex 2263. In a manner similar to that described with reference to FIGS. 9A-9F, the duplexes 2261, 2263 together retain the double-stranded polynucleotide 151, 951′ at the substrate for sufficient time to amplify the double-stranded polynucleotide using the first and second double-stranded amplification adapters and the first and second amplification primers. Further details of strand invasion reactions that may be used to amplify double-stranded polynucleotide 151, 951′ are provided with reference to FIGS. 9A-9F and 10A-10E.

However, as noted elsewhere herein, the duplexes between seeding adapters 1056 and seeding primers 921 intentionally may be made relatively unstable, such that a single duplex alone does not retain the double-stranded polynucleotide at the substrate for sufficient time to amplify the double-stranded polynucleotide. This instability can discourage multiple different double-stranded polynucleotides from seeding a particular region of substrate (that is, can encourage monoclonality). In the non-limiting example shown in FIG. 22C, the instability of duplex 2262 between adapter 1056 of polynucleotide 152, 952′ and primer 921 causes that duplex to dissociate before the other adapter 1056 of that polynucleotide can hybridize to a different primer, and as such the polynucleotide 152, 952′ returns to solution instead of being amplified. As such, it is expected that the region of the substrate illustrated in FIG. 22C will be substantially monoclonal following amplification of polynucleotide 151, 951′ and result in a structure similar to that illustrated in FIG. 9E, except that the amplicons may be distributed throughout the illustrated region of the substrate. Excision moiety 132 may be used to selective remove amplicons which are oriented in a first direction, in a manner similar to that illustrated in FIG. 9F.

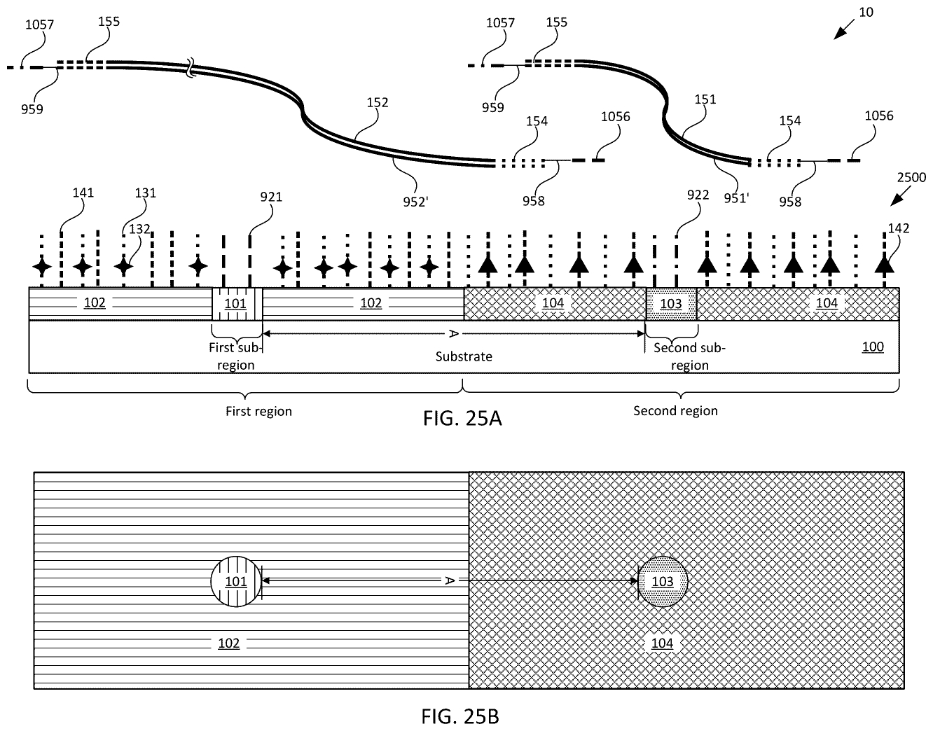

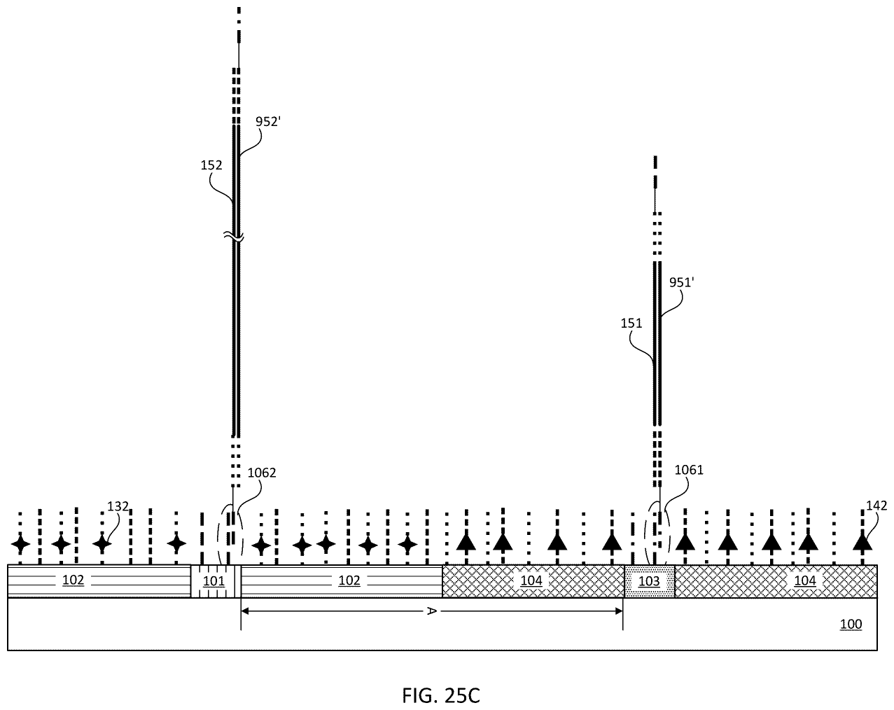

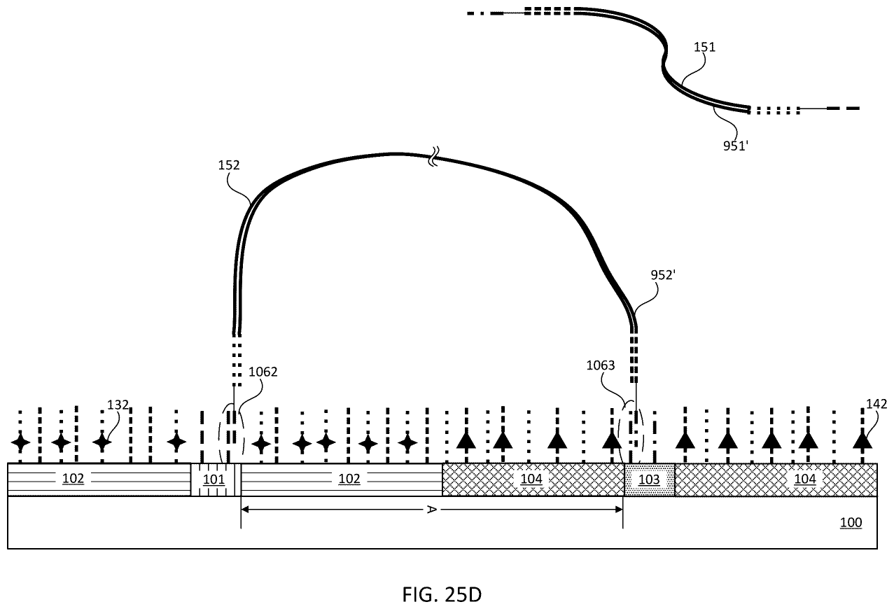

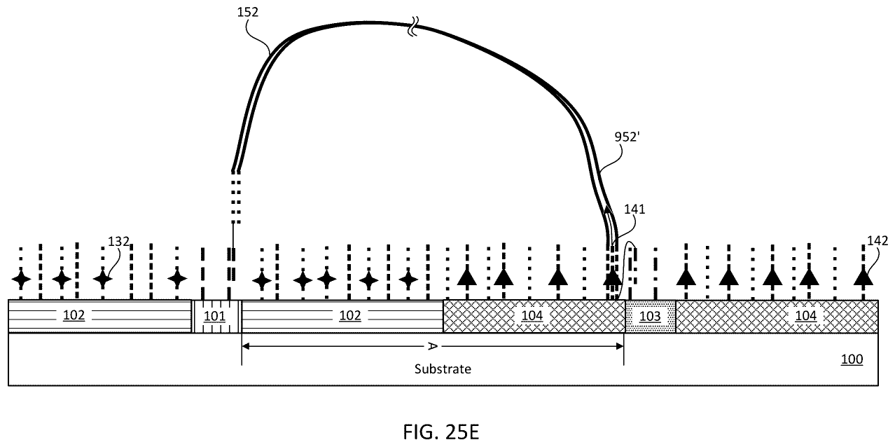

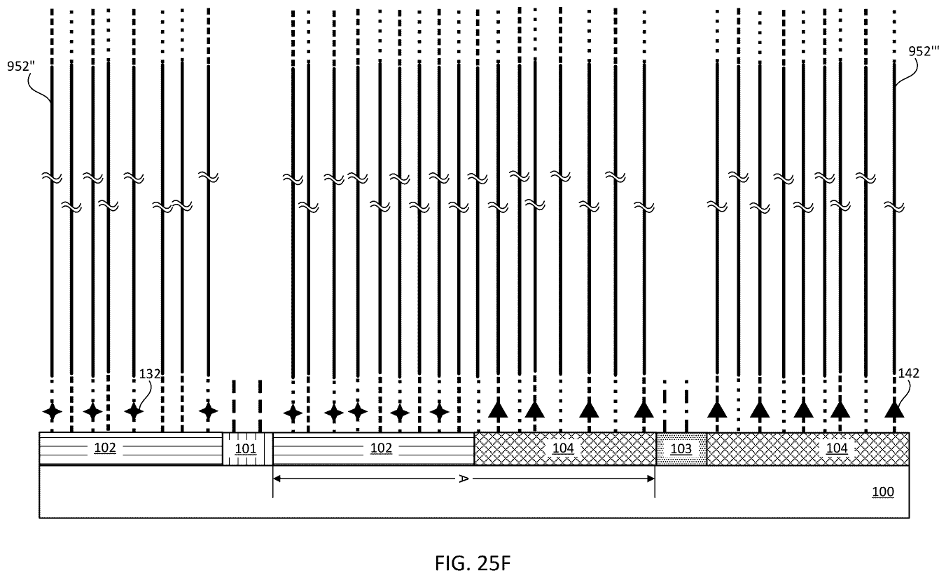

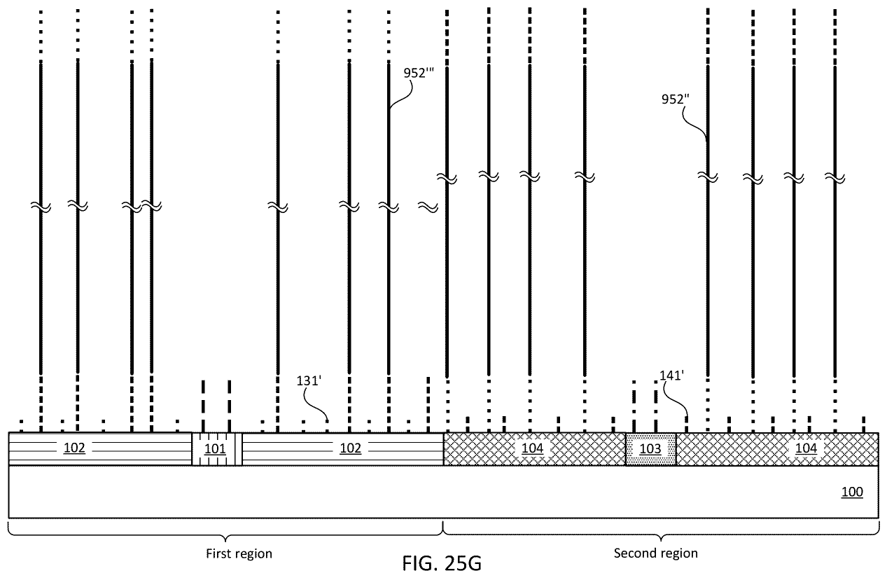

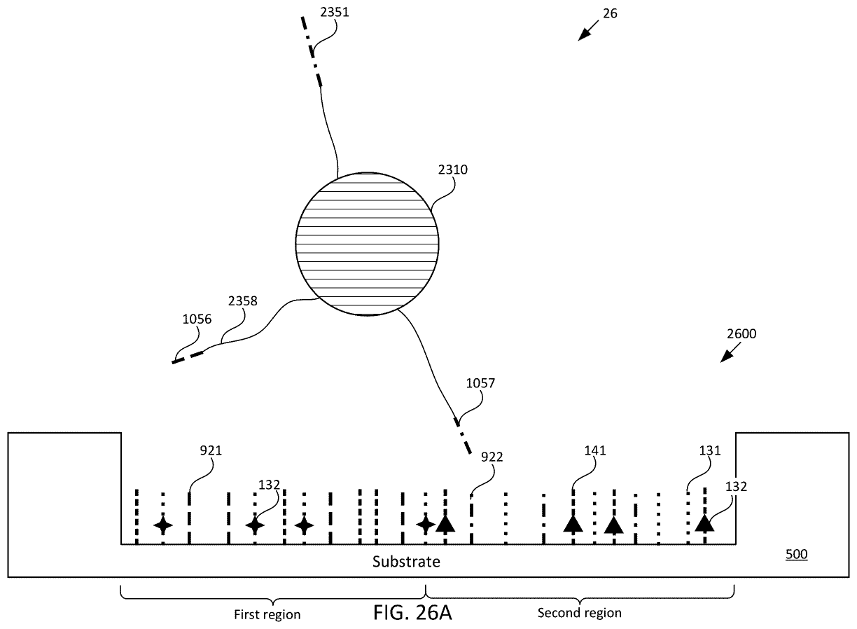

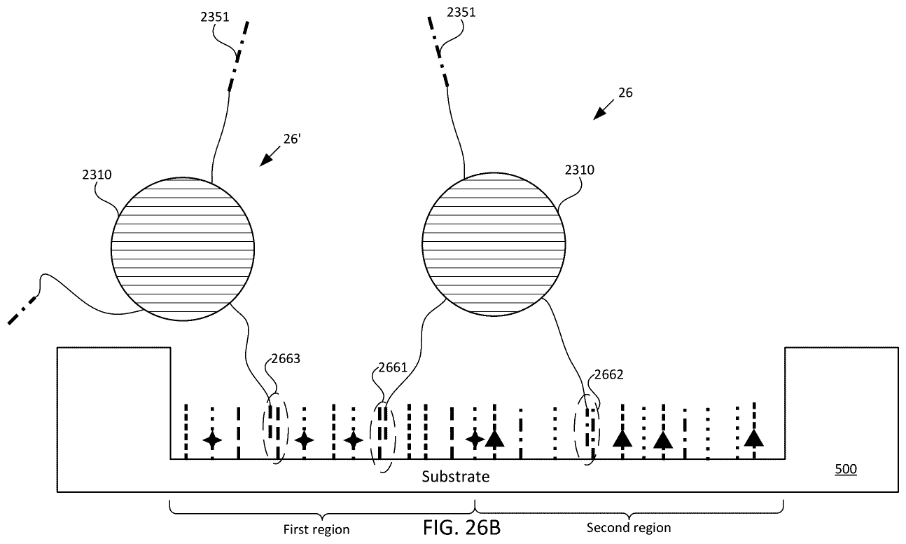

Various examples provided herein may be used to generate structures suitable for simultaneous paired-end reads on amplicons of the same double-stranded polynucleotide in two different regions of the substrate. For example, FIGS. 25A-25G schematically illustrate additional example structures and operations for capturing and amplifying polynucleotides using seeding adapters and seeding primers. As illustrated in FIG. 25A, structure 2500 may be contacted with double-stranded polynucleotides, illustrative a first double-stranded polynucleotide 151, 951′ and with a double-stranded polynucleotide 152, 952′. Similarly as described with reference to FIG. 10A, structure 2500 may include substrate 100, first and second amplification primers 131, 141 coupled to the substrate, and first and second seeding primers coupled to the substrate. Similarly as described with reference to FIG. 10A, the first and second seeding primers 921, 922 may have different sequences than one another and are located in different regions (e.g., sub-regions) of the substrate than one another and in different regions than primers 131, 141, e.g., as shown in simplified plan view in FIG. 25B. Additionally, in the example shown in FIG. 25A, a first mixture of amplification primers 131, 141 may be located in a first region of the substrate (e.g., the region containing the sub-region with seeding primers 921), and a second mixture of amplification primers 131, 141 may be located in a second region of the substrate (e.g., the region containing the sub-region with seeding primers 922). In the first region of the substrate, amplification primers 131 include a first excision moiety 132, while the second amplification primers in the second region of the substrate include a second excision moiety 142 that is orthogonal to the first excision moiety, e.g., that may be cleaved in a different manner than may be the first excision moiety. Different hydrogels may be patterned so as to dispose different primers, or different combinations of primers, in different regions of the substrate 100. For example, hydrogel 102 to which the first mixture of primers 131, 141 may be coupled may be patterned in the first region of the substrate, and hydrogel 101 to which primers 921 are coupled may be patterned in the first sub-region of the substrate. Hydrogel 104 to which the second mixture of primers 131, 141 may be coupled may be patterned in the second region of the substrate, and hydrogel 103 to which primers 922 are coupled may be patterned in the second sub-region of the substrate.

Additionally, similarly as described with reference to FIG. 10A, polynucleotides 151, 951′ and 152, 952′ each may include first and second double-stranded amplification adapters 154, 155 and first and second single-stranded seeding adapters 1056, 1057 which have different sequences than one another. As illustrated in FIG. 25C, the first single-stranded seeding adapter 1056 may be hybridized to the first seeding primer 921 to form a first duplex. The duplexes formed between seeding primers 921 and seeding adapters 1056 may be configured similarly as described with reference to FIGS. 10A-10E. For example, a single-stranded seeding adapter 1056 of polynucleotide 151, 951′ may hybridize to seeding primer 922 to form duplex 1061, and a single-stranded seeding adapter 1056 of polynucleotide 152, 952′ may hybridize to seeding primer 921 to form duplex 1062.

As illustrated in FIG. 25D, while the first single-stranded seeding adapter is hybridized to the first seeding primer at duplex 1062, the second single-stranded seeding adapter of that polynucleotide may hybridize to the second seeding primer to form a second duplex 1063 similarly as described with reference to FIG. 10D. In a manner similar to that described with reference to FIG. 10D, the duplexes 1062, 1063 together retain the double-stranded polynucleotide 152, 952′ at the substrate for sufficient time to amplify the double-stranded polynucleotide using the first and second double-stranded amplification adapters and the first and second amplification primers, while a single duplex alone does not retain the double-stranded polynucleotide at the substrate for sufficient time to amplify the double-stranded polynucleotide. In the non-limiting example shown in FIG. 25D, the instability of duplex 1061 between adapter 1056 of polynucleotide 151, 951′ and primer 922 causes that duplex to dissociate before the other adapter 1056 of that polynucleotide can hybridize to a different primer, and as such the polynucleotide 151, 951′ returns to solution instead of being amplified.

Furthermore, the gap A between primers 921 and 922 provides selectivity for sufficiently long polynucleotides, in a manner similar to that described with reference to FIGS. 9A-9F. Note also that the first and second duplexes 1062, 1063 retain the double-stranded polynucleotide 152, 952′ at approximately an interface between the first and second regions of the substrate. As such, it is expected that the first and second regions of the substrate illustrated in FIG. 25D will be substantially monoclonal following amplification of polynucleotide 152, 952′. Further details of strand invasion reactions that may be used to amplify double-stranded polynucleotide 152, 952′ are provided with reference to FIGS. 9A-9F and 10A-10E.

Briefly, as illustrated in FIG. 25E, double-stranded polynucleotide 152, 952′ may be amplified at one end or at both ends using a strand invasion process such as described with reference to FIGS. 9A-9F (amplification at one end, using primer 141, being illustrated in FIG. 25E). Similarly as described with reference to FIGS. 10A-10E, various elements illustrated in FIGS. 25A-25G may be flexible, and so can move to permit certain interactions between various elements. For example, although adapter 1057 may be hybridized to primer 922, the linker 959 between that adapter 1057 and adapter 155 may be flexible, and/or the hydrogel to which primer 141 is coupled may be flexible, so as to permit primer 141 to strand invade adapter 155 for amplification. As illustrated in FIG. 25E, recombinase-promoted extension of the primer 141 to which double-stranded polynucleotide 152, 952′ hybridizes forms amplicon 952″ which is covalently coupled to hydrogel 104 in the second region of the substrate. Amplicon 952″ repeatedly may be further amplified using strand invasion. For example, it may be seen that the composition of FIG. 25F includes a plurality of amplicons 952″ and 952′″ that are formed using a mixture of capture primers 131 and 141. Note that amplicons 952″ have a first orientation relative to the substrate (e.g., are coupled to the substrate via primers 131) and amplicons 952′″ have a second orientation relative to the substrate (e.g., are coupled to the substrate via primers 141). If amplification operations are repeated until the first and second substrate regions are substantially full, both adapters of the resulting amplicons may not necessarily be hybridized to corresponding capture primers or orthogonal capture primers, and as such the amplicons may extend linearly away from the substrate as illustrated in FIG. 25F.

As noted further above, capture primers 131 in the first region of the substrate (e.g., capture primers 131 which are coupled to hydrogel 102 and are not coupled to hydrogel 104) may include excision moieties 132, while capture primers 141 in the second region of the substrate (e.g., capture primers 141 coupled to hydrogel 104 and are not coupled to hydrogel 102) may include excision moieties 142. Excision moieties 132 may be used to selectively remove amplicons 952″ in the first orientation in the first region of the substrate, and excision moieties may be used to selectively remove amplicons 952′″ in the second orientation in the second region of the substrate. For example, as illustrated in FIG. 25G, following use of excision moieties 132 and 142, the first region of the substrate may include amplicons 952′″ in the second orientation, and residual stumps 131′ where amplicons 952″ previously had been located; and the second region of the substrate may include amplicons 952″ in the first orientation, and residual stumps 141′ where amplicons 952′″ previously had been located. Amplicons 952″ in the first region, and amplicons 952′″ in the second region, may be sequenced simultaneously as one another using sequencing-by-synthesis (SBS) to enhance robustness of the sequencing data obtained.

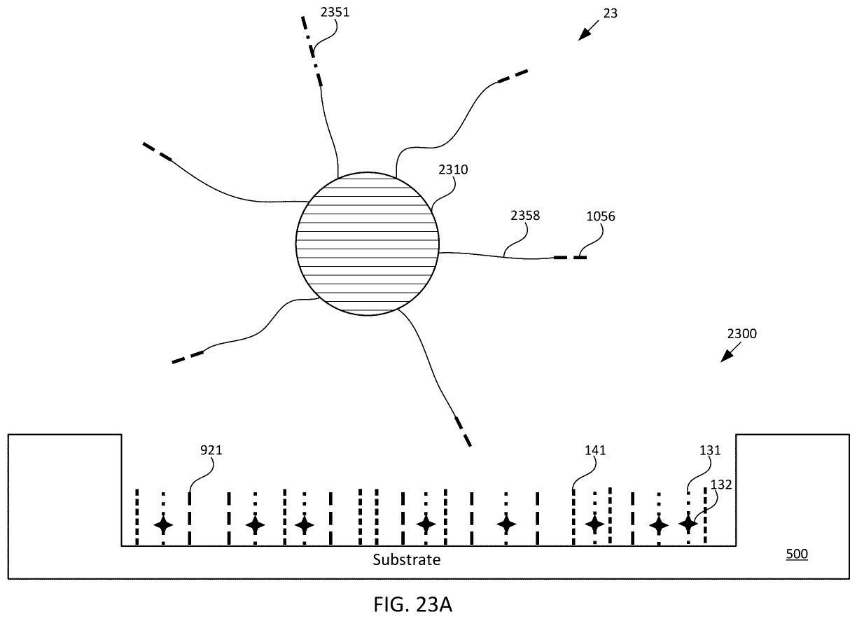

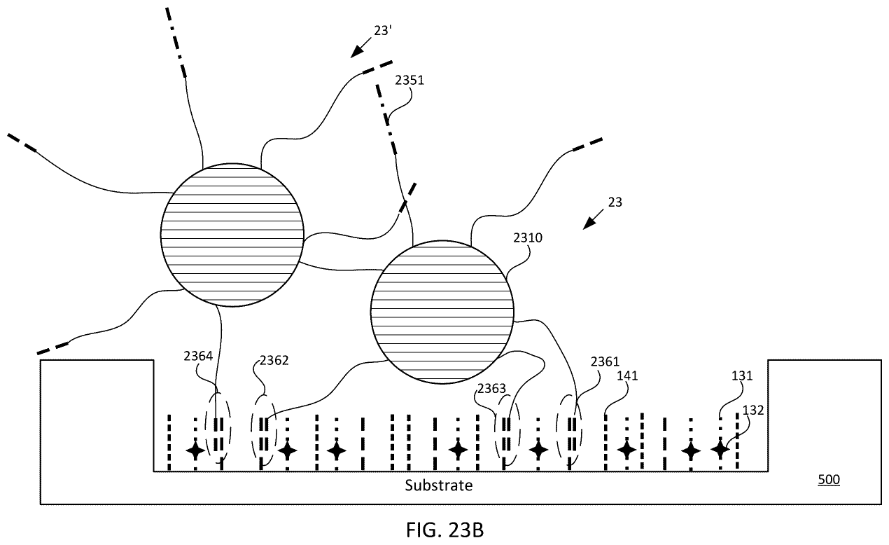

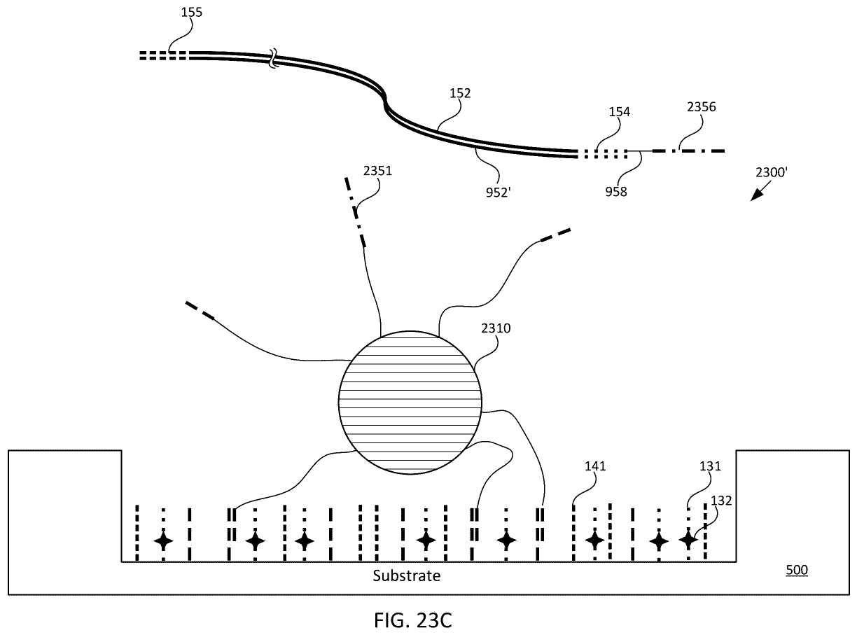

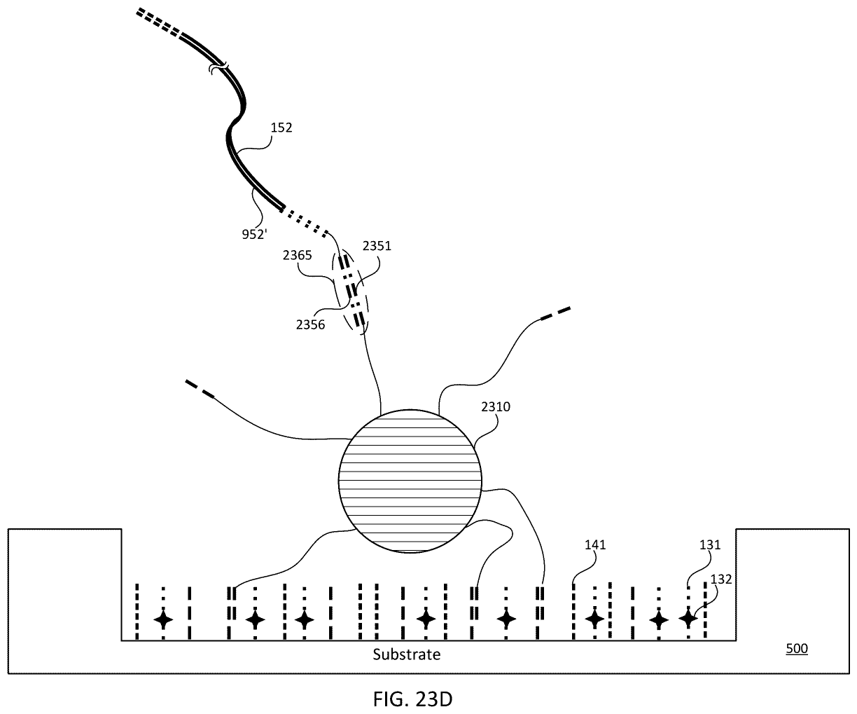

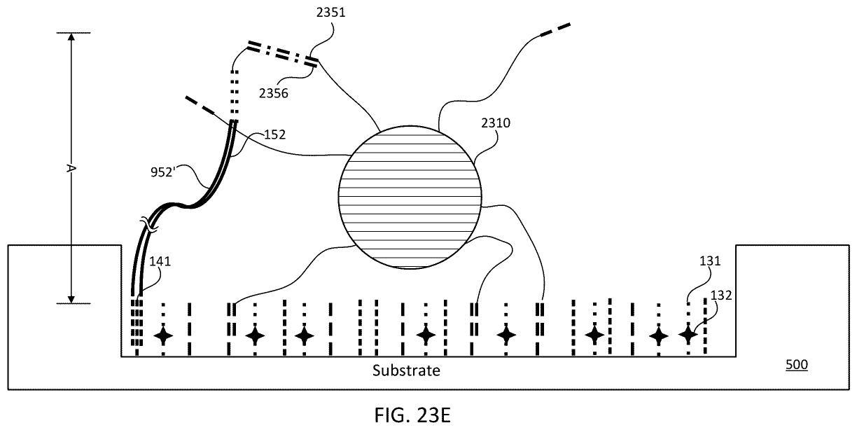

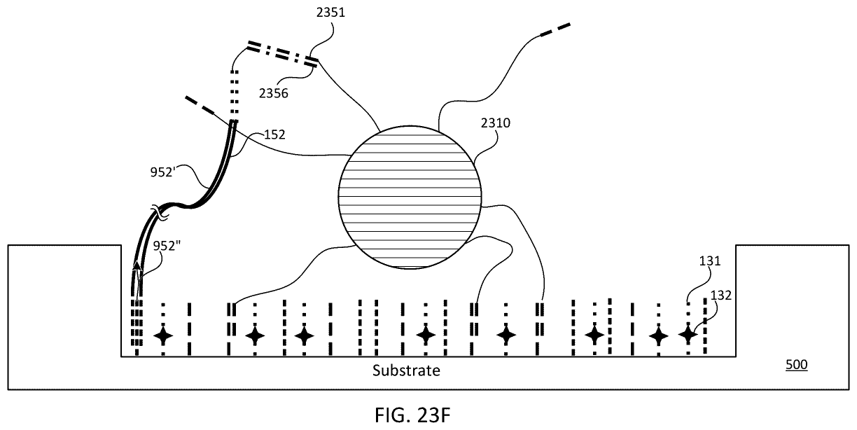

Still other structures and methods may be used to provide multiple unstable duplexes which encourage monoclonality in a manner such as described with reference to FIGS. 10A-10E, 22A-22C, and 25A-25G. For example, FIGS. 23A-23F schematically illustrate additional example structures and operations for capturing and amplifying polynucleotides using seeding adapters and seeding primers. Referring first to FIG. 23A, structure 2300 is contacted with a scaffold 23. Structure 2300 may include substrate 500 which may be configured similarly as described with reference to FIGS. 5A-5B (e.g., optionally may include a well), first and second amplification primers 131, 141 coupled to the substrate, and first and second seeding primers coupled to the substrate. In a manner similar to that described with reference to FIGS. 22A-22D and as illustrated in FIG. 23A, first and second seeding primers 921, 921 may have the same sequence as one another, and a different sequence than those of amplification primers 131, 141, and may be configured similarly as described with reference to FIGS. 9A-9F. The first and second seeding primers 921, 921 and the first and second amplification primers 131, 141 may be located in a region of the substrate in which a mixture of first and second seeding primers and first and second amplification primers is located.

Scaffold 23 may include core 2310, first and second single-stranded seeding adapters 1056, 1056 coupled to core 2310, and a third seeding primer 2351 coupled to core 2310. The seeding adapters 1056 and seeding primer 2351 may be coupled to scaffold 23 via respective linkers 2358. In some examples, linkers 2358 may be part of core 2310. For example, core 2310 may include at least one of a nanoparticle, DNA dendrimer, a polymer dendrimer, a peptide dendrimer, a polymer with bottlebrush structure, a single strand DNA with bottlebrush structure, a single strand DNA produced by rolling circle amplification (RCA), or a polypeptide scaffold. Further details of such examples are provided further below. Alternatively, linkers 2358 may be coupled to core 2310 via reaction between a first moiety coupled to the core and a second moiety coupled to the linker. Nonlimiting examples of linkers include those described with reference to linker 958. Illustratively, linkers 2358 may include poly(ethylene glycol) (PEG), alkyl chains, or combinations thereof. The linkers 2358, whether part of core 2310 or coupled to core 2310, may be coupled to seeding adapters 1056 and seeding primer 2351 in a manner such as disclosed further below. In a manner similar to that described with reference to FIGS. 22A-22D and as illustrated in FIG. 23A, seeding adapters 1056, 1056 may have the same sequence as one another and may be configured similarly as described with reference to FIGS. 10A-10E. Seeding primer 2351 may have a sequence that is different than, and orthogonal to, those of seeding primers 921 and amplification primers 131, 141 so that the seeding primer 2351 substantially may not interact with any of such primers and thus may not form a duplex that otherwise may bind scaffold 23 to the substrate. Illustratively, seeding primer 2351 may have the sequence PX.

As illustrated in FIG. 23B, the first single-stranded seeding adapter 1056 may be hybridized to the first seeding primer 921 to form a first duplex 2361. While the first single-stranded seeding adapter 1056 is hybridized to the first seeding primer, the second single-stranded seeding adapter 1056 may be hybridized to the second seeding primer 921 to form a second duplex 2362. Optionally, one or more additional seeding adapters 1056 may hybridize to respective seeding primers 921 to form one or more additional duplexes, e.g., duplex 2363. In a manner similar to that described with reference to FIGS. 10A-10E and 22A-22C, the first and second duplexes 2361, 2362 (and optionally any additional duplexes with adapters 1056 of scaffold 23) together retain the scaffold 23 at the substrate 500 for sufficient time to amplify a double-stranded polynucleotide in a manner such as will be described below (e.g., using first and second double-stranded amplification adapters 154, 155 and the first and second amplification primers 131, 141). In comparison, the first duplex 2361 alone, or the second duplex 2362 alone (or any other duplex alone), does not retain the scaffold 23 at substrate 500 for sufficient time to amplify a double-stranded polynucleotide. For example, based on formation of a single duplex, scaffold 23 may dissociate from substrate 500 and go back into solution. The number of hybridization events needed to stably retain the double-stranded polynucleotide at the substrate suitably may be adjusted. For example, while FIG. 23B may suggest that one hybridization event is insufficient but two hybridization events are sufficient, the number and characteristics of seeding adapters 1056 may be tuned such that even a relatively low number of binding events (e.g., 5 or 10 binding events) are insufficient and a relatively high number of binding events (e.g., 50+ hybridization events) are necessary for effective binding.

Additionally, as illustrated in FIG. 23B, the presence of a scaffold 23 at a given location on the substrate may sterically inhibit other such scaffolds 23′ from being able to become stably coupled to that location. For example, scaffold 23 may occupy a relatively large portion of the well of substrate 500, e.g., through hybridization of multiple of its seeding adapters 1056 to corresponding seeding primers 921. As such, a relatively small number of seeding primers 921 may be available for hybridization to a seeding adapter 1056 that is coupled to another scaffold 23′. Scaffold 23, or the well within substrate 500, can be sized such that even if a first seeding adapter 1056 of scaffold 23′ is able to hybridize to a first seeding primer 921, scaffold 23 may block a second seeding adapter 1056 of that scaffold 23′ from hybridizing to a second seeding primer 921. As such, scaffold 23′ may dissociate from substrate 500 and go back into solution. As such, scaffold 23′ may dissociate from substrate 500 and go back into solution. Illustratively, in some examples scaffold 23 may have a volume which is at least about 50% of the volume of the well within substrate 500. In other examples, the volume of scaffold 23 may be less than about 50% the volume of the well, and the dimensions of the scaffold are such that adapters 1056 may hybridize to a sufficient number of the surface primers to reduce the likelihood of a second scaffold sufficiently hybridizing in the same well. For example, scaffold 23 may be configured as a bottlebrush like structure that is anisotropic in shape and may wrap around parts of the well to access primers across the whole well. Additionally, or alternatively, in some examples the lengths of linkers 2358 may be selected to provide greater access of adapters 1056 on the scaffold to primers 921 at the surface to exclude the adapters of other scaffolds from hybridizing to those primers. Additionally, or alternatively, having a deficiency of surface primers 921 relative to adaptor primers 1056 may be beneficial for this size exclusion effect. However, in the even if there are more primers 921 than adapters 1056, the reduced binding affinity may assist in inhibiting individual or low numbers of binding events from lingering on the surface.

Referring now to FIG. 23C, scaffold 23 may be contacted with a double-stranded polynucleotide including first and second double-stranded amplification adapters and a third single-stranded seeding adapter. For example, scaffold 23 may be contacted with a fluid that includes a plurality of such double-stranded polynucleotides, of which polynucleotide 152, 952′ illustrated in FIG. 23C is one example. In a manner similar to that described with reference to FIGS. 9A-9F, double-stranded polynucleotide 152, 952′ may include amplification adapters 154 and 155. Polynucleotide 152, 952′ also may include a seeding adapter 2356 which is coupled to adapter 154 via linker 958. As illustrated in FIG. 23D, the single-stranded seeding adapter 2356 may hybridize to the third seeding primer 2351 to form a third duplex 2365. Such hybridization may be performed while the first single-stranded seeding adapter 1056 is hybridized to the first seeding primer 921 and while the second single-stranded seeding adapter 1056 is hybridized to the second seeding primer 921. In other examples, the third single-stranded seeding adapter is hybridized to the third seeding primer before the first single-stranded seeding adapter is hybridized to the first seeding primer and before the second single-stranded seeding adapter is hybridized to the second seeding primer, e.g., before scaffold 23 is introduced to the substrate. Duplex 2365 may have a melting temperature similar to those described above for the duplex between amplification adapter 154 and amplification primer 131, and/or the duplex between amplification adapter 155 and amplification primer 141. Illustratively, seeding adapter 2356 may have the sequence PX′ (also referred to herein as cPX). In such an example, duplex 2365 may be configured similarly as duplexes 961, 962, or 963 described with reference to FIG. 9B.

The double-stranded polynucleotide 152, 952′ then may be amplified in a similar manner as described with reference to FIGS. 9A-9F. For example, the polynucleotide may bridge gap A such that adapter 155 of double-stranded polynucleotide 152, 952′ hybridizes with one of primers 141 to form a duplex using “strand invasion” and may be promoted using a recombinase (not specifically illustrated in FIG. 23E). FIG. 23F illustrates the composition of FIG. 23E during recombinase-promoted extension of the primer 141 to which double-stranded polynucleotide 152, 952′ hybridizes to form amplicon 952″ which is covalently coupled to substrate 500 (e.g., to a hydrogel disposed on the substrate, not specifically illustrated) and then may be further amplified in a manner such as described with reference to FIGS. 9A-9F and 10A-10E. In this example, capture primers 131 may include excision moieties 132 which may be used to remove amplicons that are oriented in a selected direction, so that the remaining amplicons are oriented substantially in the same direction as one another in a manner similar to that illustrated in, and described with reference to, FIG. 9F.

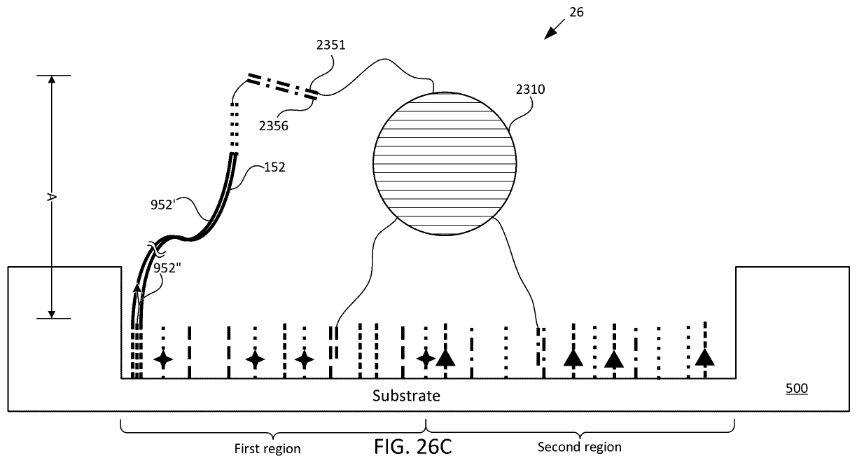

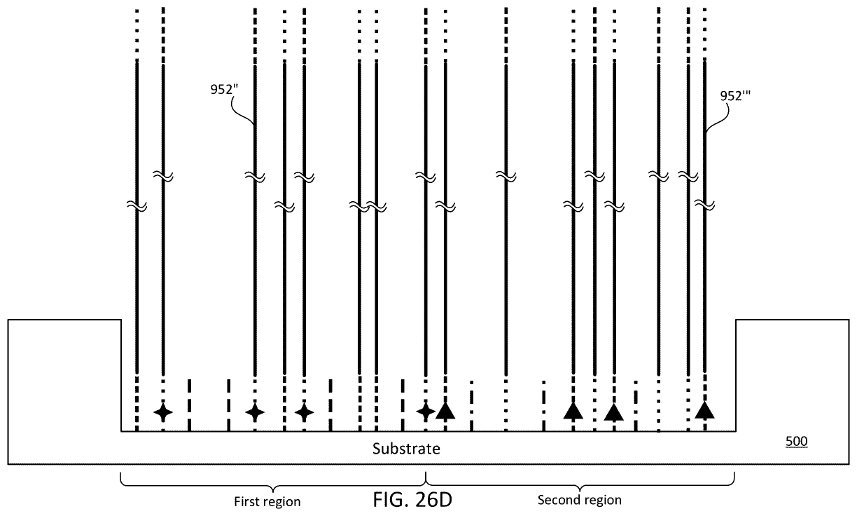

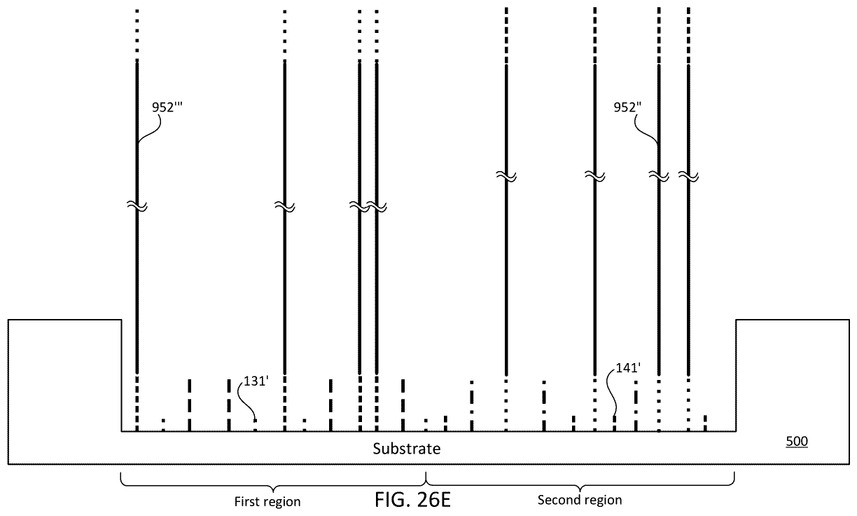

The examples described with reference to FIGS. 23A-23F may be modified to even further enhance monoclonality, and provide for simultaneous paired-end reads similar to those described with reference to FIGS. 25A-25G. For example, FIGS. 26A-26E schematically illustrate additional example structures and operations for capturing and amplifying polynucleotides using seeding adapters and seeding primers. Referring first to FIG. 26A, structure 2600 is contacted with a scaffold 26. Structure 2600 may include substrate 500 which may be configured similarly as described with reference to FIGS. 5A-5B (e.g., optionally may include a well), first and second amplification primers 131, 141 coupled to the substrate, and first and second seeding primers 921, 922 coupled to the substrate. In a manner similar such as illustrated in FIG. 26A, the first and second seeding primers 921, 922 may have different sequences than one another and are located in different regions of the substrate than one another. For example, first seeding primers 921 may be located in a first region of substrate 500 and not in a second region of substrate 500, while second seeding primers 922 may be located in the second region of the substrate and not in the first region of the substrate.

Additionally, in the example shown in FIG. 26A, a first mixture of amplification primers 131, 141 may be located in the first region of the substrate (e.g., the region with seeding primers 921), and a second mixture of amplification primers 131, 141 may be located in the second region of the substrate (e.g., the region with seeding primers 922). In the first region of the substrate, amplification primers 131 include a first excision moiety 132, while the second amplification primers in the second region of the substrate include a second excision moiety 142 that is orthogonal to the first excision moiety, e.g., that may be cleaved in a different manner than may be the first excision moiety. Different hydrogels may be patterned so as to dispose different primers, or different combinations of primers, in different regions of the substrate 100. For example, a first hydrogel (not specifically illustrated) to which seeding primers 921 and the first mixture of primers 131, 141 may be coupled may be patterned in the first region of the substrate, and a second hydrogel (not specifically illustrated) to which seeding primers 922 and the second mixture of primers 131, 141 may be coupled may be patterned in the second region of the substrate.

Scaffold 26 illustrated in FIG. 26A may include core 2310, first and second single-stranded seeding adapters 1056, 1057 coupled to core 2310, and a third seeding primer 2351 coupled to core 2310. The seeding adapters 1056, 1057 and seeding primer 2351 may be coupled to scaffold 26 via respective linkers 2358. Scaffold 26 illustrated in FIG. 26A may be configured similarly as scaffold 23 described with reference to FIGS. 23A-23F, except that the example scaffold 26 shown in FIG. 26A may include first and second seeding adapters that are different than one another, instead of multiple seeding adapters that are the same as one another. Seeding adapters 1056, 1057 may be configured similarly as described with reference to FIGS. 10A-10E. Seeding primer 2351 may have a different sequence than those of seeding primers 921 and amplification primers 131, 141, and illustratively may have the sequence PX.

As illustrated in FIG. 26B, the first single-stranded seeding adapter 1056 may be hybridized to the first seeding primer 921 to form a first duplex 2661. While the first single-stranded seeding adapter 1056 is hybridized to the first seeding primer, the second single-stranded seeding adapter 1057 may be hybridized to the second seeding primer 922 to form a second duplex 2662. In a manner similar to that described with reference to FIGS. 23A-23F, the first and second duplexes 2661, 2662 together retain the scaffold 26 at the substrate 500 for sufficient time to amplify a double-stranded polynucleotide in a manner such as will be described below (e.g., using first and second double-stranded amplification adapters 154, 155 and the first and second amplification primers 131, 141). In comparison, the first duplex 2661 alone, or the second duplex 2662 alone, does not retain the scaffold 26 at substrate 500 for sufficient time to amplify a double-stranded polynucleotide. For example, based on formation of a single duplex, scaffold 26 may dissociate from substrate 500 and go back into solution. The number of hybridization events needed to stably retain the double-stranded polynucleotide at the substrate suitably may be adjusted. For example, while FIG. 26B may suggest that one hybridization event is insufficient but two hybridization events are sufficient, the number and characteristics of seeding adapters 1056, 1057 may be tuned such that even a relatively low number of binding events (e.g., 5 or 10 binding events) are insufficient and a relatively high number of binding events (e.g., 50+ hybridization events) are necessary for effective binding.

Additionally, as illustrated in FIG. 26B, the presence of a scaffold 26 at a given location on the substrate may sterically inhibit other such scaffolds 26′ from being able to become stably coupled to that location. For example, in a manner similar to that described with reference to FIG. 23B, scaffold 26 may occupy a relatively large portion of the well of substrate 500, e.g., through hybridization of first seeding adapter 1056 to corresponding seeding primer 921 and through hybridization of second seeding adapter 1057 to corresponding seeding primer 922. Additionally, because seeding primers 921 are located in one region of substrate 500, and seeding primers 922 are located in a different region of substrate 500, scaffold 26 may be retained approximately at the interface between the first and second regions of the substrate via duplex 2661 (in the first region) and duplex 2662 (in the second region). As such, scaffold 26 may inhibit other such scaffolds 26′ from being able to access that interface as well, and thus may inhibit other such scaffolds 26′ from being able to form a first duplex with a first seeding primer 921 and to form a second duplex with a second seeding primer 922. As such, that other scaffold 26′ may be able to primarily access only seeding primers 921 for hybridization to a seeding adapter 1056 coupled to scaffold 26′ to form a single duplex 2663 as is illustrated in FIG. 26B; or similarly may be able to primarily access only seeding primers 922 for hybridization to a seeding adapter 1057 coupled to scaffold 26′ to form a single duplex (not specifically illustrated in FIG. 26B). Accordingly, even if a first seeding adapter 1056 of scaffold 26′ is able to hybridize to a first seeding primer 921, scaffold 26 may block a second seeding adapter 1057 of that scaffold 26′ from hybridizing to a second seeding primer 922. As such, scaffold 26′ may dissociate from substrate 500 and go back into solution.

Scaffold 26 may be contacted with a double-stranded polynucleotide 152, 952′ including first and second double-stranded amplification adapters 154, 155 and a third single-stranded seeding adapter 2351, e.g., in a manner similar to that described with reference to FIG. 23C. Such hybridization may be performed while the first single-stranded seeding adapter 1056 is hybridized to the first seeding primer 921 and while the second single-stranded seeding adapter 1057 is hybridized to the second seeding primer 922. In other examples, the third single-stranded seeding adapter is hybridized to the third seeding primer before the first single-stranded seeding adapter is hybridized to the first seeding primer and before the second single-stranded seeding adapter is hybridized to the second seeding primer, e.g., before introducing scaffold 26 to the substrate. The double-stranded polynucleotide 152, 952′ then may be amplified in a similar manner as described with reference to FIGS. 9A-9F. For example, as illustrated in FIG. 26C, the polynucleotide may bridge gap A such that adapter 155 of double-stranded polynucleotide 152, 952′ hybridizes with one of primers 141 to form a duplex using “strand invasion” and may be promoted using a recombinase (not specifically illustrated in FIG. 26C). The amplicon then may be further amplified in a manner such as described with reference to FIGS. 9A-9F, 10A-10E, and 25A-25G. For example, it may be seen that the composition of FIG. 26D includes a plurality of amplicons 952″ and 952″ that are formed using a mixture of capture primers 131 and 141. Similarly as in FIG. 25F, amplicons 952″ illustrated in FIG. 26D have a first orientation relative to the substrate (e.g., are coupled to the substrate via primers 131) and amplicons 952′″ have a second orientation relative to the substrate (e.g., are coupled to the substrate via primers 141). If amplification operations are repeated until the first and second substrate regions are substantially full, both adapters of the resulting amplicons may not necessarily be hybridized to corresponding capture primers or orthogonal capture primers, and as such the amplicons may extend linearly away from the substrate as illustrated in FIG. 26D.

As noted further above, capture primers 131 in the first region of the substrate (e.g., capture primers 131 which are coupled to hydrogel 102 and are not coupled to hydrogel 104) may include excision moieties 132, while capture primers 141 in the second region of the substrate (e.g., capture primers 141 coupled to hydrogel 104 and are not coupled to hydrogel 102) may include excision moieties 142. Excision moieties 132 may be used to selectively remove amplicons 952″ in the first orientation in the first region of the substrate, and excision moieties may be used to selectively remove amplicons 952′″ in the second orientation in the second region of the substrate. For example, as illustrated in FIG. 26E, following use of excision moieties 132 and 142, the first region of the substrate may include amplicons 952′″ in the second orientation, and residual stumps 131′ where amplicons 952″ previously had been located; and the second region of the substrate may include amplicons 952″ in the first orientation, and residual stumps 141′ where amplicons 952′″ previously had been located. Amplicons 952″ in the second region, and amplicons 952′″ in the first region, may be sequenced simultaneously as one another using sequencing-by-synthesis (SBS) to enhance robustness of the sequencing data obtained.

Further details of scaffolds 23 and 26 now will be provided. In some examples, scaffold 23 or scaffold 26 includes, consists essentially of, or is: a nanoparticle, a dendrimer, a polymer with bottlebrush structure, a single strand DNA scaffold, or a polypeptide scaffold, or a combination thereof. Nanoparticles including a scaffold, a site for bonding a template DNA to the scaffold, and a plurality of sites for bonding oligonucleotides to the scaffold, have been described in U.S. Publication No. 2021/0187469 A1, the entire contents of which are incorporated by reference herein. In some examples, scaffold 23 or scaffold 26 may include one or a plurality of scaffold DNA molecules, such as a DNA dendrimer. In other examples, scaffold 23 or scaffold 26 may include single-stranded DNA. In still other examples, scaffold 23 or scaffold may include one or more scaffold polypeptides.

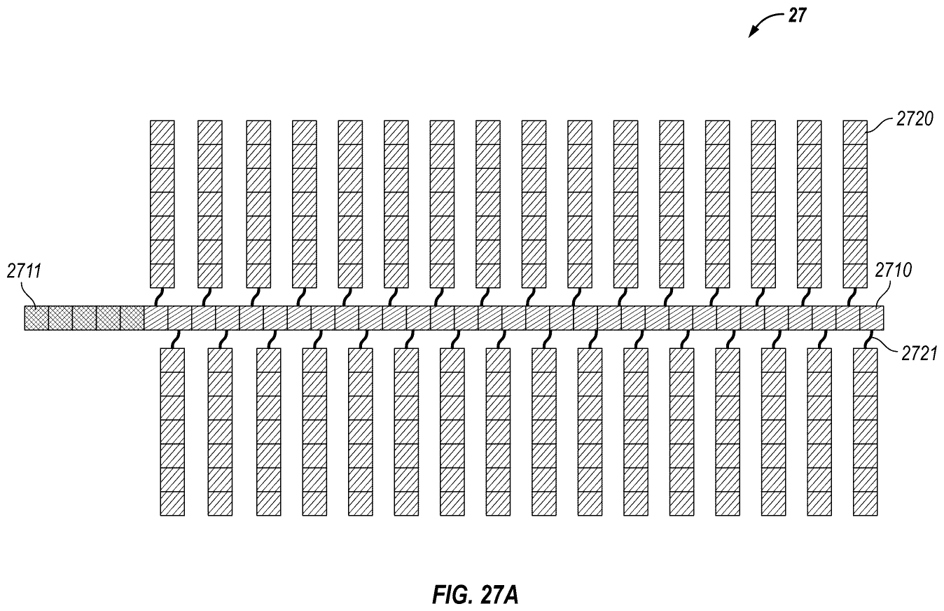

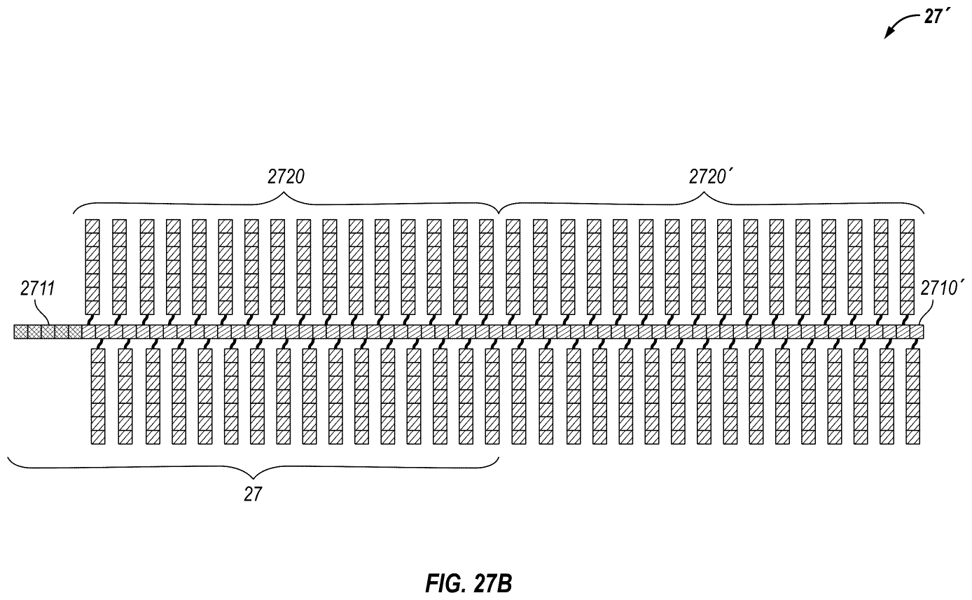

FIG. 27A illustrates a non-limiting example of a polynucleotide bottlebrush structure 27 that may be used in examples such as described with reference to FIGS. 23A-23F. Structure 27 illustrated in FIG. 27A includes central backbone 2710, corresponding to core 2310, that includes seeding primer 2711 corresponding to seeding primer 2351 described with reference to FIGS. 23A-23F and 26A-26E. A plurality of seeding adapters 2720, corresponding to seeding adapters 1056, are coupled along the length of central backbone 2710, e.g., via respective linkers 2721. In one non-limiting example, central backbone 2710 may be formed using seeding primer 2711 as a primer, and extending the primer using functionalized nucleotides. For example, central backbone 2710 may include a ssDNA scaffold synthesized by use of a template-independent polymerase (e.g., terminal deoxynucleotidyl transferase, or TdT). TdT incorporates deoxynucleotides at the 3-prime-hydroxyl terminus of a single-stranded DNA strand, without requiring or copying a template.

The functional groups of the nucleotides (e.g., azido groups) then may be coupled to seeding adapters 2720 which may be synthesized to include functional groups (e.g., alkyne groups such as DBCO) that react with those of the nucleotides in backbone 2710. FIG. 27B illustrates a non-limiting example of a polynucleotide bottlebrush structure 27′ that may be used in examples such as described with reference to FIGS. 26A-26E. Structure 27′ may be formed by first forming structure 27 in a manner such as described with reference to FIG. 27A, and optionally capping any remaining functional groups of the nucleotides in structure 27. Then the central backbone 2710 may be further extended using functionalized nucleotides. The functional groups of the nucleotides (e.g., azido groups) then may be coupled to seeding adapters 2720′, corresponding to seeding adapters 1057, e.g., which include functional groups that react with those of the available nucleotides in extended backbone 2710′. An alternative approach for preparing scaffold 26 is to use a mixture of modified dUTPs such that orthogonal reactions may be performed in tandem. For example, a vinyl-functionalized dUTP and an azido-functionalized dUTP may be used together to create backbone 2710, and orthogonal reactions may be used to couple seeding adapters 2720 and 2720′ thereto. Note that whichever way it is prepared, the length of backbone 2710, 2710′- and thus the number of seeding adapters coupled thereto—may be controlled through the duration of the extension reaction.

Another non-limiting example of a scaffold 23 or 26 may include a single strand DNA produced by rolling circle amplification (RCA) that includes a plurality of seeding adapters 1056.

In some examples, scaffold 23 or 26 may be synthesized so as to include, and may include once synthesized, more than one type of chemistry or structure for attachment. That is, scaffold 23 or 26 may be synthesized to include or be modified to include a moiety with a first chemistry for attaching seeding primer 2351, plus one or more additional moieties with a different chemistry for attaching seeding adapters 1056 and/or seeding adapters 1057.

In some examples, the core 2310 of scaffold 23 may be synthesized from one or more scaffold deoxyribonucleic acid (DNA) molecules. DNA molecules may be designed and structured as further disclosed herein so as to permit inclusion of different bonding sites (i.e., for seeding primer 2351 as well as seeding adapters 1056 and/or 1057) and also to provide size-exclusion properties for distancing double-stranded polynucleotides from each other once attached to substrate 500 in a manner such as described with reference to FIG. 23B or FIG. 26B. In some examples, the core 2310 of scaffold 23 or 26 may include a plurality of DNA molecules hybridized together so as to form a dendrimer. For example, adapters may be formed including a plurality of, such as three, strands of DNA, or oligodeoxyribonucleotide (oligo-DNA) molecules that can hybridize to each other by Watson-Crick base pairing so as to form a Y-shape, with one end of each hybridizing to one of the other two and the other end of each hybridizing to the other of the other two.

Such adapters may form a constitutional repeating until of a dendrimer. For example, each end of the Y-shaped adapter may have an overhang of DNA, where the end of one of the oligo-DNAs extends beyond the portion of which hybridizes to any other oligo-DNA. An adapter of one generation of such dendrimer may have an overhang on one end of the Y-shape, referred to here as the upstream end, that can hybridize with an overhang of an and of another Y adapter that constitutes a constitutional repeating unit of an immediately preceding generation of the dendrimer. And the other two ends of the adapter, referred to as the downstream ends, may each have an overhang that can hybridize with an overhang of an upstream end of a Y adapter that constitutes a constitutional repeating unit of an immediately following generation of the dendrimer. Thus, an adapter of one generation may attach to two adapters in the next generation, which may attach to four adapters of the following generation, which may attach to eight adapters of the following generation, and so on. Any one end of one of the terminal Y adapters, whether a downstream overhang of any generation, such as the last generation, not hybridized to an upstream overhang of another adapter, or the upstream overhang of the first generation, may include or be attached to the seeding primer 2351. In an example, the DNA-oligo including the upstream overhang of the first generation adapted may itself be an extension of a double-stranded polynucleotide to be sequenced, added thereto during sample preparation. Other ends or overhangs may include or be attached to seeding adapters 1056 and/or 1057.

In some examples, core 2310 of scaffold 23 or 26 may include one or more single-stranded DNA (ssDNA) molecules modified or structured so as to permit attachment thereto of seeding primer 2351 and seeding adapters 1056 and/or 1057. Various methods for producing an ssDNA-based core 2310 may be used. In an example, a double-stranded closed loop or plasmid may serve as a coding sequence for an ssDNA core molecule, in a rolling circle amplification process. Replication of a strand thereof by a strand-displacing DNA polymerase (e.g., Phi29) may produce an ssDNA molecule including concatemerized copies of the copied strand of the circular coding strand. Reaction conditions may be adopted so as to result in synthesis of an ssDNA core 2310 of a desired size. A 5-prime or 3-prime end may be further modified to include or be attached or attachable to seeding primer 2351. Seeding adapters 1056 and/or 1057 may include the other end of the ssDNA scaffold molecule, or modifications to or of individual nucleotides of the strand.

In another example, core 2310 may include a ssDNA scaffold synthesized by producing a plurality of single-stranded DNA molecules by any applicable method and ligating them together to form a single ssDNA molecule. For example, a polymerase may polymerize formation of a nascent strand of DNA by copying a linearized DNA coding strand, in a run-off polymerization reaction (i.e., where the polymerase ceases extending a nascent strand upon reaching a 5-prime end of a coding strand). A plurality of ssDNA products may be synthesized, then ligated end-to-end for formation of a ssDNA scaffold. In an example, ligation of one ssDNA product to another may be accomplished with the aid of a splint. For example, a short oligo-DNA may be designed whose 3-prime end is complementary of the 5-prime end of one ssDNA product and whose 5-prime end is complementary to the 3-prime end of another ssDNA product, such that hybridization of the DNA-oligo to the two ssDNA products brings the 5-prime end of one together with the 3-prime end of the other in a nicked, double-stranded structure where they meet hybridized to the DNA-oligo. A DNA ligase (e.g., T4) may then be used to enzymatically ligate the two ends together to form a single ssDNA molecule from the two. Additional reactions may be included with DNA-oligos for splint-aided ligation of one or both ends of the product of such first reaction to another ssDNA product, and so on, for construction of an ssDNA scaffold as may be desired.

In some examples, seeding primer 2351 and/or seeding adapters 1056 and/or seeding adapters 1057 are attached to scaffold 23 or 26 by noncovalent interaction. Illustratively, the noncovalent interaction may be avidin-biotin interaction. For example, seeding primer 2351 and/or seeding adapters 1056 and/or seeding adapters 1057 may include a biotin moiety that allows for non-covalent bonding with a respective streptavidin binding site located on the scaffold 23 or 26 (e.g., located on linker 2358). In other examples, seeding primer 2351 and/or seeding adapters 1056 and/or seeding adapters 1057 may include a streptavidin moiety that allows for non-covalent bonding with a respective biotin binding site located on the scaffold 23 or 26 (e.g., located on linker 2358).

In some examples, seeding primer 2351 and/or seeding adapters 1056 and/or seeding adapters 1057 are attached to scaffold 23 or 26 by covalent bonding. In some such examples, the covalent bonding is selected from the group consisting of amine-NHS ester bonding, amine-imidoester bonding, amine-pentofluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxoamine-aldehyde bonding, SpyTag-SpyCatcher bonding, Snap-tag-O⁶-benzylguanine bonding, CLIP-tag-O²-benzylcytosine bonding, and sortase-coupling bonding.

Illustratively, scaffold 23 or 26 (e.g., linker 2358) may include one or more amino bonding sites, carboxy bonding sites, thiol bonding sites, aldehyde bonding sites, azido bonding sites, hydroxy bonding sites, cycloalkene bonding sites (such as transcyclooctene bonding sites or norbornene bonding sites), cycloalkyne bonding sites (such as cyclooctyne bonding sites dibenzocyclooctyne (DBCO) bonding sites, or bicyclononyne bonding sites), oxoamine bonding sites, SpyTag bonding sites, Snap-tag bonding sites, CLIP-tag bonding sites, or proteins with N-terminus recognized by sortase, or combinations thereof. In some such examples, seeding primer 2351 and/or seeding adapters 1056 and/or seeding adapters 1057 respectively includes a functional moiety that allows for covalent bonding with the corresponding site of the scaffold. The functional moiety of the seeding primer 2351 and/or seeding adapters 1056 and/or seeding adapters 1057 independently may include or is selected from a NHS ester moiety, an aldehyde moiety, an imidoester moiety, a pentofluorophenyl ester moiety, a hydroxymethyl phosphine moiety, a carbodiimide moiety, a maleimide moiety, a haloacetyl moiety, a pyridyl disulfide moiety, a thiosulfonate moiety, a vinyl sulfone moiety, a hydrazine moiety, an alkoxyamine moiety, an isocyanate moiety, an alkyne moiety, a cycloalkyne moiety, a phosphine moiety, a tetrazine moiety, an azido moiety, a SpyCatcher moiety, an O⁶-Benzylguanine moiety, an O⁶-Benzylcytosine moiety, or a fragment that can be subject to sortase coupling. In other examples, the functional moiety on the scaffold 23 and the functional moiety of the seeding primer 2351 and/or seeding adapters 1056 may be reversed from the specific examples provided above.

A non-exclusive list of complementary binding partners is presented in Table 1:

TABLE 1

| | Example moiety on scaffold 23 | Example moiety on seeding adapter |
|---|---|---|
| | or 26 or seeding adapter 1056 or | 1056 or seeding adapter 1057 or |
| | seeding adapter 1057 or seeding | seeding primer 2351 or scaffold 23 or |
| Bonding site | primer 2351 | 26 |
| amine-NHS | amine group, —NH₂ | N-Hydroxysuccinimide ester 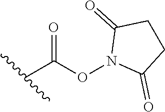 [SMILES: *C(=O)ON1C(=O)CCC1=O] |
| amine- imidoester | amine group, —NH₂ | imidoester 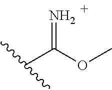 [SMILES: *C(=N)OC] |
| amine- pentofluorophenyl ester | amine group, —NH₂ | pentofluorophenyl ester, 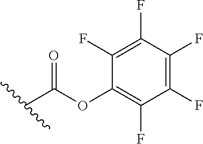 [SMILES: *C(=O)Oc1c(F)c(F)c(F)c(F)c1F] |
| amine- hydroxymethyl phosphine | amine group, —NH₂ | hydroxymethyl phosphine 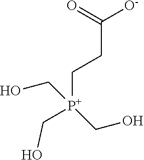 [SMILES: O=C([O-])CC[P+](CO)(CO)CO] |
| amine- carboxylic acid | amine group, —NH₂ | carboxylic acid group, —C(═O)OH (e.g., following activation of the carboxylic acid by a carbodiimide such as EDC (1- ethyl-3-(-3-dimethylaminopropyl) carbodiimide hydrochloride) or DCC (N′,N′-dicyclohexyl carbodiimide) to allow for formation of an amide bond of the activated carboxylic acid with an amine group) |
| thiol-maleimide | thiol, —SH | maleimide 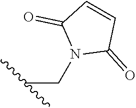 [SMILES: *CN1C(=O)C=CC1=O] |
| thiol-haloacetyl | thiol, —SH | haloacetyl (e.g., iodoacetyl or other haloacetyl) 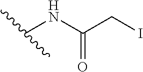 [SMILES: *NC(=O)CI] |
| thiol-pyridyl disulfide | thiol, —SH | pyridyl disulfide 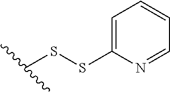 [SMILES: *SSc1ccccn1] |
| thiol- thiosulfonate | thiol, —SH | thiosulfonate 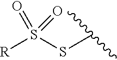 |
| thiol-vinyl sulfone | thiol, —SH | vinyl sulfone 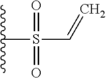 [SMILES: *S(=O)(=O)C=C] |
| aldehyde- hydrazide | aldehyde, —C(═O)H | hydrazide 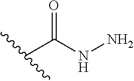 [SMILES: *C(=O)NN] |
| aldehyde- alkoxyamine | aldehyde, —C(═O)H | alkoxyamine 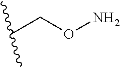 [SMILES: *CON] |
| hydroxy- isocyanate | hydroxyl, —OH | isocyanate 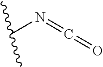 [SMILES: *N=C=O] |
| azide-alkyne | azide, —N₃ | alkyne 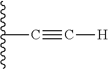 [SMILES: *C#C] |
| azide-phosphine | azide, —N₃ | phosphine, e.g.: 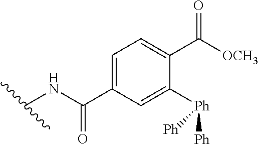 |
| azide- cyclooctyne | azide, —N₃ | cyclooctyne, e.g. dibenzocyclooctyne (DBCO) 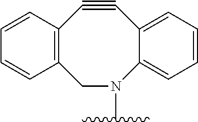 [SMILES: *N1Cc2ccccc2C#Cc2ccccc21] or BCN (bicyclo[6.1.0]nonyne) 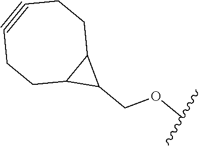 [SMILES: *OCC1C2CCC#CCCC21] |
| azide- norbornene | azine, —N₃ | norbornene 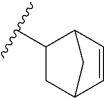 [SMILES: *C1CC2C=CC1C2] |
| transcyclooctene- tetrazine | transcyclooctene 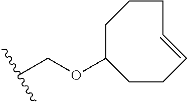 [SMILES: *COC1CC/C=C/CCC1] | tetrazine, e.g., benzyl-methyltetrazine 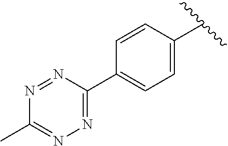 [SMILES: *c1ccc(-c2nnc(C)nn2)cc1] |
| norbornene- tetrazine | norbornene 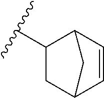 [SMILES: *C1CC2C=CC1C2] | tetrazine, e.g. benzyl-tetrazine 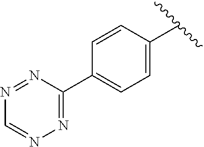 [SMILES: *c1ccc(-c2nncnn2)cc1] |
| oxime | aldehyde or ketone (e.g., amine | alkoxyamine |
| | cugroup or N-terminus of | |
| | polypeptide converted to an | |
| | aldehyde or ketone by pyroxidal | |
| | phosphate) | |
| SpyTag- | SpyTag: amino acid sequence | SpyCatcher amino acid sequence: |
| SpyCatcher | AHIVMVDAYKPTK (SEQ ID | MKGSSHHHHHHVDIPTTENLYFQ |
| | NO: 13) | GAMVDTLSGLSSEQGQSGDMTIEE |
| | | DSATHIKFSKRDEDGKELAGATME |
| | | LRDSSGKTISTWISDGQVKDFYLY |
| | | PGKYTFVETAAPDGYEVATAITFT |
| | | VNEQGQVTVNGKATK(SEQ ID |
| | | NO: 14) |
| SNAP-tag-O⁶ Benzylguanine | SNAP-tag (O-6-methylguanine- DNA methyltransferase) | O⁶-Benzylguanine 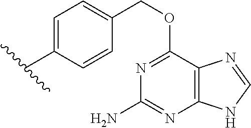 [SMILES: *c1ccc(COc2nc(N)nc3[nH]cnc23)cc1] |
| CLIP-tag-O²- benzylcytosine | CLIP-tag (modified O-6- methylguanine-DNA methyltransferase) | O²-benzylcytosine 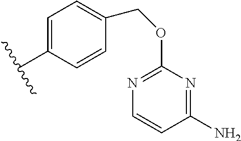 [SMILES: *c1ccc(COc2nccc(N)n2)cc1] |
| Sortase-coupling | -Leu-Pro-X-Thr-Gly | —Gly₍₃₋₅₎ |

Although FIGS. 10A-10E, 12A-12B, 22A-22C, 23A-23G, 25A-25G, and 26A-26E illustrate examples in which the seeding primers are relatively short so as to reduce the melting temperature of respective duplexes between seeding primers and seeding adapters, it will be appreciated that any suitable characteristic(s) of the seeding primers and/or seeding adapters may be selected so as to provide the resulting duplex with a suitable melting temperature. FIGS. 24A-24C schematically illustrate alternative duplexes that may be formed between seeding adapters and seeding primers in examples such as described with reference to FIGS. 10A-10E, 12A-12B, 22A-22C, 23A-23G, 25A-25G, and 26A-26E. In the nonlimiting example illustrated in FIG. 24A, seeding primer 921 and seeding adapter 1056 are approximately the same length as one another, and the melting temperature of their duplex is controlled via the respective compositions of seeding primer 921 and seeding adapter 1056. For example, the sequences of primer 921 and adapter 1056 may be selected to include a number of bases having a proportion of G-C pairings that yields a desired melting temperature. In the nonlimiting example illustrated in FIG. 24B, seeding primer 921 is relatively short compared to seeding adapter 1056, and the melting temperature of their duplex is controlled via the number and composition of the bases in the region where seeding primer 921 and seeding adapter 1056 overlap and hybridize to one another. In the nonlimiting example illustrated in FIG. 24C, seeding primer 921 is relatively long compared to seeding adapter 1056, and the melting temperature of their duplex is controlled via the number and composition of the bases in the region where seeding primer 921 and seeding adapter 1056 overlap and hybridize to one another. Examples such as described with reference to FIGS. 24A-24C apply equally to any duplexes formed between any seeding adapters and any seeding primers, e.g., such as described with reference to FIGS. 10A-10E, 12A-12B, 22A-22C, 23A-23G, 25A-25G, and 26A-26E.

From the foregoing disclosure, it will be appreciated that primers and adapters having any suitable sequences may be used. In one nonlimiting example, capture primers 131 are P5 amplification primers, and the capture primers 141 are P7 amplification primers. P5 amplification primers, which are commercially available from Illumina, Inc. (San Diego, CA) have the sequence 5′-AATGATACGGCGACCACCGA-3′ (SEQ ID NO: 1). P7 amplification primers, which also are commercially available from Illumina, Inc., have the sequence 5′-CAAGCAGAAGACGGCATACGA-3′ (SEQ ID NO: 2). Adapters 154 may be full-length complementary P5 adapters (cP5) having the sequence 5′-TCGGTGGTCGCCGTATCATT-3′ (SEQ ID NO: 3), and are commercially available from Illumina, Inc. Adapters 155 may be full-length complementary P7 adapters (cP7) having the sequence 5′-TCGTATGCCGTCTTCTGCTTG-3′ (SEQ ID NO: 4), and are commercially available from Illumina, Inc. Seeding primers 921 may have any suitable sequence which is orthogonal to the sequences of capture primers 131, 141, and, if used, 922. In some examples, seeding primers 921 or seeding primer 2351 may be PX primers having the sequence AGGAGGAGGAGGAGGAGGAGGAGG (SEQ ID NO: 5). Seeding primers 922 may be PY primers having the sequence 5′-GAA GAA GAA GAA GAA GAA GAA GAA GAA GAA-3′ (SEQ ID NO: 6). Seeding adapters 956 or 2356 may be cPX (also referred to as PX′) primers having the sequence CCTCCTCCTCCTCCTCCTCCTCCT (SEQ ID NO: 7). Seeding adapters 1056 may have a subset of the sequence of cPX, e.g., may have any of the sequences listed in Table 2 other than the full length PX sequence. Seeding adapters 1057 may have the sequence 5′-TTC TTC TTC TTC TTC TTC TTC TTC TTC TTC-3′ (SEQ ID NO: 8).

It will be appreciated that various examples herein may be used with operations consistent with “bridge amplification” or “surface-bound polymerase chain reaction” and/or with other amplification modalities. One such amplification modality is “exclusion amplification,” or ExAmp. Exclusion amplification methods may allow for the amplification of a single target polynucleotide per substrate region and the production of a substantially monoclonal population of amplicons in a substrate region. For example, the rate of amplification of the first captured target polynucleotide within a substrate region may be more rapid relative to much slower rates of transport and capture of target polynucleotides at the substrate region. As such, the first target polynucleotide captured in a substrate region may be amplified rapidly and fill the entire substrate region, thus inhibiting the capture of additional target polynucleotide in the same substrate region. Alternatively, if a second target polynucleotide attaches to same substrate region after the first polynucleotide, the relatively rapid amplification of the first polynucleotide may fill enough of the substrate region to result in a signal that is sufficiently strong to perform sequencing by synthesis (e.g., the substrate region may be at least functionally monoclonal). The use of exclusion amplification may also result in super-Poisson distributions of monoclonal substrate regions; that is, the fraction of substrate regions in an array that are functionally monoclonal may exceed the fraction predicted by the Poisson distribution.

Increasing super-Poisson distributions of useful clusters is useful because more functionally monoclonal substrate regions may result in higher quality signal, and thus improved SBS; however, the seeding of target polynucleotides into substrate regions may follow a spatial Poisson distribution, where the trade-off for increasing the number of occupied substrate regions is increasing the number of polyclonal substrate regions. One method of obtaining higher super-Poisson distributions is to have seeding occur quickly, followed by a delay among the seeded target polynucleotide. The delay, termed “kinetic delay” because it is thought to arise through the biochemical reaction kinetics, gives one seeded target polynucleotide an earlier start over the other seeded targets. Exclusion amplification works by using recombinase to facilitate the invasion of primers (e.g., primers attached to a substrate region) into double-stranded DNA (e.g., a target polynucleotide) when the recombinase mediates a sequence match. The present compositions and methods may be adapted for use with recombinase to facilitate the invasion of the present capture primers and orthogonal capture primers into the present target polynucleotides when the recombinase mediates a sequence match. Indeed, the present compositions and methods may be adapted for use with any surface-based polynucleotide amplification methods such as thermal PCR, chemically denatured PCR, and enzymatically mediated methods (which may also be referred to as recombinase polymerase amplification (RPA), strand invasion, or ExAmp).

Furthermore, although not specifically illustrated in all examples, it should be appreciated that a wall such as described with reference to FIG. 7 may be disposed between first and second regions of the substrate in examples in which the first and second regions of the substrate may include one or more different types of capture primers than one another. For example, a wall may be provided between the first and second regions described with reference to FIG. 9A-9F, 10A-10E, 25A-25G, or 26A-26E. Nonlimiting examples of processes for forming a wall are described elsewhere herein.

### Example Hydrogel Configurations

In examples such as described with reference to FIGS. 1A-11, 2-4, 5A-5B, 6, 7, 8A-8B, 9A-9F, 10A-10E, 11, 12A-12B, 22A-22C, 23A-23F, 24A-24C, 25A-25G, and 26A-26E, the capture primers (e.g., 131, 141, 921, and/or 922) optionally may be coupled to respective hydrogel(s) that are disposed on the substrate. The hydrogel itself may be covalently coupled to the substrate, or alternatively may be non-covalently coupled to the substrate. Some examples herein relate to structures having different configurations of hydrogels. Such structures optionally may be used in operations such as described elsewhere herein, but it should be appreciated that such structures may be used in other types of operations besides those expressly disclosed herein.

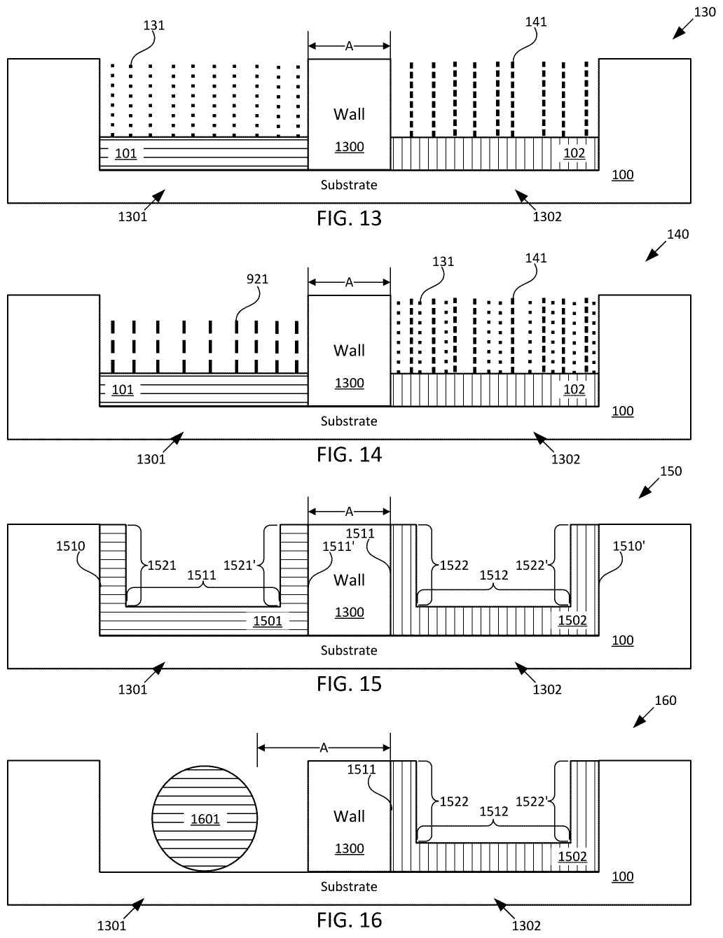

FIGS. 13-18 and 19A-19B schematically illustrate various alternative structures that include a material having a first recess and a second recess defined therein, the first recess being separated from the second recess by a wall; a first hydrogel disposed within the first recess; and a second hydrogel disposed within the second recess. The wall may separate the first hydrogel from the second hydrogel.

For example, referring now to FIG. 13, structure 130 includes substrate 100 having a first recess 1301 and a second recess 1302 defined therein, the first recess being separated from the second recess by wall 1300; a first hydrogel 101 disposed within the first recess; and a second hydrogel 102 disposed within the second recess. The wall 1300 may separate the first hydrogel 101 from the second hydrogel 102. In this example, the first and second recesses 1301, 1302 respectively may correspond to the first and second regions of the substrate described further above. Optionally, the first hydrogel 101 is coupled to a first set of capture primers, and the second hydrogel 102 is coupled to a second set of capture primers. In the nonlimiting examples illustrated in FIGS. 13 and 14, capture primers of the first set are of a different type than capture primers of the second set. In these and other figures herein, it will be appreciated that the length of the primers relative to the depth of the recesses may be greatly exaggerated for simplicity of illustration and discussion. In the example shown in FIG. 13, the first set of capture primers includes a first set of amplification primers, e.g., primers 131 described elsewhere herein. Additionally, in the example shown in FIG. 13, the second set of capture primers includes a second set of amplification primers, e.g., primers 141 described elsewhere herein. In the example structure 140 shown in FIG. 14, the first set of capture primers includes seeding primers, e.g., primers 921 or 922 described elsewhere herein. Additionally, in the example shown in FIG. 14, the second set of capture primers includes amplification primers, e.g., a mixture of primers 131 and 141.

Although FIGS. 13 and 14 may suggest that the hydrogels 101, 102 are substantially disposed on a single, planar surface of substrate, the hydrogels may be disposed on additional surfaces as well. For example, referring now to FIG. 15, structure 150 includes substrate 100 having a first recess 1301 and a second recess 1302 defined therein, the first recess being separated from the second recess by wall 1300; a first hydrogel 1501 disposed within the first recess; and a second hydrogel 1502 disposed within the second recess. Similarly as described with reference to FIG. 13, the wall 1300 may separate the first hydrogel 1501 from the second hydrogel 1502. In this example, the first and second recesses 1301, 1302 respectively may correspond to the first and second regions of the substrate described further above. Optionally, the first hydrogel 1501 is coupled to a first set of capture primers, and the second hydrogel 1502 is coupled to a second set of capture primers; such capture primers may be configured in a manner such as described with reference to FIG. 13 or 14, or elsewhere herein.

In the example illustrated in FIG. 15, portion 1511 of hydrogel 1501 is disposed on a first horizontal region of substrate 100, portion 1521 of hydrogel 1501 is disposed on at least partially vertical sidewall 1510 of substrate 100, and portion 1521′ of hydrogel 1501 is disposed on at least partially vertical sidewall 1511′ of wall 1300. Similarly, portion 1522 of hydrogel 1502 is disposed on a second horizontal region of substrate 100, portion 1522 of hydrogel 1502 is disposed on at least partially vertical sidewall 1510′ of substrate 100, and portion 1522′ of hydrogel 1502 is disposed on at least partially vertical sidewall 1511 of wall 1300. As such, recesses 1301 and 1302 may contain a significantly larger volume of their respective hydrogel than described with reference to FIGS. 13 and 14. In examples in which hydrogels 1501 and 1502 are coupled to primers in a manner such as described with reference to FIG. 13 or 14, used for cluster amplification in a manner such as described elsewhere herein or otherwise known in the art, and the clusters then used for sequencing (e.g., SBS), the larger volumes of hydrogels 1501 and 1502 may include greater numbers of primers and thus may provide an enhanced signal to noise ratio during sequencing as compared to hydrogels 101 and 102 illustrated in FIGS. 13 and 14.

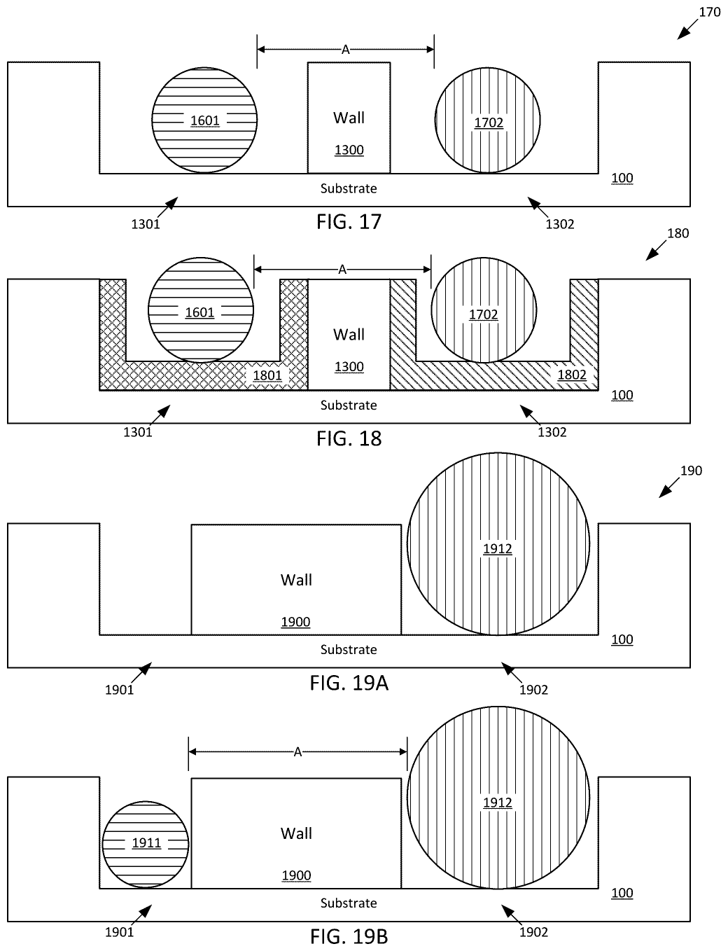

In examples such as shown in FIGS. 13, 14, and 15, wall 1300 defines the gap A between the first and second hydrogels. However, as noted elsewhere herein, the gap may be larger than wall 1300, and the wall may be within the gap. For example, one or both of the hydrogels may be patterned within recesses 1301 and 1302. Additionally, or alternatively, one or both of the hydrogels may be in the form of a particle which is inserted into a respective recess. FIGS. 16-18 and 19A-19B illustrate a nonlimiting example in which the first hydrogel is on or in a first particle disposed within the first recess. In the example structure 160 shown in FIG. 16, first hydrogel 1601 is on or in a particle disposed within recess 1301. For example, the particle may consist essentially of the first hydrogel 1601, optionally including one or more types of primers such as described elsewhere herein (e.g., primers 921, 922, 131, and/or 141). Second hydrogel 1502 is disposed within recess 1301 and may be configured similarly as described with reference to FIG. 15. In this example, gap A may correspond to the distance between sidewall 1511 and the edge of first hydrogel 1601 that is nearest to wall 1300. In the example structure 170 shown in FIG. 17, first hydrogel 1601 is on or in a particle disposed within recess 1301 and may be configured similarly as described with reference to FIG. 16. Second hydrogel 1702 is disposed within recess 1302 and may be configured similarly as hydrogel 1502 described with reference to FIG. 15. In this example, gap A may correspond to the distance between the edge of first hydrogel 1601 that is nearest to wall 1300 and the edge of second hydrogel 1702 that is nearest to wall 1300. Example structure 180 shown in FIG. 18 is configured similarly as structure 170, except that the first and second recesses 1301, 1302 respectively include additional materials 1801, 1802 on which the hydrogels 1601, 1702 respectively are disposed. Optionally, materials 1801, 1802 may include a hydrogel that is different in at least one respect from hydrogel 1601 or hydrogel 1702. For example, materials 1801, 1802 may lack primers. Materials 1801, 1802 may include the same type of material as one another, or may include different types of material than one another. For example, material 1801 may be configured to attract hydrogel 1601, and optionally to covalently or non-covalently couple thereto. Additionally, or alternatively, material 1802 may be configured to attract hydrogel 1702, and optionally to covalently or non-covalently couple thereto. For example, materials 1801 and 1802 may have orthogonal functionalities, e.g., so as to allow loading of hydrogels 1601 and 1702 simultaneously. Illustratively, materials 1801 and 1802 may include different moieties such as described in Table 1.

Although FIGS. 13-16 may suggest that recesses 1301, 1302 are approximately the same size as one another, it should be appreciated that the recesses may be different sizes than one another. For example, FIGS. 19A-19B illustrate an example structure 190 in which recess 1901 is smaller than recess 1902. For example, the size and/or location of wall 1900 may be modified so as to change the relative sizes of recesses 1901 and 1902 relative to recesses 1301 and 1302 illustrated in FIGS. 13-18. The differently sized recesses 1901, 1902 optionally may be used to facilitate selectively disposing different types of hydrogels, which are disposed on or in different sizes of particles, within such recesses. For example, as shown in FIG. 19A, second hydrogel 1912 may be on or in a second particle. Second hydrogel particle 1912 may be larger than recess 1901, and thus sterically hindered from becoming disposed within recess 1901. Second hydrogel particle 1912 may be smaller than recess 1902, and may become disposed within second recess 1902 in a manner such as illustrated in FIG. 19A. The resulting structure then may be contacted with first hydrogel 1911 which may be on or in a second particle that is smaller than hydrogel particle 1912. Although first hydrogel particle 1911 may be smaller than recess 1902, because hydrogel particle 1912 is already disposed within that recess the first hydrogel particle is sterically hindered from becoming disposed within recess 1902. First hydrogel particle 1911 may be smaller than recess 1902, and may become disposed within first recess 1901 in a manner such as illustrated in FIG. 19B. As such, the different particle sizes and different recess sizes may be used to control which type of hydrogel particle may be disposed in which recess of the substrate.

Although FIGS. 13-18 and 19A-19B illustrate various configurations including two types of hydrogel (which optionally may be coupled to respective sets of primer(s)), it should be appreciated that any of such configurations readily may be adapted to include additional type(s) of hydrogels. For example, such configurations readily may be modified so as to include one or more additional hydrogels disposed on the substrate, which optionally may be coupled to one or more additional sets of capture primers in a manner such as described with reference to FIGS. 10A-10E, 12A-12B, 25A-25G, and 26A-26E.

Forming Structures with Walls and/or Recesses

Some examples herein relate to forming structures that may be used in operations such as described in operations 1A-11, 2-4, 5A-5B, 6, 7, 8A-8B, 9A-9F, 10A-10E, 11, 12A-12B, 13-18, 19A-19B, 22A-22C, 23A-23F, 24A-24C, 25A-25G, and 26A-26E. While the structures formed may be used in such operations, it will be appreciated that the structures are not so limited, and may be used in other operations instead. Furthermore, it will be appreciated that the present structures may be formed in any suitable manner, and are not limited to being formed in the manner which will now be described.

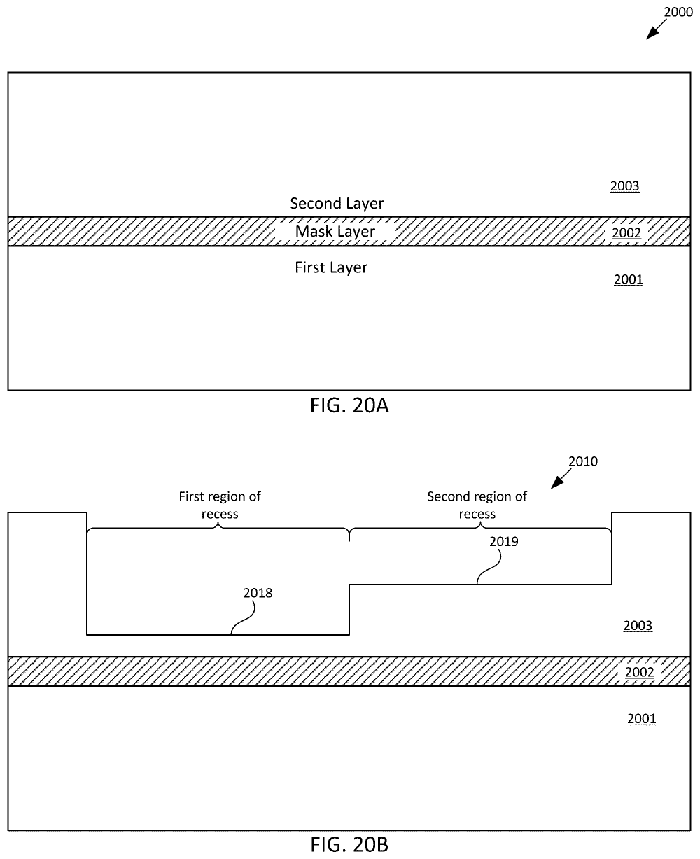

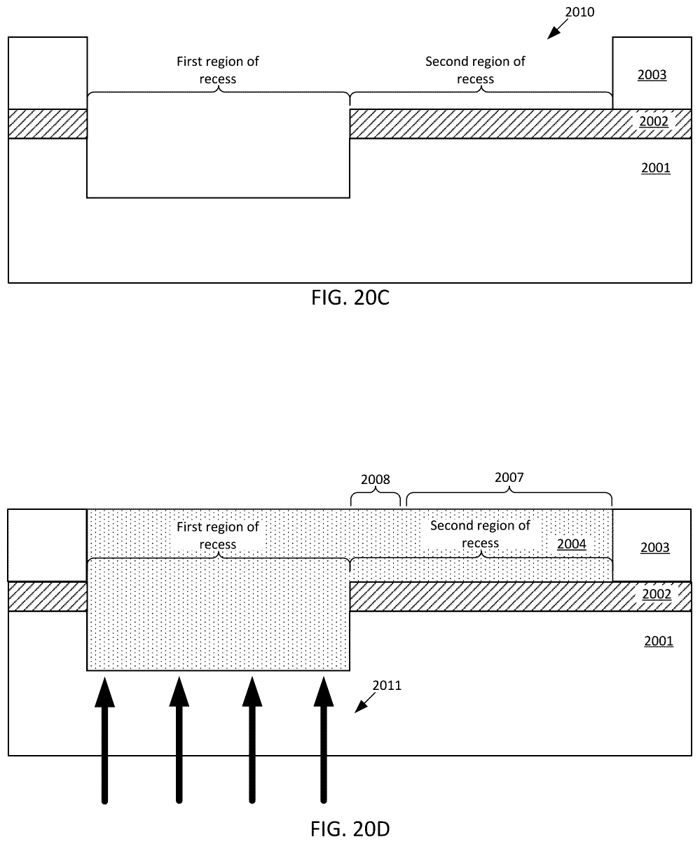

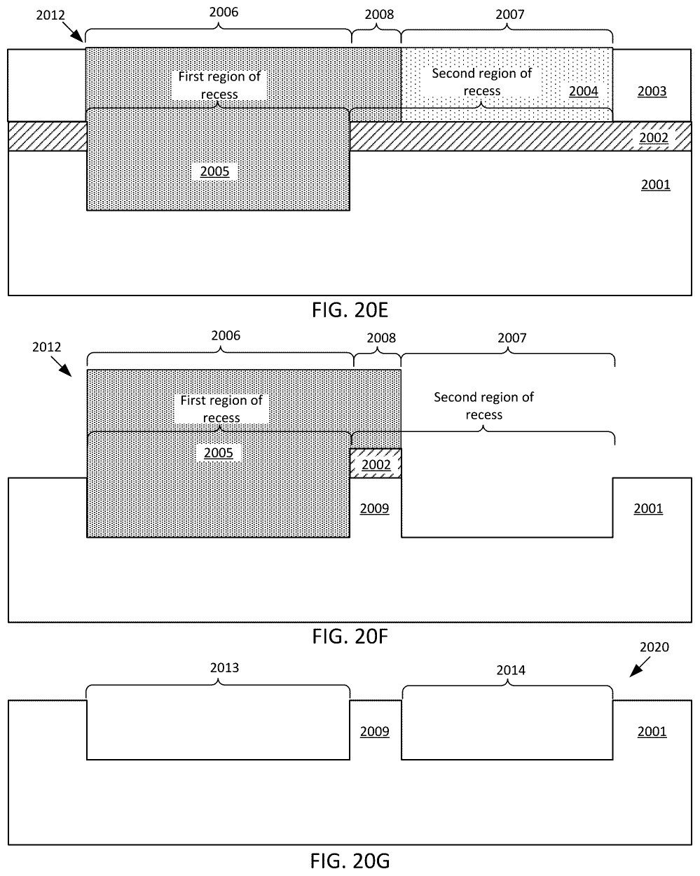

FIGS. 20A-20G schematically illustrate example operations for patterning, e.g., forming recesses in, a substrate 2000. As illustrated in FIG. 20A, substrate 2000 may include a first layer 2001, a mask layer 2002 disposed on the first layer, and second layer 2003 disposed on the mask layer 2002. As illustrated in FIG. 20B, a recess 2010 may be formed in the second layer 2003 of substrate 2002. Recess 2010 may be asymmetrical. For example, recess 2010 may include a first region and a second region. As illustrated in FIG. 20B, a lower surface 2018 of the first region may be deeper within the second layer than a lower surface 2019 of the second region. Illustratively, recess 2010 may be formed in the second layer 2003 of the substrate 2000 using nano-imprint lithography (NIL). For example, second layer 2003 may include, or may essentially consist of, a resin, into which the recess is impressed. The resin may include a lift-off resist, e.g., such as polymethylglutaride-based resists which are commercially available from Kayaku Advanced Materials, Inc. Optionally, the material of first layer 2001 (first material) also may include a resin. Alternatively, the material of first layer 2001 may include a UV transparent material that can be etched in a similar manner as second layer 2003, such as fused silica, tantalum pentoxide, or other transparent material. Mask 2002 may include any suitable material that is optically opaque to wavelengths (such as UV light) that may be used to expose a photoresist in a manner such as described with reference to FIGS. 20D-2E, e.g., aluminum, copper, chromium, gold, or the like.

As illustrated in FIG. 20C, a first etching operation may be used to extend the first region of the recess 2010 through the mask layer 2002 and into the first layer 2001 such that the lower surface of the first region is located within the first layer 2001, and the lower surface of the second region of the recess is substantially located at the mask layer 2002. In some examples, the first etching operation may include a first process that removes a portion of the second layer 2003, a second process that removes a portion of the mask layer 2002, and a third process that removes a portion of the first layer 2001. In some examples, the first, second, and third processes may include respective dry etch operations. Dry etch operations for layers 2001 and 2003 may include, for example, use of a CF₄ plasma or a mixture of 90% CF₄ and 10% O₂ plasma. Dry etch operations for layer 2002 may include, for example, use of a chlorine-based plasma (e.g., BCl₃ and Cl₂). Alternatively, layer 2002 may be wet etched, for example with potassium hydroxide (KOH), tetramethylammonium hydroxide (TMAH), nitric acid, acetic acid, or phosphoric acid. When removed as a sacrificial layer, when layer 2002 includes aluminum it may be removed in acidic or basic conditions; when layer 2002 includes copper it may be removed using an iodine and iodide solution or FeCl₃; or when layer 2002 includes gold it may be removed using an iodine and iodide solution.

A first pillar of photoresist then may be formed within the recess 2010. For example, in a manner such as illustrated in FIG. 20D, photoresist precursor 2004 may be disposed at least within the recess 2010, and then exposed to light 2011 in a region corresponding to the first region of the recess and a portion of the second region of the recess. Optionally, the photoresist precursor 2004 may be exposed through the first layer 2001 and through an aperture that the first etching operation forms through the mask layer 2002, in an operation that may be referred to as “back-side exposure.” For example, as illustrated in FIG. 20D, the aperture through mask layer 2002 corresponds to the first region of recess 2010. Mask layer 2002 may inhibit any direct exposure to light 2011 of the photoresist precursor 2004 in a first portion 2007 of the second region of the recess, but may permit some exposure to light 2011 of the photoresist precursor 2004 in a second portion 2008 of the second region of the recess. For example, as illustrated in FIG. 20E, the photoresist precursor 2004 in second portion 2008 of the second region of the recess may be exposed via overexposure to light 2011 through the aperture in the mask and thus form developed photoresist 2005, while the photoresist precursor 2004 in first portion 2007 of the second region may not be exposed and thus may substantially remain undeveloped. The exposed photoresist 2005 forms first pillar 2012. First pillar 2012 may have a first region 2006 substantially filling the first region of the recess 2010 and second region 2008 partially extending into the second region of the recess.

As illustrated in FIG. 20F, photoresist precursor 2004 in first portion 2007 may be removed using a suitable remover, such as DMSO with sonication, acetone, or an NMP based stripper. A second etching operation then may be used to extend the first portion 2007 of the second region of the recess through the mask layer 2002 and into the first layer 2001, such that a lower surface of the first portion 2007 of the second region of the recess is located within the first layer 2001. However, the first region 2006 of the first pillar 2012 inhibits etching of the first region of the recess, and the second region 2008 of the first pillar 2012 inhibits etching of the second portion of the second region of the recess so as to form a wall 2009 located within the first layer 2001 and between the first region of the recess 2010 and the first portion 2007 of the second region of the recess. In some examples, the second etching operation includes a first process that removes a portion of the mask layer 2002, and a second process that removes a portion of the first layer 2001. Unexposed portion(s) of the photoresist precursor 2004 may be removed as part of one or more of such processes, or in a separate process.

In the nonlimiting example illustrated in FIG. 20G, pillar 2012 may be removed, which also may remove the remaining piece of mask layer 2002 which was under pillar 2012, to provide structure 2020 including substrate 2001, first and second recesses 2013, 2014 disposed therein, and wall 2009 between the first recess 2013 and second recess 2014. Wall 2009 may have any suitable dimensions. In some examples, the wall 2009 has a thickness of about 20 nm to about 400 nm between the first recess 2013 (corresponding to the first region of the original recess 2010) and the second recess 2014 (corresponding to the first portion 2007 of the second region of the original recess 2010). Illustratively, the wall 2009 may have a thickness of about 40 nm to about 300 nm between the first recess 2013 (corresponding to the first region of the original recess 2010) and the second recess 2014 (corresponding to the first portion 2007 of the second region of the original recess 2010). For example, the wall 2009 may have a thickness of about 100 nm to about 200 nm between the first recess 2013 (corresponding to the first region of the original recess 2010) and the second recess 2014 (corresponding to the first portion 2007 of the second region of the original recess 2010). The thickness of wall 109 may be controlled, at least in part, by controlling the overexposure described with reference to FIGS. 1D and 1E. The recesses 2013 and 2014 may have any suitable dimensions. In some examples, recesses 2013 and 2014 are about the same size as one another. In other examples, recesses 2013 and 2014 are different sizes than one another.

In some examples, structure 2020 of FIG. 20G may be used in operations such as described in operations 1A-11, 2-4, 5A-5B, 6, 7, 8A-8B, 9A-9F, 10A-10E, 11, 12A-12B, 13-18, 19A-19B, 25A-25G, or 26A-26E, or any other suitable operations described or not specifically described herein. Moreover, any structures such as described with reference to FIGS. 20A-20G may be used in any other suitable operations. That is, structure 2020 is just one example of a structure that can be used in any suitable practical application.

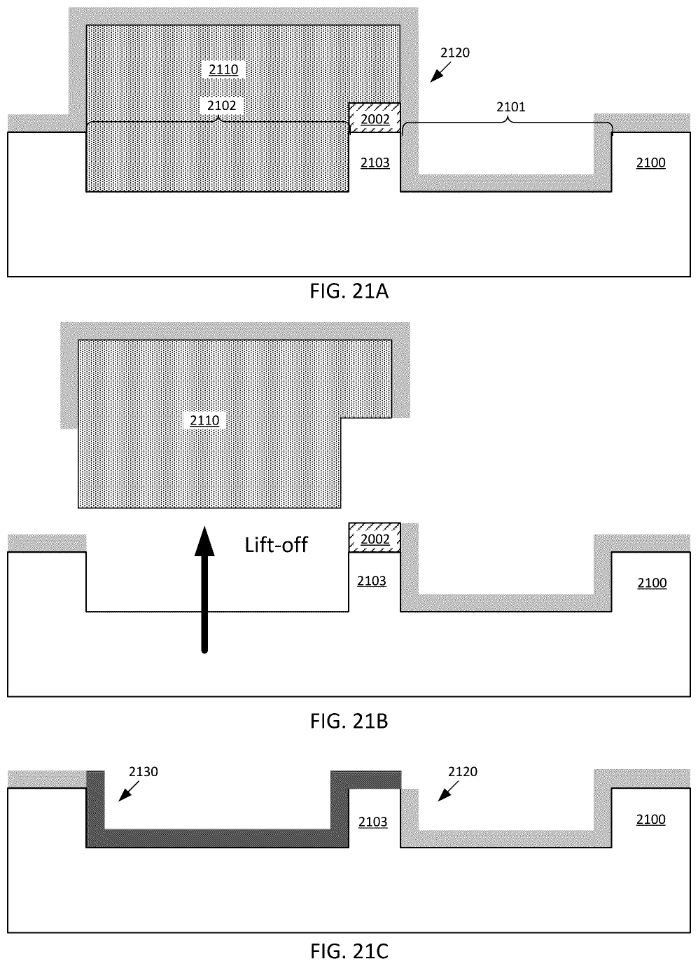

For example, FIGS. 21A-21C schematically illustrate additional example structures and operations for disposing different hydrogels in respective recesses in a substrate. As illustrated in FIG. 21A, beginning with a structure such as described with reference to FIG. 20F (which structure may be formed using operations such as described with reference to FIGS. 20A-20F, or any other suitable combination of operations), a first hydrogel 2120 may be disposed within a first recess 2101 of a substrate 2100 and over a pillar 2110. The substrate 2100 may include a second recess 2102 in which the pillar 2110 is disposed, and a wall 2103 separating the first recess from the second recess. As noted elsewhere herein, the designation “first” or “second” is arbitrary. For example, first recess 2101 illustrated in FIG. 21A may correspond to second recess 2014 of FIG. 20G, and second recess 2102 illustrated in FIG. 21A may correspond to first recess 2013 of FIG. 20G. Substrate 2100, recesses 2101, 2102, wall 2103, and pillar 2110 optionally may be configured in a manner such as described with reference to FIGS. 20A-20G, and optionally may be formed using operations such as described with reference to such figures. As such, FIG. 21A illustrates that a portion of pillar 2102 and a portion of mask layer 2002 are disposed over wall 2103. However, it will be understood that a portion of pillar 2102 need not necessarily be disposed over wall 2103, and that mask layer 2002 may not necessarily be present in all examples, e.g., in examples which are formed using different operations than those described above.

In a manner such as illustrated in FIG. 21B, while the first hydrogel 2120 is disposed within the first recess 2101, pillar 2110 may be removed. Such a process may be referred to as “lift-off” because the pillar may be removed substantially intact from recess 2102. Such lift-off may be performed using a suitable solvent or combination of solvents, such as a mixture of dimethyl sulfoxide (DMSO) and water (e.g., 90% DMSO in water); DMSO with sonication; or an N-methyl-2-pyrrolidone (NMP) based stripper. Any remaining mask layer 2002 optionally may be removed using a suitable acid or base and/or buffer. As illustrated in FIG. 21C, after pillar 2110 is removed and after remaining mask layer 2002 optionally is removed, a second hydrogel 2130 may be disposed within the second recess 2102. Any suitable operations may be used to inhibit second hydrogel 2130 from becoming disposed over first hydrogel 2120. For example, deposition of second hydrogel 2130 may be performed under high ionic strength (e.g., in the presence of 10×PBS, NaCl, KCl, or the like), such that the second hydrogel 2130 substantially does not deposit on or adhere to the first hydrogel 2120. As such, the bare portions illustrated in FIG. 21B selectively receive the second hydrogel 2130. Additionally, or alternatively, the bare portions illustrated in FIG. 21B may be activated to generate surface groups that react with second hydrogel 2130. Wall 2103 may at least partially separate the first hydrogel 2120 from the second hydrogel 2130. In a manner such as described in greater detail with reference to FIG. 18 and as illustrated in FIG. 21C, first hydrogel 2120 may be disposed on sidewalls of recess 2101, the sidewalls being formed at least by the substrate 2100 and wall 2103. Similarly, second hydrogel 2130 may be disposed on sidewalls within recess 2102, the sidewalls being formed at least by substrate and the wall 2103.

The hydrogel 2120 disposed as described with reference to FIG. 21A and the hydrogel disposed as described with reference to FIG. 21B respectively may include any suitable type of hydrogel (such as described elsewhere herein), and may be deposited in any suitable manner, such as by spin-coating, ink-jet (e.g., global precision dispense (GPD) using a GPD tool), spray, slot-die, dip-coating, dunk-coating, and the like. Optionally, substrate 2100 may be pre-treated before disposing hydrogel 2120, e.g., may be functionalized to include moieties that may react with corresponding moieties in hydrogel 2120 so as to covalently couple hydrogel 2120 to the substrate. Similarly, substrate 2100 may be pre-treated after disposing hydrogel 2120, and before disposing hydrogel 2130 e.g., may be functionalized to include moieties that may react with corresponding moieties in hydrogel 2130 so as to covalently couple hydrogel 2130 to the substrate. Additionally, or alternatively, hydrogel 2120 may be modified after being deposited and before hydrogel 2130 is deposited, so as to inhibit chemical reactions between hydrogel 2120 and hydrogel 2130.

Hydrogels 2120 and 2130 optionally may be used in a manner such as described elsewhere herein. For example, the first hydrogel 2120 may be coupled to a first set of capture primers, and the second hydrogel 2130 may be coupled to a second set of capture primers. Capture primers of the first set may be of a different type than capture primers of the second set. For example, the first set of capture primers may include seeding primers 921 and/or 922, and the second set of capture primers may include amplification primers 154 and/or 155, e.g., a mixture of 154 and 155. Or, for example, the first set of capture primers may include a first set of amplification primers (e.g., one of 154 or 155) and the second set of capture primers may include a second set of amplification primers (e.g., the other of 154 or 155). The capture primers may be coupled to the respective hydrogels at any suitable time. For example, the first hydrogel 2120 may be coupled to the first set of capture primers after removing the pillar 2110 from the first recess 2102 and before disposing the second hydrogel within the first portion of the second region of the recess. Additionally, or alternatively, the second hydrogel 2130 may be coupled to the second set of capture primers after the second hydrogel is disposed within recess 2102. In other examples, one of the first and second hydrogels 2120, 2130 is coupled to capture primers (e.g., 921, 922, 154, and/or 155) and the other of the first and second hydrogels is used to capture a hydrogel particle in a manner such as described with reference to FIG. 18. In still other examples, neither of the first and second hydrogels 2120, 2130 is coupled to capture primers (e.g., 921, 922, 154, and/or 155) and instead are used to capture respective hydrogel particles in a manner such as described with reference to FIG. 18.

### WORKING EXAMPLE

The following example is intended to be purely illustrative, and not limiting in any way.

### Example 1

DNA bottlebrush structures such as illustrated in FIG. 27A were prepared with full-length seeding adapters 1056 (seeding adapters 2720) having the sequence PX′. The bottlebrush structures were coupled to a double-stranded polynucleotide 151, 951′ from the PhiX library via a full-length adapter 2356 and a full-length primer 2351 illustrated in FIG. 23A (corresponding to primer 2711 illustrated in FIG. 27A). The PhiX library was prepared in a manner such as shown in FIG. 9A, and were hybridized to a primer with an orthogonal sequence to P5, P7, and PX to primer 2711 illustrated in FIG. 27A prior to bringing the bottlebrush to structure 2300. In a manner similar to that described with reference to FIGS. 23A-23B, each of the DNA bottle-brush structures, having the seeding primers and double-stranded polynucleotide coupled thereto, was brought into contact with structure 2300 having a mixture of P5, P7, and a seeding primer selected from Table 2. Seeding and amplification operations, using strand invasion in accordance with FIGS. 23E-23F, were performed to form an amplified cluster. Excision moieties were used to remove amplicons of one orientation, while leaving amplicons of the other orientation remained coupled to the surface. Sequencing-by-synthesis of the remaining amplicons in the cluster then was initiated, and intensity of fluorescence (also referred to as the sequencing intensity) was measured and used to characterize the number of amplicons in the cluster.

TABLE 2

| Name | Sequence | Seq ID No. |
|---|---|---|
| PX Full | /5Hexynyl/TTTTTT AGG AGG AGG AGG AGG AGG AGG | (SEQ ID |
| (24 bp) | AGG | NO: 9) |
| PX (18 bp) | /5Hexynyl/TTTTTT AGG AGG AGG AGG AGG AGG | (SEQ ID |
| | | NO: 10) |
| PX (11 bp) | /5Hexynyl/TTTTTT AGG AGG AGG AG | (SEQ ID |
| | | NO: 11) |
| PX (9 bp) | /5Hexynyl/TTTTTT AGG AGG AGG | (SEQ ID |
| | | NO: 12) |

For comparison, double-stranded polynucleotides 151, 951′ from the PhiX library were prepared having the same full-length seeding adapters 1056. In a manner similar to that illustrated in FIG. 22A, each of the double-stranded polynucleotides, having the seeding adapters coupled thereto, was brought into contact with structure 2200 having a mixture of P5, P7, and a seeding primer selected from Table 2. Seeding and amplification operations, using strand invasion such as described with reference to FIGS. 9C-9F, were performed to form an amplified cluster. Excision moieties were used to remove amplicons of one orientation, while leaving amplicons of the other orientation remained coupled to the surface. Sequencing-by-synthesis of the remaining amplicons in the cluster then was initiated, and intensity of fluorescence (also referred to as the sequencing intensity) was measured and used to characterize the number of amplicons in the cluster.

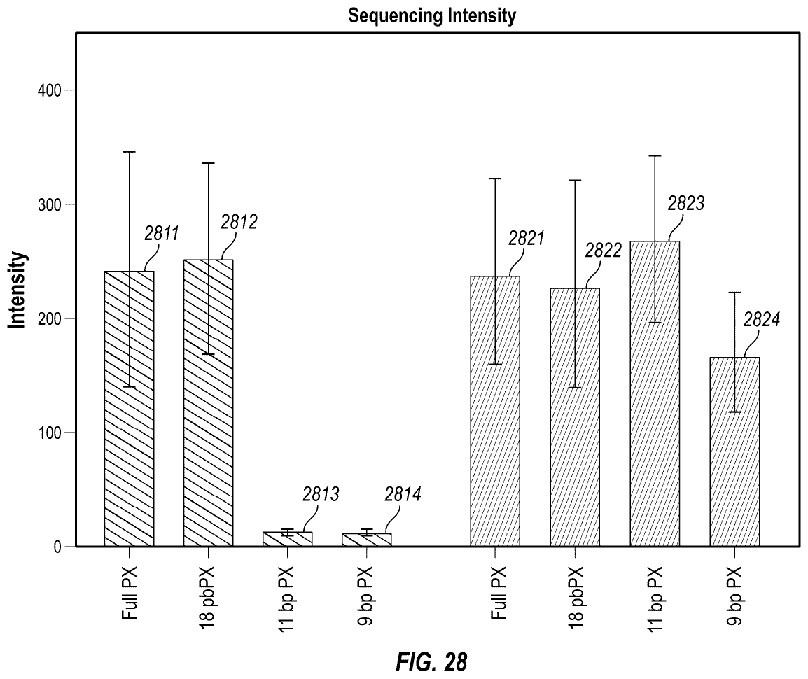

FIG. 28 illustrates sequencing intensity from clusters respectively formed using double-stranded nucleotides or using scaffolds, for different lengths of seeding primers. The error bars in FIG. 28 come from differences across the flowcell lane, hence the averaging across the entire lane for a singular intensity value. Intensity 2811 corresponds to the sequencing intensity for clusters of double-stranded polynucleotides 151, 951′ formed using a surface with the PX Full (24 bp) sequence in Table 2 as seeding primers 921. Intensity 2812 corresponds to the sequencing intensity for clusters of double-stranded polynucleotides 151, 951′ formed using a surface with the PX (18 bp) sequence in Table 2 as seeding primers 921. Intensity 2813 corresponds to the sequencing intensity for clusters of double-stranded polynucleotides 151, 951′ formed using a surface with the PX (11 bp) sequence in Table 2 as seeding primers 921. Intensity 2814 corresponds to the sequencing intensity for clusters of double-stranded polynucleotides 151, 951′ formed using a surface with the PX (9 bp) sequence in Table 2 as seeding primers 921. It can be seen from FIG. 28 that intensities 2811 and 2812 are similar to one another, from which it may be understood that the full-length and 18 bp sequences for primers 921 yielded similar numbers of amplicons in their respective clusters. In comparison, intensities 2813 and 2814 are significantly lower than intensities 2811 and 2812, from which it may be understood that the 11 bp and 9 pb sequences for primers 921 yielded significantly fewer amplicons in their respective clusters than did the full-length and 18 bp sequences.

Intensity 2821 in FIG. 28 corresponds to the sequencing intensity for clusters of double-stranded polynucleotides 151, 951′ formed using the above-described bottlebrush structure and using a surface with the PX Full (24 bp) sequence in Table 2 as seeding primers 921. Intensity 2822 corresponds to the sequencing intensity for clusters of double-stranded polynucleotides 151, 951′ formed using the above-described bottlebrush structure and using a surface with the PX (18 bp) sequence in Table 2 as seeding primers 921. Intensity 2823 corresponds to the sequencing intensity for clusters of double-stranded polynucleotides 151, 951′ formed using a surface with the PX (11 bp) sequence in Table 2 as seeding primers 921. Intensity 2824 corresponds to the sequencing intensity for clusters of double-stranded polynucleotides 151, 951′ formed using the above-described bottlebrush structure and using a surface with the PX (9 bp) sequence in Table 2 as seeding primers 921. It can be seen from FIG. 28 that intensities 2821, 2822, and 2823 are similar to one another, from which it may be understood that the full-length, 18 bp, and 11 bp sequences for primers 921 yielded similar numbers of amplicons in their respective clusters. Although intensity 2824 is somewhat lower than intensities 2821, 2822, and 2823 in this set of examples, the error bars for all four intensities fall roughly in the same range as one another. Additionally, intensities 2821, 2822, 2823, and 2824 are roughly similar to intensities 2811 and 2812, from which it may be understood that using the bottlebrush structure did not detrimentally affect cluster formation. Indeed, intensities 2823 and 2824 (using the bottlebrush structure) are significantly higher than intensities 2813 and 2814 (which did not use the bottlebrush structure), from which it may be understood that the bottlebrush structure was satisfactorily used for sequencing even when seeding was performed using the 11 bp and 9 pb sequences as primers 921. Without wishing to be bound by any theory, it is believed that the bottlebrush structure forms multiple duplexes with primers 921 which sufficiently bind the structure to the surface that the double-stranded polynucleotide coupled thereto may be seeded and then amplified at the surface.

### ADDITIONAL COMMENTS

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of amplifying a polynucleotide, the method comprising:
contacting a structure with a fluid comprising polynucleotides having a variety of lengths, each of the polynucleotides comprising first and second adapters,
wherein the structure comprises:
a substrate comprising a first region and a second region spaced apart from one another by a gap of at least 100 nm;
a first set of capture primers coupled to the first region of the substrate; and
a second set of capture primers coupled to the second region of the substrate;
hybridizing the first adapter of a polynucleotide from the fluid to a capture primer of the first set of capture primers;
based upon that polynucleotide being sufficiently long to bridge the gap, amplifying that polynucleotide using the first set of capture primers and the second set of capture primers; and
based upon that polynucleotide being insufficiently long to bridge the gap, not amplifying that polynucleotide.

2. The method of claim 1, wherein the gap has length A, and based upon the polynucleotide being about A/0.34 nm bases long or greater, the polynucleotide is amplified, and based upon the polynucleotide being less than about A/0.34 nm bases long, the polynucleotide is not amplified.

3. The method of claim 1, wherein the structure further comprises a wall disposed within the gap between the first region and the second region.

4. The method of claim 3, wherein the first region is located within a first recess of the substrate, wherein the second region is located within a second recess of the substrate, and wherein the wall divides the first recess from the second recess.

5. The method of claim 1, wherein the structure further comprises a vertical sidewall comprising the first region or a raised surface comprising the first region.

6. (canceled)

7. The method of claim 1, wherein the first region surrounds the second region or wherein the second region surrounds the first region.

8. (canceled)

9. The method of claim 1, wherein the first region and the second region are at least partially coplanar with one another.

10. The method of claim 1, wherein the first region and the second region are at least partially vertically separated from one another.

11. The method of claim 1, wherein the second set of capture primers comprises a mixture of first and second types of capture primers.

12. The method of claim 11, wherein the first set of capture primers consists essentially of a third type of capture primer.

13. (canceled)

14. The method of claim 1, wherein the first set of capture primers comprises amplification primers of a first type.

15. The method of claim 14, wherein the second set of capture primers comprises amplification primers of a second type that is different from the first type.

16. The method of claim 1, wherein the first set of capture primers comprises seeding primers of a first type.

17. The method of claim 16, wherein the second set of capture primers comprises seeding primers of a second type that is different from the first type of the first seeding primers.

18. The method of claim 17, wherein the first adapter comprises a first seeding adapter which is shorter than the seeding primers of the first type, and the first polynucleotide comprises a second seeding adapter which is shorter than the seeding primers of the second type.

19. (canceled)

20. The method of claim 18, wherein, based upon that polynucleotide being sufficiently long to bridge the gap:
the first seeding adapter and one of the seeding primers of the first type of the seeding primers form a first duplex, and the second seeding adapter and one of the seeding primers of the second type of the seeding primers form a second duplex,
wherein the first and second duplex together sufficiently hold the polynucleotide to the structure to amplify the polynucleotide.

21. The method of claim 18, wherein, based upon that polynucleotide being insufficiently long to bridge the gap:
the first seeding adapter and one of the seeding primers of the first type of the seeding primers form a first duplex, or
the second seeding adapter and one of the seeding primers of the second type of the seeding primers form a second duplex,
wherein the first duplex alone insufficiently holds the polynucleotide to the structure to amplify the polynucleotide, and
wherein the second duplex alone insufficiently holds the polynucleotide to the structure to amplify the polynucleotide.

22. The method of claim 20, wherein:
the first duplex has a melting temperature of about 35-50° C.; and
the second duplex has a melting temperature of about 35-55° C.

23. The method of claim 16, wherein the structure further comprises amplification primers.

24. The method of claim 1, wherein the first adapter is single-stranded.

25. The method of claim 1, wherein the polynucleotide further comprises a second adapter, which is double-stranded.

26. The method of claim 25, wherein a capture primer of the second set of capture primers binds to the second adapter using strand invasion.

27. A structure, comprising:
a substrate comprising a first region and a second region spaced apart from one another by a gap of at least 100 nm;
a first set of capture primers coupled to the first region of the substrate;
a second set of capture primers coupled to the second region of the substrate; and
a polynucleotide comprising a first adapter hybridized to a capture primer of the first set of capture primers, and a second adapter hybridized to a capture primer of the second set of capture primers.

28-138. (canceled)


SEQUENCE LISTING (14 sequences)

SEQ ID NO: 1 - DNA, 20 bp, synthetic construct
  source 1..20: mol_type = other DNA; note = P5 amplification primers
aatgatacggcgaccaccga

SEQ ID NO: 2 - DNA, 21 bp, synthetic construct
  source 1..21: mol_type = other DNA; note = P7 amplification primers
caagcagaagacggcatacga

SEQ ID NO: 3 - DNA, 20 bp, synthetic construct
  source 1..20: mol_type = other DNA; note = complementary P5 adapters
tcggtggtcgccgtatcatt

SEQ ID NO: 4 - DNA, 21 bp, synthetic construct
  source 1..21: mol_type = other DNA; note = complementary P7 adapters
tcgtatgccgtcttctgcttg

SEQ ID NO: 5 - DNA, 24 bp, synthetic construct
  source 1..24: mol_type = other DNA; note = PX primers
aggaggaggaggaggaggaggagg

SEQ ID NO: 6 - DNA, 30 bp, synthetic construct
  source 1..30: mol_type = other DNA; note = PY primers
gaagaagaagaagaagaagaagaagaagaa

SEQ ID NO: 7 - DNA, 24 bp, synthetic construct
  source 1..24: mol_type = other DNA; note = cPX primers
cctcctcctcctcctcctcctcct

SEQ ID NO: 8 - DNA, 30 bp, synthetic construct
  source 1..30: mol_type = other DNA; note = Seeding adapters
ttcttcttcttcttcttcttcttcttcttc

SEQ ID NO: 9 - DNA, 30 bp, synthetic construct
  source 1..30: mol_type = other DNA; note = PX Full (24 bp)
  modified_base 1: mod_base = OTHER; note = 5Hexynyl
ttttttaggaggaggaggaggaggaggagg

SEQ ID NO: 10 - DNA, 24 bp, synthetic construct
  source 1..24: mol_type = other DNA; note = PX (18 bp)
  modified_base 1: mod_base = OTHER; note = 5Hexynyl
ttttttaggaggaggaggaggagg

SEQ ID NO: 11 - DNA, 17 bp, synthetic construct
  source 1..17: mol_type = other DNA; note = PX (11 bp)
  modified_base 1: mod_base = OTHER; note = 5Hexynyl
ttttttaggaggaggag

SEQ ID NO: 12 - DNA, 15 bp, synthetic construct
  source 1..15: mol_type = other DNA; note = PX (9 bp)
  modified_base 1: mod_base = OTHER; note = 5Hexynyl
ttttttaggaggagg

SEQ ID NO: 13 - protein, 13 aa, synthetic construct
  source 1..13: note = SpyTag
AHIVMVDAYKPTK

SEQ ID NO: 14 - protein, 134 aa, synthetic construct
  source 1..134: note = SpyCatcher
MKGSSHHHHHHVDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATK